SEQUENCE LISTING (35 sequences)

SEQ ID NO: 1 - protein, 4 aa, Synthetic construct
GSSG

SEQ ID NO: 2 - protein, 8 aa, Synthetic construct
GSSGSSGS

SEQ ID NO: 3 - protein, 49 aa, Bos taurus
TNKKECPEDYTYNPRCPQQYGWSDCDCMGDRFGGYCRQDGCSNYIHRST

SEQ ID NO: 4 - protein, 211 aa, Synthetic construct
AFFMTSTTNDTDKVDYEEYSTELIVNTDSESTIDIILSGSTHSPETSSKKPDYIDNSNCSSVFEIATPEPITDNVEDHTDTVTYTSDSINTVSASSGESTTDETPEPITDKEDHTVTDTVSYTTVSTSSGIVTTKSTTDDADLYDTYNDNDTVPPTTVGGSTTSISNYKTKDFVEIFGITALIILSAVAIFCITYYIYNKRSRKYKTENKV

SEQ ID NO: 5 - protein, 175 aa, Synthetic construct
METDTLLLWVLLLWVPGSTGDQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSSGSGSGSEHHHHHHHHSGYPYDVPDYA

SEQ ID NO: 6 - protein, 219 aa, Synthetic construct
METDTLLLWVLLLWVPGSTGDQVQLQESGGGLVQAGGSLRLSCAASGRTFSEYAMGWFRQTNKKECPEDYTYNPRCPQQYGWSDCDCMGDRFGGYCRQDGCSNYIHRSTREFVATISWSGGSTYYTDSVKGRFTISRDNAKNTVYLQMNSLKPDDTAVYYCAAAGLGTVVSEWDYDYDYWGQGTQVTVSSGSSGSGSGSEHHHHHHHHSGYPYDVPDYA

SEQ ID NO: 7 - protein, 175 aa, Synthetic construct
METDTLLLWVLLLWVPGSTGDQVQLQESGGGLVQAGGSLRLSCAASGRTFSEYAMGWFRQGSSGEREFVATISWSGGSTYYTDSVKGRFTISRDNAKNTVYLQMNSLKPDDTAVYYCAAAGLGTVVSEWDYDYDYWGQGTQVTVSSGSSGSGSGSEHHHHHHHHSGYPYDVPDYA

SEQ ID NO: 8 - protein, 179 aa, Synthetic construct
METDTLLLWVLLLWVPGSTGDQVQLQESGGGLVQAGGSLRLSCAASGRTFSEYAMGWFRQGSSGSSGSEREFVATISWSGGSTYYTDSVKGRFTISRDNAKNTVYLQMNSLKPDDTAVYYCAAAGLGTVVSEWDYDYDYWGQGTQVTVSSGSSGSGSGSEHHHHHHHHSGYPYDVPDYA

SEQ ID NO: 9 - protein, 398 aa, Synthetic construct
METDTLLLWVLLLWVPGSTGDQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSSGSGSGSEHHHHHHHHSGYPYDVPDYAGSSSGGSSGEGSAFFMTSTTNDTDKVDYEEYSTELIVNTDSESTIDIILSGSTHSPETSSKKPDYIDNSNCSSVFEIATPEPITDNVEDHTDTVTYTSDSINTVSASSGESTTDETPEPITDKEDHTVTDTVSYTTVSTSSGIVTTKSTTDDADLYDTYNDNDTVPPTTVGGSTTSISNYKTKDFVEIFGITALIILSAVAIFCITYYIYNKRSRKYKTENKV

SEQ ID NO: 10 - protein, 442 aa, Synthetic construct
METDTLLLWVLLLWVPGSTGDQVQLQESGGGLVQAGGSLRLSCAASGRTFSEYAMGWFRQTNKKECPEDYTYNPRCPQQYGWSDCDCMGDRFGGYCRQDGCSNYIHRSTREFVATISWSGGSTYYTDSVKGRFTISRDNAKNTVYLQMNSLKPDDTAVYYCAAAGLGTVVSEWDYDYDYWGQGTQVTVSSGSSGSGSGSEHHHHHHHHSGYPYDVPDYAGSSSGGSSGEGSAFFMTSTTNDTDKVDYEEYSTELIVNTDSESTIDIILSGSTHSPETSSKKPDYIDNSNCSSVFEIATPEPITDNVEDHTDTVTYTSDSINTVSASSGESTTDETPEPITDKEDHTVTDTVSYTTVSTSSGIVTTKSTTDDADLYDTYNDNDTVPPTTVGGSTTSISNYKTKDFVEIFGITALIILSAVAIFCITYYIYNKRSRKYKTENKV

SEQ ID NO: 11 - protein, 398 aa, Synthetic construct
METDTLLLWVLLLWVPGSTGDQVQLQESGGGLVQAGGSLRLSCAASGRTFSEYAMGWFRQGSSGEREFVATISWSGGSTYYTDSVKGRFTISRDNAKNTVYLQMNSLKPDDTAVYYCAAAGLGTVVSEWDYDYDYWGQGTQVTVSSGSSGSGSGSEHHHHHHHHSGYPYDVPDYAGSSSGGSSGEGSAFFMTSTTNDTDKVDYEEYSTELIVNTDSESTIDIILSGSTHSPETSSKKPDYIDNSNCSSVFEIATPEPITDNVEDHTDTVTYTSDSINTVSASSGESTTDETPEPITDKEDHTVTDTVSYTTVSTSSGIVTTKSTTDDADLYDTYNDNDTVPPTTVGGSTTSISNYKTKDFVEIFGITALIILSAVAIFCITYYIYNKRSRKYKTENKV

SEQ ID NO: 12 - protein, 402 aa, Synthetic construct
METDTLLLWVLLLWVPGSTGDQVQLQESGGGLVQAGGSLRLSCAASGRTFSEYAMGWFRQGSSGSSGSEREFVATISWSGGSTYYTDSVKGRFTISRDNAKNTVYLQMNSLKPDDTAVYYCAAAGLGTVVSEWDYDYDYWGQGTQVTVSSGSSGSGSGSEHHHHHHHHSGYPYDVPDYAGSSSGGSSGEGSAFFMTSTTNDTDKVDYEEYSTELIVNTDSESTIDIILSGSTHSPETSSKKPDYIDNSNCSSVFEIATPEPITDNVEDHTDTVTYTSDSINTVSASSGESTTDETPEPITDKEDHTVTDTVSYTTVSTSSGIVTTKSTTDDADLYDTYNDNDTVPPTTVGGSTTSISNYKTKDFVEIFGITALIILSAVAIFCITYYIYNKRSRKYKTENKV

SEQ ID NO: 13 - protein, 218 aa, Synthetic construct
METDTLLLWVLLLWVPGSTGDQVQLQESGGGLVQAGGSLRLSCAASGRTFSEYAMGWFRTNKKECPEDYTYNPRCPQQYGWSDCDCMGDRFGGYCRQDGCSNYIHRSTREFVATISWSGGSTYYTDSVKGRFTISRDNAKNTVYLQMNSLKPDDTAVYYCAAAGLGTVVSEWDYDYDYWGQGTQVTVSSGSSGSGSGSEHHHHHHHHSGYPYDVPDYA

SEQ ID NO: 14 - protein, 187 aa, Synthetic construct
METDTLLLWVLLLWVPGSTGDQVQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYDYWGQGTQVTVSSGSSGSGSGSESAWSHPQFEKGGGSGGGSGGSSAWSHPQFEK

SEQ ID NO: 15 - protein, 231 aa, Synthetic construct
METDTLLLWVLLLWVPGSTGDQVQLQESGGGLVQAGGSLRLSCAASGRTFSEYAMGWFRQTNKKECPEDYTYNPRCPQQYGWSDCDCMGDRFGGYCRQDGCSNYIHRSTREFVATISWSGGSTYYTDSVKGRFTISRDNAKNTVYLQMNSLKPDDTAVYYCAAAGLGTVVSEWDYDYDYWGQGTQVTVSSGSSGSGSGSESAWSHPQFEKGGGSGGGSGGSSAWSHPQFEK

SEQ ID NO: 16 - protein, 435 aa, Synthetic construct
METDTLLLWVLLLWVPGSTGEVQLQESGGGLVQAGGSLRLSCAASGRTFSEYAMGWFRQTNKKECPEDYTYNPRCPQQYGWSDCDCMGDRFGGYCRQDGCSNYIHRSTREFVATISWSGGSTYYTDSVKGRFTISRDNAKNTVYLQMNSLKPDDTAVYYCAAAGLGTVVSEWDYDYDYWGQGTQVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 420 aa, Synthetic construct
METDTLLLWVLLLWVPGSTGEVQLQESGGGLVQAGGSLRLSCAASGRTFSEYAMGWFRQTNKKECPEDYTYNPRCPQQYGWSDCDCMGDRFGGYCRQDGCSNYIHRSTREFVATISWSGGSTYYTDSVKGRFTISRDNAKNTVYLQMNSLKPDDTAVYYCAAAGLGTVVSEWDYDYDYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 48 aa, Bos taurus
NKLKSCPDGYTSGVECRFRGYTCANDGCWRVCSFTTCSGWMPASDTYR

SEQ ID NO: 19 - protein, 181 aa, Synthetic construct
METDTLLLWVLLLWVPGSTGQVQLQESGGGSVQAGGSLRLSCVASGSIFSINAMDWYRQAPGKQRELVAGITSGGSTNYGDFVKGRFTISRDNAKNTVYLQMDSLKPEDTAVYYCAAEVGGWGPPRPDYWGHGTQVTVSSGSSGSGSGSESAWSHPQFEKGGGSGGGSGGSSAWSHPQFEK

SEQ ID NO: 20 - protein, 225 aa, Synthetic construct
METDTLLLWVLLLWVPGSTGQVQLQESGGGSVQAGGSLRLSCVASGSIFSINAMDWYRQTNKKECPEDYTYNPRCPQQYGWSDCDCMGDRFGGYCRQDGCSNYIHRSTRELVAGITSGGSTNYGDFVKGRFTISRDNAKNTVYLQMDSLKPEDTAVYYCAAEVGGWGPPRPDYWGHGTQVTVSSGSSGSGSGSESAWSHPQFEKGGGSGGGSGGSSAWSHPQFEK

SEQ ID NO: 21 - protein, 225 aa, Synthetic construct
METDTLLLWVLLLWVPGSTGQVQLQESGGGSVQAGGSLRLSCVASGSIFSINAMDWYRQTNKLKSCPDGYTSGVECRFRGYTCANDGCWRVCSFTTCSGWMPASDTYRRELVAGITSGGSTNYGDFVKGRFTISRDNAKNTVYLQMDSLKPEDTAVYYCAAEVGGWGPPRPDYWGHGTQVTVSSGSSGSGSGSESAWSHPQFEKGGGSGGGSGGSSAWSHPQFEK

SEQ ID NO: 22 - protein, 230 aa, Synthetic construct
METDTLLLWVLLLWVPGSTGDQVQLQESGGGLVQAGGSLRLSCAASGRTFSEYAMGWFRQNKLKSCPDGYTSGVECRFRGYTCANDGCWRVCSFTTCSGWMPASDTYRREFVATISWSGGSTYYTDSVKGRFTISRDNAKNTVYLQMNSLKPDDTAVYYCAAAGLGTVVSEWDYDYDYWGQGTQVTVSSGSSGSGSGSESAWSHPQFEKGGGSGGGSGGSSAWSHPQFEK

SEQ ID NO: 23 - protein, 177 aa, Synthetic construct
METDTLLLWVLLLWVPGSTGAWQLVESGGGSVQPGGSLTLTCQASKSTFSTSGMRWERQAQGKGVEFVADISSDSTRKWYSDSVKGRFTISRSNWWRTVTLQMNDLKPEDTARYYCKDLESHHLRGQGTQVTVSSSGSSGSGSGSESAWSHPQFEKGGGSGGGSGGSSAWSHPQFEK

SEQ ID NO: 24 - protein, 221 aa, Synthetic construct
METDTLLLWVLLLWVPGSTGAWQLVESGGGSVQPGGSLTLTCQASKSTFSTSGMRWERQTNKKECPEDYTYNPRCPQQYGWSDCDCMGDRFGGYCRQDGCSNYIHRSTVEFVADISSDSTRKWYSDSVKGRFTISRSNWWRTVTLQMNDLKPEDTARYYCKDLESHHLRGQGTQVTVSSSGSSGSGSGSESAWSHPQFEKGGGSGGGSGGSSAWSHPQFEK

SEQ ID NO: 25 - protein, 235 aa, Synthetic construct
MGDNDIHFAFLSTGAHSSYELTQPSSVSGSLGQRVSVTCSGSSSNVGNGYVSWYQLIPGSAPRTIIYGDTSRASGVPERFSGSRSGNTATLTISSLQAEDEADFFCASPDDSSSNAVFGSGTTLTVLRTGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 515 aa, Synthetic construct
MGDNDIHFAFLSTGAHSQVQLRESGPSLMKPSQTLSLTCTVSGSSLNDKSVGWVRQAPGKALQWLGSVDTSGNTDYNPGLKSRLSITKDNSKSRISLTVTGMTTEDSATYYCITAHQKTNKKECPEDYTYNPRCPQQYGWSDCDCMGDRFGGYCRQDGCSNYIHRSTYEWYVSAWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 230 aa, Synthetic construct
MGDNDIHFAFLSTGAHSDIQMTQSPSTLSASVGDRVTITCRASQSISSWLAWYQQKPGEAPKLLISDASSLESGVPSRFSGSGSGTEFTLTISSLQPDDFATYYCQQYYSSPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 469 aa, Synthetic construct
MGDNDIHFAFLSTGAHSEVQLVESGGGLVQPGGSLRVSCAASGFTFSSYAMSWVRQAPGKGLEWVSAISGLGGSTYYADSVKGRFTISRDNSKNTLYLQMNSLRAEDTAVYYCAKDHRVWAAGYHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 304 aa, Synthetic construct
METDTLLLWVLLLWVPGSTGDIQMTQSPSTLSASVGDRVTITCRASQSISSWLAWYQQKPGEAPKLLISDASSLESGVPSRFSGSGSGTEFTLTISSLQPDDFATYYCQQYYSSPTFGGGTKVEIKGGGGSGGGGSGGGGSEVQLVESGGGLVQPGGSLRVSCAASGFTFSSYAMSWVRQAPGKGLEWVSAISGLGGSTYYADSVKGRFTISRDNSKNTLYLQMNSLRAEDTAVYYCAKDHRVWAAGYHFDYWGQGTLVTVSSGSSGSGSGSESAWSHPQFEKGGGSGGGSGGSSAWSHPQFEK

SEQ ID NO: 30 - protein, 509 aa, Synthetic construct
METDTLLLWVLLLWVPGSTGDIQMTQSPSTLSASVGDRVTITCRASQSISSWLAWYQQKPGEAPKLLISDASSLESGVPSRFSGSGSGTEFTLTISSLQPDDFATYYCQQYYSSPTFGGGTKVEIKGGGGSGGGGSGGGGSEVQLVESGGGLVQPGGSLRVSCAASGFTFSSYAMSWVRQAPGKGLEWVSAISGLGGSTYYADSVKGRFTISRDNSKNTLYLQMNSLRAEDTAVYYCAKDHRVWAAGYHFDYWGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 348 aa, Synthetic construct
METDTLLLWVLLLWVPGSTGDIQMTQSPSTLSASVGDRVTITCRASQSISSWLAWYQQKPGEAPKLLISDASSLESGVPSRFSGSGSGTEFTLTISSLQPDDFATYYCQQYYSSPTFGGGTKVEIKGGGGSGGGGSGGGGSEVQLVESGGGLVQPGGSLRVSCAASGFTFSSYAMSWVRQTNKKECPEDYTYNPRCPQQYGWSDCDCMGDRFGGYCRQDGCSNYIHRSTLEWVSAISGLGGSTYYADSVKGRFTISRDNSKNTLYLQMNSLRAEDTAVYYCAKDHRVWAAGYHFDYWGQGTLVTVSSGSSGSGSGSESAWSHPQFEKGGGSGGGSGGSSAWSHPQFEK

SEQ ID NO: 32 - protein, 348 aa, Synthetic construct
METDTLLLWVLLLWVPGSTGDIQMTQSPSTLSASVGDRVTITCRASQSISSWLAWYQQTNKKECPEDYTYNPRCPQQYGWSDCDCMGDRFGGYCRQDGCSNYIHRSTVKLLISDASSLESGVPSRFSGSGSGTEFTLTISSLQPDDFATYYCQQYYSSPTFGGGTKVEIKGGGGSGGGGSGGGGSEVQLVESGGGLVQPGGSLRVSCAASGFTFSSYAMSWVRQAPGKGLEWVSAISGLGGSTYYADSVKGRFTISRDNSKNTLYLQMNSLRAEDTAVYYCAKDHRVWAAGYHFDYWGQGTLVTVSSGSSGSGSGSESAWSHPQFEKGGGSGGGSGGSSAWSHPQFEK

SEQ ID NO: 33 - protein, 553 aa, Synthetic construct
METDTLLLWVLLLWVPGSTGDIQMTQSPSTLSASVGDRVTITCRASQSISSWLAWYQQKPGEAPKLLISDASSLESGVPSRFSGSGSGTEFTLTISSLQPDDFATYYCQQYYSSPTFGGGTKVEIKGGGGSGGGGSGGGGSEVQLVESGGGLVQPGGSLRVSCAASGFTFSSYAMSWVRQTNKKECPEDYTYNPRCPQQYGWSDCDCMGDRFGGYCRQDGCSNYIHRSTLEWVSAISGLGGSTYYADSVKGRFTISRDNSKNTLYLQMNSLRAEDTAVYYCAKDHRVWAAGYHFDYWGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 553 aa, Synthetic construct
METDTLLLWVLLLWVPGSTGDIQMTQSPSTLSASVGDRVTITCRASQSISSWLAWYQQTNKKECPEDYTYNPRCPQQYGWSDCDCMGDRFGGYCRQDGCSNYIHRSTVKLLISDASSLESGVPSRFSGSGSGTEFTLTISSLQPDDFATYYCQQYYSSPTFGGGTKVEIKGGGGSGGGGSGGGGSEVQLVESGGGLVQPGGSLRVSCAASGFTFSSYAMSWVRQAPGKGLEWVSAISGLGGSTYYADSVKGRFTISRDNSKNTLYLQMNSLRAEDTAVYYCAKDHRVWAAGYHFDYWGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 194 aa, Synthetic construct
METDTLLLWVLLLWVPGSTGDQVQLQESGGGLVQAGGSLRLSCAASGRTFSEYAMGWFRQGSSGNSTGSSGEREFVATISWSGGSTYYTDSVKGRFTISRDNAKNTVYLQMNSLKPDDTAVYYCAAAGLGTVVSEWDYDYDYWGQGTQVTVSSGSSGSGSGSESAWSHPQFEKGGGSGGGSGGSSAWSHPQFEK

(19) United States
(12) Patent Application Publication
(10) Pub. No.: US 2025/0228955 A1
(43) Pub. Date: Jul. 17, 2025

(54) PLATFORM TECHNOLOGY FOR BISPECIFIC ANTIGENBINDING PROTEINS

(71) Applicants: ALBERT EINSTEIN COLLEGE OF MEDICINE, Bronx, NY (US); EITR BIOLOGICS, INC., San Diego, CA (US)

(72) Inventors: Kartik Chandran, Brooklyn, NY (US); Zachary Bornholdt, Encinitas, CA (US); Crystal Moyer, San Diego, CA (US); Megan Demouth, New York, NY (US)

(73) Assignees: ALBERT EINSTEIN COLLEGE OF MEDICINE, Bronx, NY (US); EITR BIOLOGICS, INC., San Diego, CA (US)

(21) Appl. No.: 18/980,683

(22) Filed: Dec. 13, 2024

Related U.S. Application Data
(63) Continuation of application No. PCT/US2023/068819, filed on Jun. 21, 2023.
Application No. PCT/US2023/068819 is a continuation of application No. 63/353,989, filed on Jun. 21, 2022.

(51) Int. Cl. A61K 47/68; C07K 14/705; C07K 14/725; C07K 16/10

(52) U.S. Cl. CPC A61K 47/6801; A61K 47/6841; C07K 14/7051; C07K 14/70521; C07K 14/70532; C07K 16/1003; C07K 16/1045; C07K 2317/41; C07K 2317/569; C07K 2318/00; C07K 2319/22; C07K 2319/41; C07K 2319/42; C07K 2319/43; C07K 2319/70

(57) ABSTRACT

Provided herein is a novel platform for engineered antigen-binding proteins (e.g., bispecific antigen-binding proteins, e.g., single-domain bispecifics) called ODIN (Orthogonal Dual-Interacting Nanotherapeutics). The platform is useful in generating a wide range of engineered antigen-binding proteins.

### CROSS-REFERENCE TO RELATED APPLICATIONS

This application claims priority to, and is a 35 U.S.C. § 111(a) continuation of, PCT international application number PCT/US2023/068819 filed on Jun. 21, 2023, incorporated herein by reference in its entirety, which claims priority to, and the benefit of, U.S. provisional patent application Ser. No. 63/353,989 filed on Jun. 21, 2022, incorporated herein by reference in its entirety. Priority is claimed to each of the foregoing applications.

The above-referenced PCT international application was published as PCT International Publication No. WO 2023/250380 A2 on Dec. 28, 2023, which publication is incorporated herein by reference in its entirety.

### STATEMENT REGARDING FEDERALLY SPONSORED RESEARCH OR DEVELOPMENT

This invention was made with Government support under grant number AI132256, awarded by The National Institutes of Health. The Government has certain rights in the invention.

### INCORPORATION-BY-REFERENCE OF SEQUENCE LISTING

This application includes a sequence listing in a file entitled “EIT6627-01PCT-seq-listing.xml” created on Jun. 8, 2023 and having a 39 KB file size. The sequence listing is submitted electronically through Patent Center and is incorporated herein by reference in its entirety.

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to compositions, systems and methods for targeted therapies and research tools and more particularly to bispecific scaffold platform compositions that are stable, biologically active and contain regions that are structurally amenable to insertion of various heterologous sequences, including the sequences of different antigen-binding proteins.

### 2. Background

Bispecific molecules, such as engineered antibodies, are designed to recognize and engage two different antigens or two distinct epitopes on the same antigen with one molecule. For example, bispecific antibodies have been created to enrich delivery of chemotherapeutic or radiotherapeutic agents or toxins to targeted tissues.

Conventional bispecific antibodies are typically produced with the co-expression of heavy chain-light chain pairs with different specificities and assembled. However, a major difficulty with this approach is the inability of producing and purifying materials in sufficient quantities and suitable quality for therapeutic or diagnostic uses.

Current bispecific platforms are based either on large multi-subunit assemblages (e.g., IgGs) or smaller single-domain molecules (e.g., single-chain Fvs, affibodies, monobodies) connected by flexible linkers. The large size and complexity of these molecules can also be significant liabilities for their biological activity. For example, these molecules suffer from reduced biodistribution and tissue penetration. Moreover, the biophysical instabilities and increased aggregation of these molecules pose challenges to their manufacturability as well as increase the cost of goods due to the added steps of purification or the need to manufacture them in mammalian cells. They are also problematic for field deployment due to the need for cold chains to maintain shelf life.

Accordingly, a great need exists in the art for bispecific platforms that provide stable and active biomolecules that maintain a small molecular weight for diagnostic and therapeutic applications.

### BRIEF SUMMARY

The present disclosure is based, at least in part, on the discovery that a certain region in the VH, VL, VNAR, or VHH framework is structurally amenable to insertion of various heterologous sequences, including the sequences of an antigen-binding protein. For example, it is presented herein that said VH, VL, VNAR, or VHH framework allows the insertion of a heterologous antigen-binding protein, such as ‘stalk-and-knob’ sequences derived from the ultralong CDR3 sequences found in a subset of bovine IgG antibodies (‘picobodies’), without disrupting the structure of VH, VL, VNAR, or VHH, or its capacity to recognize its cognate antigen. Importantly, the newly inserted heterologous antigen-binding protein also retains its antigen-binding activity. Accordingly, such a framework provides a novel platform for various engineered proteins including, but are not limited to, bispecific antigen-binding proteins with the potential for enhanced stability and solubility, rapid biodistribution, and low-cost manufacturing.

Using the platform described herein, an exemplary bispecific antigen-binding protein has been created. Specifically, the bispecific antigen-binding protein incorporates orthogonal binding specificities into a single globular ˜20-25 kDa domain, obviating the need to connect multiple domains through flexible linkers. This scaffold is inherently modular, allowing for any antigen-binding domain, e.g., a bovine ultralong CDR3, to be swapped into the engineered position in any VH, VL, VNAR, or VHH framework.

The platform can produce constructs that can be specifically engineered for use as diagnostic or therapeutic materials that may be part of medical treatments for cancer, inflammation disorders, neurological disorders and the like. Constructs may also be part of pharmaceutical compositions, and time release formulations that may be included in kits.

As used herein, the term “conjoint”, with respect to administration of two or more agents, refers to the simultaneous, sequential or separate dosing of the individual agents provided that some overlap occurs in the simultaneous presence of the agents or compositions in a cell or a subject. Accordingly, the term “conjoint therapy”, as used herein, refers to the administration of two or more therapeutic substances. The different agents comprising the conjoint therapy may be administered concomitant with, prior to, or following the administration of one or more therapeutic agents, such that some overlap occurs in the simultaneous presence of the agents in a cell or a subject.

By “detectable label” means a compound, substance, or composition that, when linked to a molecule of interest, renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an E LISA), biotin, digoxigenin, or haptens.

As used herein, the term “Fc region” describes a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. Suitable native-sequence Fc regions for use in the antibodies of the present disclosure include human IgG1, IgG2 (IgG2A, IgG2B), IgG3 and IgG4.

As used herein, the term “FcRnBP” refers to an engineered FcRN-binding peptide, which when fused to a protein, extends the half-life of the protein in plasma. Exemplary peptides are described in the art. In some embodiments, such peptides include those having an amino acid sequence of QRFCTGHFGGLYPCNG; QRFCTGHFGGLHPCNG; QRFVTGHFGGLYPANG; or QRFVTGHFGGLHPANG. The FcRnBP can be linear or cyclical.

As used herein, the term “heterologous polypeptide” refers to a polypeptide that is not natively present at the site of insertion in the target protein. The term “heterologous polypeptide” is not limited by the number of amino acids. Accordingly, the term “heterologous polypeptide” includes, but is not limited to, a single amino acid (e.g., natural or non-natural amino acid), a short peptide comprising 2-50 amino acids (e.g., linker), and a protein comprising 51 amino acids or more (e.g., protein domain/subdomain, or a full-length protein). In certain embodiments, the heterologous polypeptide comprises an antigen-binding protein or a fragment thereof. The heterologous polypeptides of the present disclosure may be inserted into the insertion site with or without a deletion of at least one amino acid residue at the insertion site.

As used herein, the term “K_{D}” is intended to refer to the dissociation equilibrium constant of a particular antibody-antigen interaction. The binding affinity of antibodies of the disclosed technology may be measured or determined by standard antibody-antigen assays, for example, competitive assays, saturation assays, or standard immunoassays such as E LISA or RIA.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

As used herein, the term “polypeptide” is not limited by the number of amino acids. Accordingly, the term “polypeptide” includes a single amino acid (e.g., natural or non-natural amino acid), a short peptide comprising 2-50 amino acids (e.g., linker), and a protein comprising 51 amino acids or more (e.g., protein domain/subdomain, or a full-length protein). The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “selective” refers to a preferential action or function. The term “selective” can be quantified in terms of the preferential effect in a particular target of interest relative to other targets. For example, a measured variable (e.g., binding of an engineered antigen-binding protein of the present disclosure) can be 10%, 20%, 25%, 30%, 35%, 40%, 45%, 50%, 55%, 60%, 65%, 70%, 75%, 80%, 85%, 90%, 95%, 1-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 70-fold, 80-fold, 90-fold, 100-fold, or greater or any range in between inclusive (e.g., 50% to 16-fold), different in a target of interest versus unintended or undesired targets. The same fold analysis can be used to confirm the magnitude of an effect in a given tissue, cell population, measured variable, measured effect, and the like.

By contrast, the term “specific” refers to an exclusionary action or function. For example, specific binding of an antibody or antigen-binding protein to a predetermined antigen refers to the ability of the antibody or antigen-binding protein to bind to the antigen of interest without binding to other antigens. Typically, the antibody binds with an affinity (K_{D}) of approximately less than 1×10⁻⁷ M, such as approximately less than 10⁻⁸ M, 10⁻⁹ M, 10⁻¹⁰ M, 10⁻¹¹ M, or even lower to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen.

As used herein, “subject” refers to any healthy animal, mammal or human, or any animal (e.g., livestock, a dog, a cat), mammal or human afflicted with a cancer. The term “subject” is interchangeable with “patient.”

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically-effective amount” and “effective amount” as used herein means that amount of a compound, material, or composition comprising a compound encompassed by the present disclosure which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. Cancer cell death can be promoted by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in cancer cell numbers and/or a solid malignancy can be achieved.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1A is a schematic depiction of a conventional human antibody.

FIG. 1B is a schematic depiction of a camelid heavy-chain antibody and derived VHH according to one embodiment of the present technology.

FIG. 1C is a schematic depiction of a bovine antibody with ultralong CDR3 and derived picobody according to one embodiment of the present technology.

FIG. 2A depicts a bispecific VHH-based platform with tandem VHH fusions according to one embodiment of the technology.

FIG. 2B depicts a bispecific VHH-based platform bsFab according to one embodiment of the technology.

FIG. 2C depicts a bispecific VHH-based platform LightT cell exchanger (LITE) according to one embodiment of the technology.

FIG. 2D depicts a bispecific VHH-based platform S-Fab according to one embodiment of the technology.

FIG. 2E depicts a bispecific VHH-based platform Bispecific VHH-Fc fusion according to one embodiment of the technology.

FIG. 2F depicts a bispecific VHH-based platform Bispecific VHH-Fc according to one embodiment of the technology.

FIG. 2G depicts a bispecific VHH-based platform Bispecific VHH-FcFy (BiHc) according to one embodiment of the technology.

FIG. 3A depicts an ODIN single-domain bispecific showing a novel insertion site in the framework that highlighted with dashes (between beta sheets C and D).

FIG. 3B depicts schematically the structural organization of VHHs and shows a tandem VHH fusion. Complementarity-determining regions (CDRs) involved in antigen recognition are highlighted.

FIG. 3C shows a structural model of the ODIN VHH-picobody and chimeric ODIN VHH engineered to display bovine ‘stalk and knob’ picobody.

FIG. 4A is a schematic diagram of a VHH display on recombinant vaccinia virus (rVACV) particles (H8, 8× histidine tag. HA, HA epitope tag and TM, transmembrane domain).

FIG. 4B is a panel of a HA tag immunoblot of concentrated rVACVs displaying VHHs, rVACV (WT), and negative control virus. Top panel, HA tag immunoblot of concentrated rVACVs displaying VHHs. rVACV (WT), negative control virus. Bottom panel, immunoblot of vaccinia envelope protein B5 as loading control.

FIG. 4C shows a plot of the capture of SARS-2 spike proteins by rVACVs coated on ELISA plates. Recognition by recombinant spike proteins.

FIG. 4D shows a plot of the capture of HIV-1 spike proteins by rVACVs coated on ELISA plates.

FIG. 4E shows the capture of rVACVs by HIV-1 spike coated on ELISA plates and the detection of bound rVACVs with SARS-2 spike.

FIG. 5A is a coomassie-stained gel of secreted VHHs purified from mammalian cell supernatants. ODIN-2 and ODIN-3 respectively bear 4- and 8-amino glycine-serine (GSSG and GSSGSSGS, respectively) linkers in the C-C′ loop of the VHH framework that is shown in FIG. 2 illustrating the antigen-binding properties of secreted recombinant VHHs.

FIG. 5B is a plot displaying positive high affinity binding of ODIN-1 to the SARS-CoV-2 by biolayer interferometry.

FIG. 5C is a plot displaying positive high affinity binding of ODIN-1 to the HIV-1 spike by biolayer interferometry illustrating antigen-binding properties of secreted recombinant VHHs.

FIG. 5D The binding kinetics of the ODIN-1 Fc region fusion (ODIN-1 Fc) binding to the HIV-1 spike by biolayer interferometry is shown.

FIG. 5E is a plot of sandwich biolayer interferometry experiments that are shown with HIV-1 BG505 spike. These data confirm a sub nanomolar affinity to the HIV-1 spike.

FIG. 5F is a plot of sandwich biolayer interferometry experiments that are shown with SARS-CoV-2 spike loaded on the tip. Both experiments from FIG. 5E and FIG. 5F demonstrate that ODIN-1 can simultaneously bridge two soluble antigens to create a tripartite complex.

FIG. 6A illustrates the sequence conservation of the representative framework region 2 (FR2) loops in camelid VHH antibodies where 227 unique VHH sequences were extracted from the Protein Data Bank (PDB).

FIG. 6B illustrates the sequence conservation of the representative framework region 2 (FR2) loops in human and VHH antibodies where 1531 unique human VH3-23 sequences were extracted from the circulating B cell population of a single individual (PMID: 30664748). Sequences were aligned with Clustal Omega (PMID: 21988835), and logo plots were generated with Weblogo (PMID: 15173120). Alignments corresponding to VH residues 39-55 (IMGT numbering) are shown.

FIG. 7A and FIG. 7B show the structural conservation of FR2 loop into which antigen-binding sequences, including bovine ultralong CDR3s (UL-CDR3s) can be inserted. The display and alignment of a camelid heavy chain only variable region (VHH) variable heavy (V_{H}) and variable light (V_{L}) region structures as well as shark variable new antigen receptor (VNAR) domains. In FIG. 7C the insertion loop structure is shaded in each and encompassed by a broken circle. The antibody CDRs associated with antigen recognition are shown in the larger broken circle in FIG. 7C. The alignment of all these structures shows the remarkable level of conservation for the structure and location of the insertion loop site. The minimal required structure for the insertion site is the ‘ascending’ beta-sheet to loop to ‘descending’ beta-sheet found in that repeated motif observed across these variable region structures and is not an uncommon motif found across other diverse protein structures. The PDB ID codes for the structures displayed are as follows: 6WAQ (VHH), 7SPO (VNAR), 4P49 (Chicken VH and VL), 1HZH (Human VH and VL), 7KKZ (Mouse VH and VL), 4ZTP (RabbitVL and VH), and 1BFO (Rat VL and VH). All the structures were aligned to the structure of the VHH, (PDB ID: 6WAQ).

FIG. 8A is a depiction of a full 150 kDa IgG structure according to the technology.

FIG. 8B is a schematic illustration of a full camelid heavy chain antibody depicting the derivation of the 15 kDa VHH (“nanobody”).’

FIG. 8C is a schematic illustration of a bovine IgG with an UL-CDR3 depicting the derivation of the ˜6 kDa UL-CDR3 stalk-and-knob structure.

FIG. 8D is a schematic illustration of a prototype ODIN bispecific combines the nanobody and bovine UL-CDR3 into a small bispecific sdAb, retaining the binding specificities from the original nanobody and UL-CDR3 sequences.

FIG. 8E is a schematic illustration of two single chain ODIN bispecific molecules that are fused to an Fc region via their C-termini.

FIG. 8F is a schematic illustration of two additional single chain ODIN bispecific molecules are fused to the C-terminus of the Fc region of the molecule in FIG. 8E, yielding an antigen-binding protein comprising four single chain ODIN bispecific molecules.

FIG. 9A is a schematic illustration of four types of shark VNAR domains. All four types contain a disulfide-bonded pair of canonical cysteine residues in FW1 and FW3b. These are indicated by an open circle. Non-canonical pairs of cysteine residues are shown in matching symbols as follows: filled square [FW2-CDR3] and open square [CDR3-FW4] in Type I, filled triangle [CDR1-CDR3] in Type II, and filled diamond [CDR1-CDR3] in Type III. Type IV VNARs solely contain the canonical cysteine residues. Type III sequences are further characterized by a conserved tryptophan residue in CDR1 (indicated by an *).

FIG. 9B illustrates representative sequences of the different types of VNARs obtained from the PDB (1SQ2, 6X4G, 2COQ, 5L8L, 1VER, 1VES) or Genbank (AAM77191). The boundaries of the framework and CDR regions are denoted by the dashed lines extending down from FIG. 9A. Cysteine residues are underlined. A region comprising the potential insertion sites in the VNAR sequences bridges FW2-HV2 and is indicated by italicized residues and the bracket below the alignment. As used herein, the FW2/HV2 region (as demarcated in FIG. 9A and FIG. 9B) comprises 19 amino acids, labeled as amino acid residues 1-19 (FW2 region comprising the amino acid residues 1-11; and HV2 region comprising the amino acid residues 12-19).

FIG. 10 depicts schematic diagrams of the ODIN constructs utilized to demonstrate the ODIN concept.

FIG. 11A is a plot of a neutralization assay of rVSV pseudotyped with either SARS-CoV-2 or MERS-CoV spike proteins. ODIN-5 was tested for neutralization potency against rVSV SARS-CoV-2 and MERS-CoV and the neutralization curves are graphed for each.

FIG. 11B is a plot of a neutralization assay showing the neutralization potency of VHH-55 (a VHH specific for MERS-CoV, positive control) and ODIN derivatives, ODIN-5 (negative control), ODIN-6, and ODIN-7 against rVSV MERS-CoV are graphed.

FIG. 12A is a graph of the neutralization potency of the bovine HIV-1 IgG NC-Cowl and ODIN derivatives. NC-Cowl IgG and ODIN-8 are used as positive and negative controls, respectively. ODIN-5, ODIN-6, and ODIN-9 demonstrate the ability to actively neutralize HIV-1 pseudovirus.

FIG. 12B is a graph of the neutralization potential of ODIN-5 is compared to two different ODIN-5 Fc fusions with different hinge region sequences, ODIN-5 Fc V1 and ODIN-5 Fc V2.

FIG. 13A is a graph showing the neutralization potency of the wild-type ADI-15878 IgG, ADI-15878 scFv, and ADI-15878 scFv-Fc against rVSV-EBOV is graphed to establish a baseline prior to ODIN based modifications.

FIG. 13B is a graph depicting the neutralization potency of ODIN-10 and an ODIN-10 Fc region fusion, ODIN-10 Fc V1, are shown compared to wild-type ADI-15878 IgG.

FIG. 13C is a graph depicting the neutralization potency of ODIN-11 and an ODIN-11 Fc region fusion, ODIN-11 Fc V1 are shown compared to wild-type ADI-15878 IgG.

FIG. 14A is a graph of bio-layer interferometry (BLI) binding kinetics of ODIN-10 to the HIV-1 BG505 spike protein at multiple analyte concentrations: 1500 nM, 750 nM, 375 nM, and 187.5 nM. Association is to the left of the vertical dashed line and dissociation is show to the right of the vertical dashed line.

FIG. 14B is a graph showing the BLI binding kinetics of ODIN-11 to the HIV-1 BG505 spike protein at multiple analyte concentrations: 1500 nM, 750 nM, 375 nM, and 187.5 nM. Association is to the left of the vertical dashed line and dissociation is show to the right of the vertical dashed line.

FIG. 14C is a graph of the neutralization potency of ODIN-10 and ODIN-11 is graphed in comparison to ODIN-5, ODIN-8 (negative control) and NC-Cowl IgG (positive control).

FIG. 15A is a graph depicting bio-layer interferometry (BLI) data for the binding of epidermal growth factor receptor (EGFR) and SARS-CoV-2 spike by ODIN-8 showing VHH-72 has no affinity to EGFR.

FIG. 15B is a graph depicting bio-layer interferometry (BLI) data for the binding of epidermal growth factor receptor (EGFR) and SARS-CoV-2 spike by ODIN-8 showing VHH-72 has high affinity to the SARS-CoV-2 spike.

FIG. 15C is a bio-layer interferometry (BLI) graph showing ODIN-8 has a high affinity to EGFR, in contrast to the parental VHH-72 shown in FIG. 15A.

FIG. 15D is a BLI graph showing ODIN-8 has a high affinity to the SARS-CoV-2 spike protein.

FIG. 16A is a diagram of a sequence inserted into VHH-72 is shown in bold with the NST glycan site underlined and labeled. The flanking VHH-72 sequence is shown on either side of the insertion sequence.

FIG. 16B is SDS-PAGE results of the VHH-72/Gly protein was comparatively analyzed with wild-type VHH-72. A glycan site (GSSGNSTGSSG) was inserted into VHH-72 via the C-C′ loop insertion site in FR2. The proteins were exposed to PNGase F under native or denaturing conditions. An untreated sample of VHH-72/Gly shows a clear gel shift resulting from active glycosylation when compared to VHH-72. Following PNGase F exposure, the glycan is removed from the protein and VHH-72 and VHH-72/Gly migrate similarly, confirming the shift is the result of active glycosylation at the modified insertion site.

### DETAILED DESCRIPTION

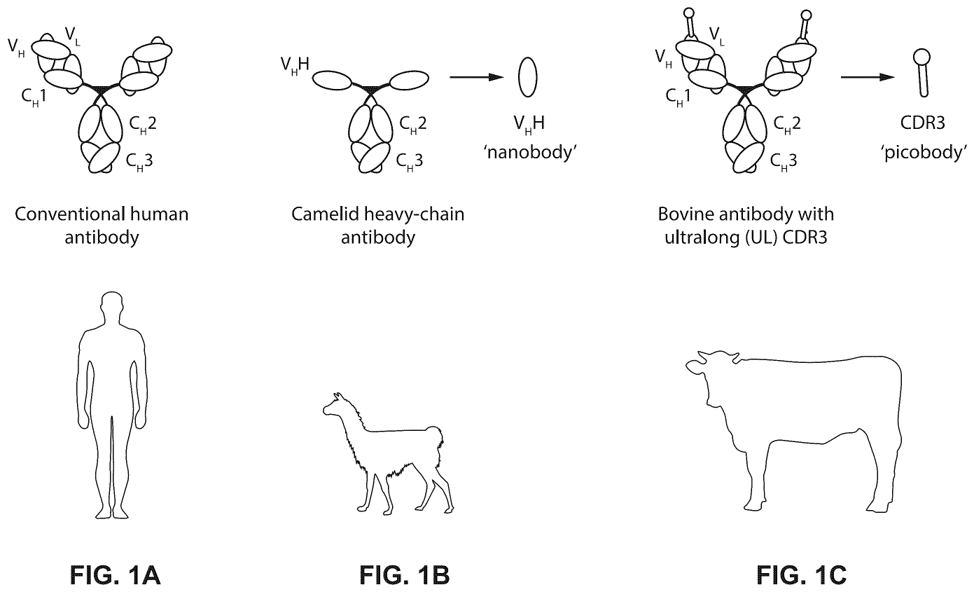

Referring more specifically to the drawings, for illustrative purposes, systems and methods for producing and using a platform for engineered antigen-binding proteins (e.g., bispecific antigen-binding proteins, e.g., single-domain bispecifics) called ODIN (Orthogonal Dual-Interacting Nanotherapeutics) are generally shown. The platform is useful in generating a wide range of engineered antigen-binding proteins. Several embodiments of the technology are described generally in FIG. 1A to FIG. 16B to illustrate the characteristics and functionality of the constructs, systems, and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology. Although a specific device architecture is used to illustrate certain constructs, other structures and adaptations can be used to achieve the functional compositions and diagnostic or therapeutic methods.

Immunotherapeutics are a mainstay in the treatment of cancer, autoimmune diseases, neurological disorders and infectious diseases, accounting for ˜70% of all biopharmaceuticals sales in recent years. In 2020, the global market for antibody drug products approached $160B and is expected to eclipse $300B by 2025. Although this market is largely driven by monoclonal antibodies (mAbs; FIG. 1A), the large size (˜150 kDa) and complexity of mAbs has imposed high development and manufacturing costs and limited their therapeutic utility and global access.

Consequently, the field has begun to explore alternative biologic platforms that overcome these liabilities. Single-domain antibodies (sdAbs), including the ˜20 kDa camelid VHH or ‘nanobody’, illustrated in FIG. 1B, represent one such new but rapidly growing treatment modality, with one FDA-approved VHH and >20 others in clinical trials to treat a variety of solid and hematological cancers, autoimmune diseases, neurological disorders and bacterial, fungal, and viral infections (Table 1).

Ultralong variable CDR3s elaborated by a subset of bovine mAbs and comprising a beta-hairpin stalk and a disulfide-rich knob domain provide an even smaller (˜10 kDa) autonomous antigen-binding structure, the ‘picobody’ illustrated in FIG. 1C), that appears ideally suited to insert into cryptic sites in target proteins and is beginning to garner interest.

In certain aspects, provided herein are antigen-binding proteins comprising an FR2 region of a VH, VL, or VHH domain; or an FW2/HV2 region of a VNAR domain e.g., FR2 REGION OF VH, VL, OR VHH

### A. FR2 Region of VH, VL, or VHH

Conventional antibodies comprise variable domains such as VH and VL domains. A heavy-chain antibody, which is found in e.g., camelid, which comprises two heavy chains without the two light chains that are usually found in the conventional antibodies. The heavy-chain antibody from camelid comprises a variable domain known as a VHH domain. Despite overall differences, the variable domains including VH, VL, and VHH domains comprise the following common structure:
FR1-CDR1-FR2-CDR2-FR3-CDR3-FR4
   [as present in variable heavy (VH), variable light (VL), VHH (e.g., in camelid)] FR: framework region; CDR: complementarity-determining region.

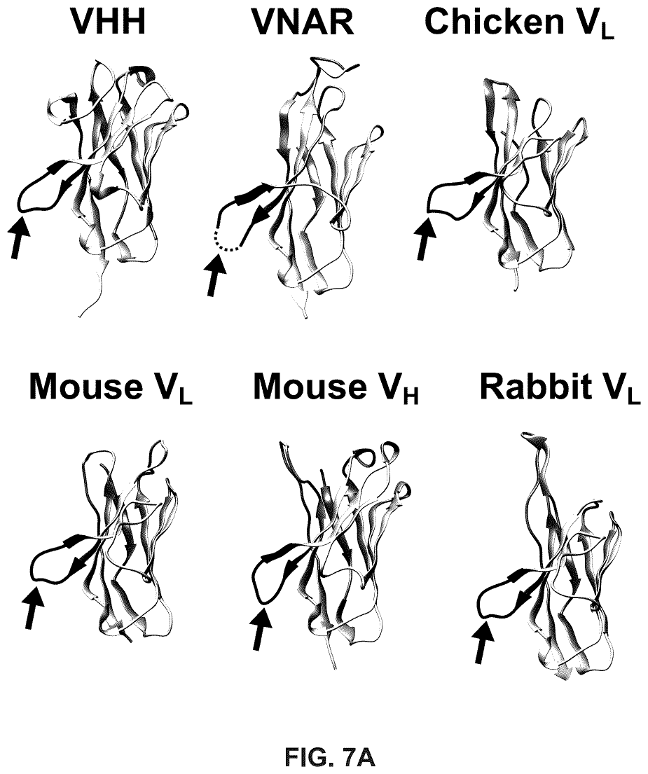

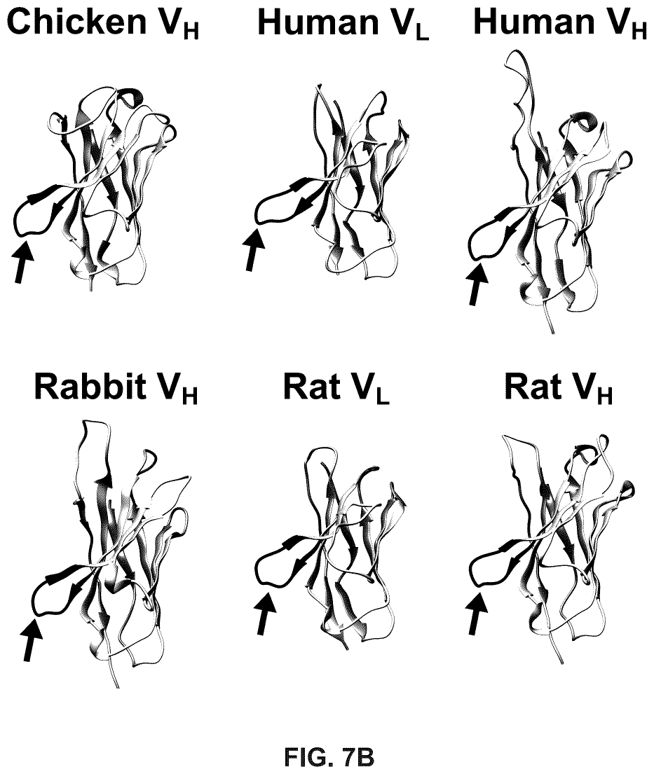

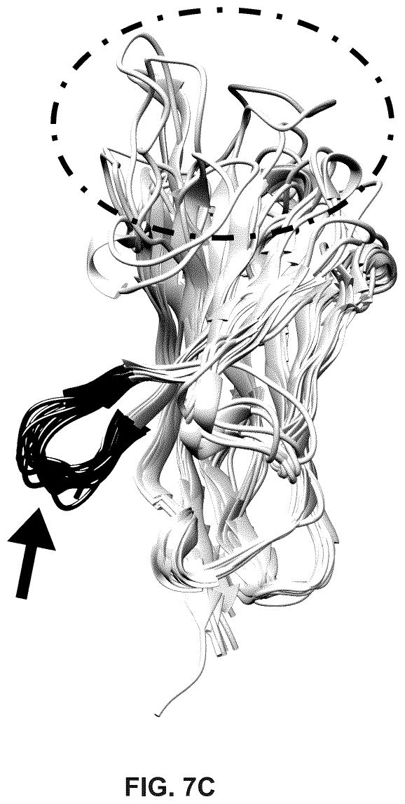

The FR2 region of the variable domains (e.g., VH, VL, or VHH domain) is conserved in structure within and across species as illustrated in FIG. 7.

The FR2 region is defined as the amino acid residues 39-55 of the V-domains and V-like domains (e.g., VH, VL, or VHH domain) according to the IMGT numbering system (see e.g., Table 2).

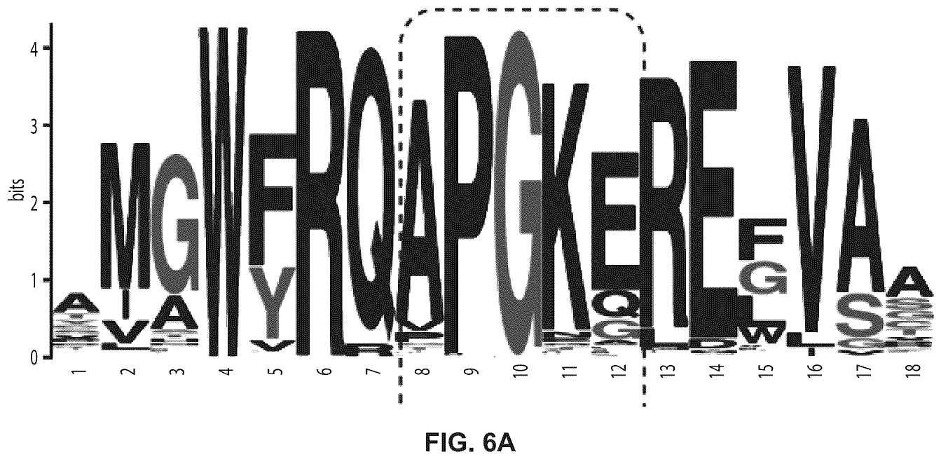

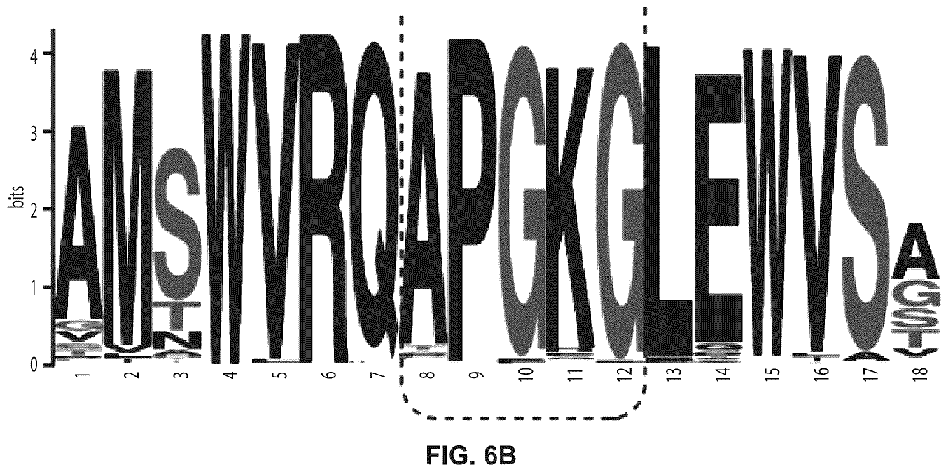

Representative FR2 sequences of the VHH, VH, and VL domains in various species are shown in Table 3 through Table 6. It can be seen that the sequences of the FR2 region in VH, VL, and VHH are conserved within a species (see e.g., the degenerate sequences representing FR2 of VHH as illustrated in FIG. 6A, and FR2 of human VH3-23 germline are shown in FIG. 6B. Tables 4-6 illustrate the publicly available representative sequences of FR2 in various species as well as the similarity/conservation of such sequences. Sequences are identified by their PDB ID. FR2 sequences correspond to residues 39-55 based on IMGT nomenclature.

In certain aspects, provided herein are antigen-binding proteins comprising an FR2 region of a VH, VL, or VHH domain; or an FW2/HV2 region of a VNAR domain.

### B. FW2/HV2 Region of VNAR

Similar to camelids, cartilaginous fishes (e.g., shark) also have heavy-chain antibodies that lack light chains. These heavy-chain antibodies are called IgNAR, “immunoglobulin new antigen receptor.” The IgNAR comprises a variable domain, which is the variable domain of new antigen receptor (VNAR). VNAR domains comprise the following structure:
FW1-CDR1-FW2-HV2-FW3a-HV4-FW3b-CDR3-FW4
[as present in Shark VNAR]
FW: framework region; CDR: complementarity-determining region
HV: hypervariable region

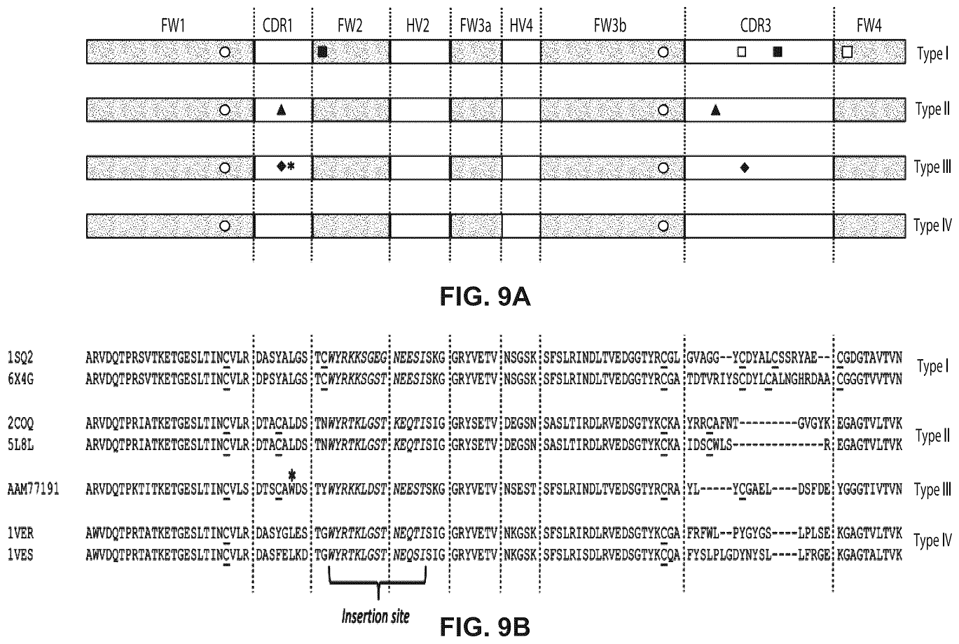

Four representative types of VNAR domains and their sequences are shown in FIG. 9. Additional information regarding VNARs or their FW2 regions is known in the art. As used herein, the FW2/HV2 region (as demarcated in FIG. 9A and FIG. 9B) comprises 19 amino acids, labeled as the amino acid residues 1-19 (FW2 region comprising the amino acid residues 1-11; and HV2 region comprising the amino acid residues 12-19; see FIG. 9A and FIG. 9B.

### C. Exemplary Insertion Sites

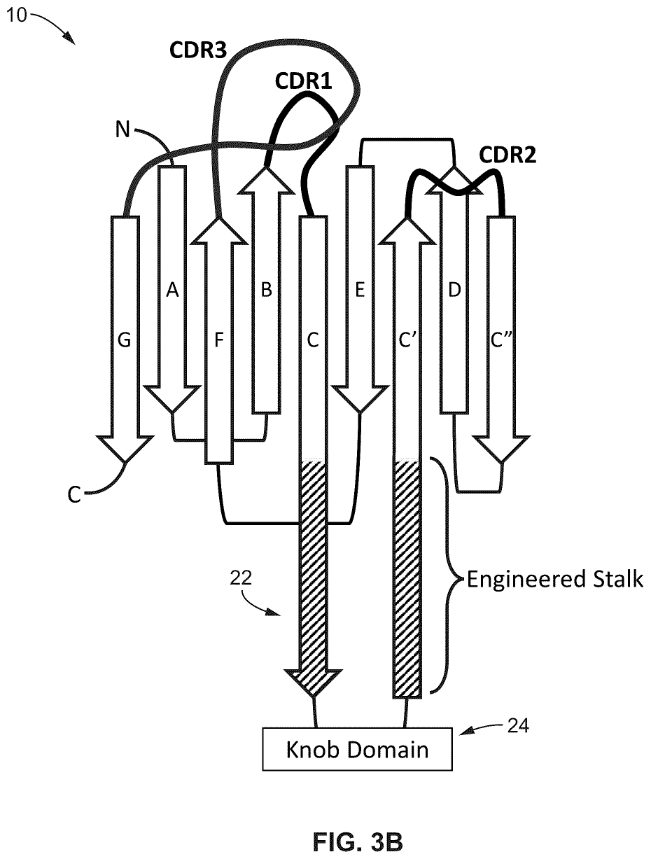

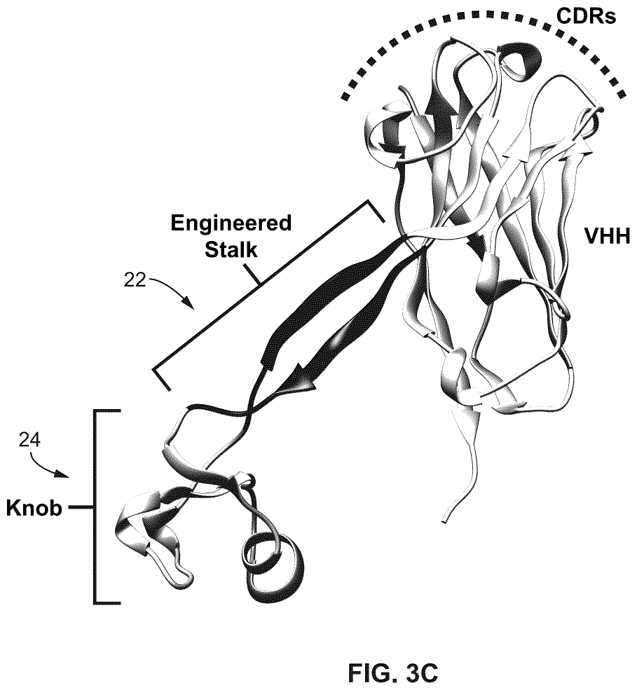

While seemingly different, the FR2 region of VH, VL, and VHH; and the FW2/HV2 region of VNAR surprisingly share structural similarities. Specifically, the FR2 and FW2/HV2 regions comprise a β hairpin having a β strand-loop-β strand structure (see FIG. 3A through FIG. 3C and FIG. 7). As can be seen in Fig. FIG. 7, the β strands of the FR2 and FW2/HV2 region protrude outwardly such that the loop region points away from the VH, VL, VHH, or VNAR domain.

As discovered herein, the loop region of the β hairpin is amenable for insertion of a heterologous polypeptide, without interfering with the function of either the heterologous polypeptide or the antigen-binding protein comprising the FR2 or FW2/HV2 region.

One ODIN single-domain bispecific design 10 is shown schematically in FIG. 3A. The structural organization of VHH has interconnected beta sheets 12 and complementarity-determining regions (CDR1) 14, (CDR2) 16 and (CDR3) 18 that are involved in antigen recognition are shown. The framework structure also has an insertion site 20 that is indicated with a dashed line (between beta sheets C and D). The insertion site 20 allows the insertion of selected heterologous polypeptides into the structure. A tandem VHH fusion with an engineered stalk 22 and knob 24 structure is shown schematically in FIG. 3B. FIG. 3C shows a structural model of the ODIN VHH-picobody that has been engineered to display bovine ‘stalk and knob’ picobody.

In certain aspects, provided herein is an FR2 region of a VH, VL, or VHH domain comprising a heterologous polypeptide, wherein the FR2 region comprises a deletion of at least 0, 1, or 2 amino acids.

In some embodiments, the FR2 region comprises a deletion of no more than 10, 9, 8, 7, 6, 5, 4, 3, or 2 amino acids. In some embodiments, the FR2 region comprises a deletion of at least 5 amino acids and no more than 6 amino acids. In some embodiments, the FR2 region comprises a deletion of 5 amino acids, optionally wherein the deletion comprises the amino acids 45-49 of the FR2 region (IMGT numbering). In some embodiments, the FR2 region comprises a deletion of 6 amino acids, optionally wherein the deletion comprises the amino acids 44-49 of the FR2 region (IMGT numbering). In some embodiments, the deletion of the amino acid(s) is within the amino acids 44-49 of the FR2 region (IMGT numbering). In some embodiments, the heterologous polypeptide is inserted immediately C-terminal to the amino acid 43, 44, 45, 46, 47, 48, or 49 of the FR2 region (IMGT numbering), optionally wherein the heterologous polypeptide is inserted immediately C-terminal to the amino acid 43 or 44. In some embodiments, the FR2 region is of a human, a camelid, or a humanized VH, VL, or VHH domain.

In certain aspects, provided herein is an FW2/HV2 region of a VNAR domain comprising a heterologous polypeptide, wherein the FR2 region comprises a deletion of at least 0, 1, or 2 amino acids.

In some embodiments, the FW2/HV2 region comprises a deletion of no more than 14, 12, 10, 8, or 6 amino acids. In some embodiments, the FW2/HV2 region comprises a deletion of at least 5 amino acids and no more than 14 amino acids. In some embodiments, the FW2/HV2 region comprises a deletion of 14 amino acids, optionally wherein the deletion comprises the amino acids 3-16 of the FW2/HV2 region. In some embodiments, the deletion of the amino acid(s) is within the amino acids 3-16 of the FW2/HV2 region. In some embodiments, the heterologous polypeptide is inserted immediately C-terminal to the amino acid 2, 3, 4, 5, 6, 7, 8, 9, 10, 11, 12, 13, 14, or 15 of the FW2/HV2 region. In some embodiments, the FW2/HV2 region is of a shark or a humanized VNAR.

The structural contexts of the FR2 or the FW2/HV2 region are common to the orthogonal domains across species (see e.g., camel, mouse, rabbit, goat, shark, horse, chicken, hamster, human, and the like; see FIG. 7). Accordingly, the FR2 or the FW2/HV2 region of all species can be engineered to comprise a heterologous polypeptide. Notably, many FDA-approved biologics (e.g., antibodies or fragments thereof, antibody-drug conjugates (ADCs), bispecifics, BITEs, etc.) comprise the FR2 region of the present disclosure. Representative FDA-approved antibodies are presented in Tables 7 through 10.

In Table 7, FR2 sequences from variable heavy (VH) and variable light (VL) domains from approved whole mAbs are presented. Sequences were retrieved from the Thera-SAbDab database, which is populated by molecules from the World Health Organization Proposed International Nonproprietary Names (WHO INN) List 126. The FR2 sequences from the indicated VH or VL correspond to residues 39-55 based on IMGT nomenclature.

Sequences from variable heavy (VH) and variable light (VL) domains from approved mAb conjugates are presented in Table 8. Sequences were retrieved from the Thera-SAbDab database, which is populated by molecules from the WHO INN List 126. The FR2 sequences from the indicated VH or VL correspond to residues 39-55 based on IMGT nomenclature. Conjugates include antibody-drug conjugates (ADCs), fusions to an additional protein, or radiolabeled mAbs.

FR2 sequences from variable heavy (VH) and variable light (VL) domains from approved alternative mAb formats are illustrated in Table 9. Sequences were retrieved from the Thera-SAbDab database, which is populated by molecules from the WHO INN List 126. The FR2 sequences from the indicated VH or VL correspond to residues 39-55 based on IMGT nomenclature. Alternative formats include Fab, Fv fusions or scFv.

In Table 10, FR2 sequences from variable heavy (VH) and variable light (VL) domains from approved bispecific antibody formats are provided. Sequences were retrieved from the Thera-SAbDab database, which is populated by molecules from the WHO INN List 126. The FR2 sequences from the indicated VH or VL correspond to residues 39-55 based on IMGT nomenclature. Bispecific formats include bispecific mAbs or BITEs (bispecific T cell engager).

### D. Representative Heterologous Polypeptides

As used herein, the heterologous polypeptide is not limited by its size (see Definitions). In certain aspects, provided herein is a heterologous polypeptide comprising one or more amino acids. In some embodiments, the heterologous polypeptide comprises one amino acid. In some embodiments, the heterologous polypeptide comprises at least 2 amino acids and no more than 50 amino acids (e.g., peptide, e.g., a linker (e.g., 4 amino acid-long or 8 amino acid-long linkers as presented herein). In some embodiments, the heterologous polypeptide comprises at least 51 amino acids.

In some embodiments, the heterologous polypeptide comprises an oligomerization domain. In some embodiments, the oligomerization domain is selected from GCN4 leucine zipper, phage T4 fibritin foldon domain, Comp48, and engineered oligomeric beta sheet. In some embodiments, the heterologous polypeptide comprises a cytokine or a chemokine, optionally wherein the cytokine or the chemokine comprises IL-2, IL-10, IL-12, IL-15, IL-18, IL-21, GM-CSF, TNF-α, IFN-α, IFN-β, or IFN-γ. In some embodiments, the heterologous polypeptide comprises PD-1, PD-L1, CTLA-4, B7, or CD3.

In some embodiments, the heterologous polypeptide comprises a detectable marker, optionally wherein the detectable marker is a fluorescent protein (e.g., GF P or a derivative thereof), or a peptide tag (e.g., a histidine tag (e.g., 8×HIS), a hemagglutinin tag (HA tag; amino acid sequence YPYDVPDYA), a flag tag (amino acid sequence DYKDDDDK), a myc tag (amino acid sequence EQKLISEEDL), a strep tag (WSHPQFEK), and/or an A56R protein).

In some embodiments, the heterologous polypeptide comprises an enzyme, optionally wherein the enzyme is a luciferase. In some embodiments, the heterologous polypeptide comprises a polymer (e.g., polyethylene glycol (PEG)) or a polypeptide that extends the serum's half-life. In some embodiments, the polypeptide that extends the serum half-life is selected from an albumin-binding protein, an anti-albumin antibody or a fragment thereof (e.g., CA645), albumin (e.g., human serum albumin), an immunoglobulin, an Fc domain, a fragment of an Fc domain, and an FcRnBP. PEGylation via site-specific conjugation is known in the art. Exemplary methods of extending the serum half-life are also known in the art.

In some embodiments, the heterologous polypeptide comprises at least one natural or unnatural amino acid. In some embodiments, at least one natural amino acid comprises a cysteine, cystine, tyrosine, serine, threonine, lysine, and/or histidine. In some embodiments, the at least one unnatural amino acid comprises an unnatural amino acid comprising an azide, alkynes, an aldehyde, an aminooxy, a functionalized arene, or a trans-cyclooctene (e.g., for bio-orthogonal labeling); fluorosulfate-L-tyrosine (FSY); L-Azidohomoalanine hydrochloride; L-Azidonorleucine hydrochloride; p-acetylphenylalanine (pAcPhe); para-acetylphenylalanine (pAF); para-azidophenylalanine (pAZ); N6-((2-azidoethoxy)carbonyl)-I-lysine; a cysteine and selenocysteine derivative; a leucine derivative; a phenylalanine derivative; a lysine derivative; a tryptophan derivative; and/or a tyrosine derivative.

In some embodiments, the heterologous polypeptide comprises a glycosylation site, wherein the glycosylation site comprises an amino acid sequence of NXT or NXS, wherein X is any amino acid except proline. In some embodiments, the heterologous polypeptide is glycosylated and comprises a glycan. Engineered glycosylation sites and site-specific conjugation at glycan molecules are well known in the art.

In some embodiments, the least one natural or unnatural amino acid, or the glycan is conjugated. In some embodiments, the at least one natural or unnatural amino acid, or the glycan is conjugated to polyethylene glycol (PEG), a chemotherapeutic agent, and/or a cytotoxic agent.

In some embodiments, the heterologous polypeptide comprises a linker, optionally wherein the linker comprises the amino acid sequence of GSSG or GSSGSSG. In some embodiments, the linker is longer.

In some embodiments, the heterologous polypeptide comprises an antigen-binding protein. Such antigen-binding protein can be any one of the antigen-binding proteins described herein (see below) or those known in the art.

In some embodiments, the antigen-binding protein comprises any one of the antigen-binding proteins listed in Tables 7 through 10, or a fragment thereof.

In some embodiments, the antigen-binding proteins of the present technology bind an antigen that is in systemic circulation (e.g., cytokines, toxins).

In some embodiments, the antigen-binding protein binds an antigen expressed on a virus, a cancer cell, a neuron, a motor neuron, and/or an immune cell (e.g., of a healthy subject or a diseased subject or a cancer cell line). In preferred embodiments, the antigen-binding proteins bind an antigen that is overexpressed on a diseased cell or a cell from a diseased subject relative to a healthy cell or a cell from a healthy subject. In preferred embodiments, the antigen is overexpressed on cancer cell relative to a healthy cell.

In some embodiments, the antigen-binding protein comprises an ultralong CDR3 (UL-CDR3), scFV, Fab′, F(ab′)2, ds-scFv, scFab′, diabody, scFV-CH3 (minibody), a VHH domain, a VH domain, a VL domain, or a VNAR domain. In preferred embodiments, the heterologous polypeptide is a bovine ultralong CDR3 (UL-CDR3).

### E. Representative ODIN Molecules

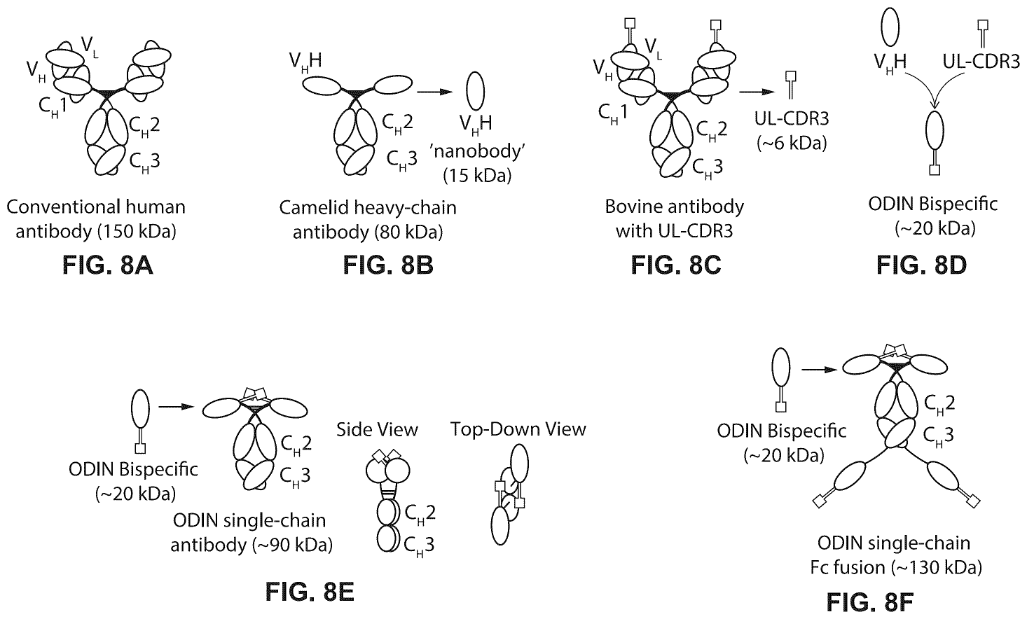

Various types of ODIN molecules can be generated by combining any antigen-binding protein/domain with a heterologous polypeptide. In some embodiments, the ODIN molecules are bispecific or biparatopic molecules comprising two antigen-binding moieties that bind different antigens/epitopes. In some embodiments, the ODIN molecules are multi-specific (see examples in FIG. 8.

In certain aspects, provided herein is a VH domain comprising the FR2 region, which comprises a heterologous polypeptide of the present disclosure.

In certain aspects, provided herein is a VL domain comprising the FR2 region, which comprises a heterologous polypeptide of the present disclosure.

In certain aspects, provided herein is a VHH domain comprising the FR2 region, which comprises a heterologous polypeptide of the present disclosure.

In certain aspects, provided herein is a VNAR domain comprising the FW2/HV2 region, which comprises a heterologous polypeptide of the present disclosure.

In certain aspects, provided herein is an antigen-binding protein comprising the FR2 region or FW2/HV2 region of the present disclosure. In certain aspects, provided herein is an antigen-binding protein comprising the VH domain of the present disclosure. In certain aspects, provided herein is an antigen-binding protein comprising the VL domain of the present disclosure. In certain aspects, provided herein is an antigen-binding protein comprising the VHH domain of the present disclosure. In certain aspects, provided herein is an antigen-binding protein comprising the VNAR domain of the present disclosure.

In some embodiments, the antigen-binding protein comprises an Fc domain.

In some embodiments, the antigen-binding protein comprises at least two of the FR2 region or FW2/HV2 region.

In some embodiments, the antigen-binding protein comprises (a) any two of the FR2 region or FW2/HV2 region and (b) an Fc domain, wherein the any two of the FR2 region or FW2/HV2 region are fused to the N-terminus of the Fc domain.

In some embodiments, the antigen-binding protein comprises at least four of the FR2 region or FW2/HV2 region.

In some embodiments, the antigen-binding protein comprises (a) any four of the FR2 region or FW2/HV2 region and (b) an Fc domain, wherein the two of the FR2 region or FW2/HV2 region are fused to the N-terminus of the Fc domain, and the other two of the FR2 region or FW2/HV2 region are fused to the C-terminus of the Fc domain.

In some embodiments, the antigen-binding proteins of the present disclosure further comprises a detectable marker or a peptide tag. In some such embodiments, the detectable marker or the peptide tag may be selected from a GF P or a derivative thereof, a histidine tag (e.g., 8×HIS), a hemagglutinin tag (HA tag; amino acid sequence YPYDVPDYA), a flag tag (amino acid sequence DYKDDDDK), a myc tag (amino acid sequence EQKLISEEDL), a strep tag (WSHPQFEK), and an A56R protein.

In some embodiments, the antigen-binding proteins of the present disclosure further comprises an enzyme, e.g., luciferase.

In some embodiments, the antigen-binding proteins of the present disclosure further comprises a polymer (e.g., polyethylene glycol (PEG)) or a polypeptide that extends the serum's half-life. In some such embodiments, the polypeptide that extends the serum half-life may be selected from an albumin-binding protein, an anti-albumin antibody or a fragment thereof (e.g., CA645), albumin (e.g., human serum albumin), an immunoglobulin, an Fc domain, a fragment of an Fc domain, and an FcRnBP.

In some embodiments, the antigen-binding protein comprises any one of the antigen-binding proteins listed in Tables 7-10, or a fragment thereof.

In some embodiments, the antigen-binding proteins of the present disclosure bind an antigen that is in systemic circulation (e.g., cytokines, toxins).

In some embodiments, the antigen-binding proteins of the present disclosure bind an antigen expressed on a virus, a cancer cell, a neuron, a motor neuron, and/or an immune cell (e.g., of a healthy subject or a diseased subject or a cancer cell line). In preferred embodiments, the antigen-binding proteins bind an antigen that is overexpressed on a diseased cell or a cell from a diseased subject relative to a healthy cell or a cell from a healthy subject. In preferred embodiments, the antigen is overexpressed on cancer cell relative to a healthy cell.

In certain aspects, provided herein is a chimeric antigen receptor (CAR) comprising the FR2 region or FW2/HV2 region of the present disclosure (e.g., the FR2 or FW2/HV2 region comprising a heterologous polypeptide). In certain aspects, provided herein is a chimeric antigen receptor comprising the VH domain, VL domain, VHH domain, or VNAR domain comprising a heterologous polypeptide as described herein.

In some embodiments, the chimeric antigen receptor binds at least two antigens (e.g., dual CAR).

Various CAR therapies (e.g., CAR-T, CAR-M, CAR-NK, and dual CARs) are contemplated. Accordingly, various immune cells may comprise the CAR molecules described above, e.g., T cells, macrophages, NK cells, etc. Examples of certain beneficial ODIN molecules are provided below.

1. Single-Domain Antibodies (sdAbs)

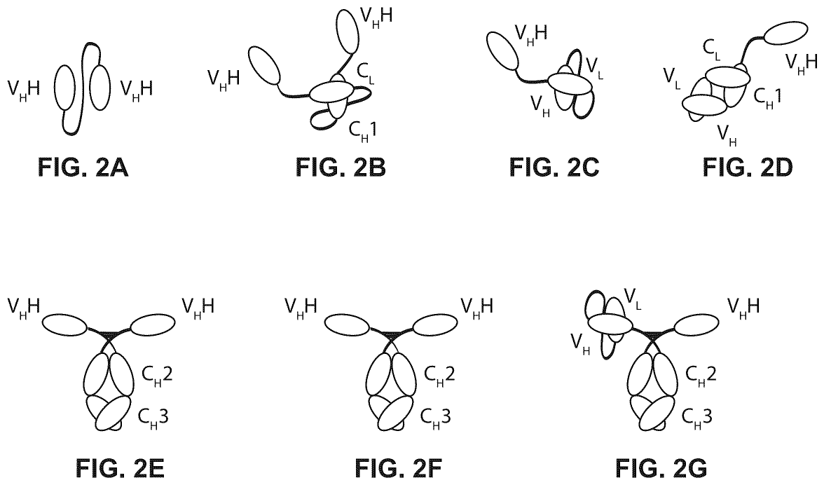

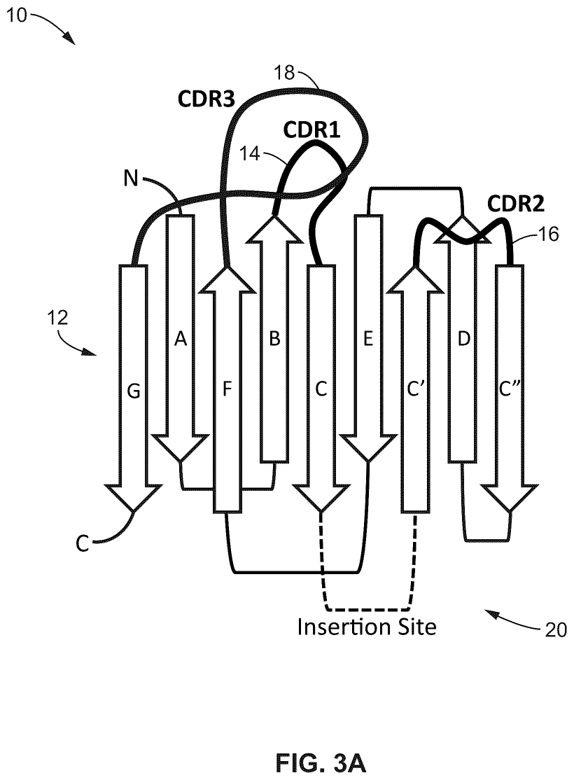

In some embodiments, the engineered antigen-binding proteins are sdAbs. Although sdAbs embody the idea that desirable features (e.g., enhanced stability and solubility, rapid biodistribution, low-cost manufacturing), come in little packages, they also suffer key disadvantages relative to mAbs. These disadvantages include: an exceptionally short half-life, a lack of avidity, and the inability to induce mAb Fc-mediated effector functions. Two conventional strategies to equip sdAbs with these additional functions are to daisy-chain sdAbs with flexible linkers (FIG. 2A to FIG. 2D) or fuse them to antibody Fc sequences (See FIG. 2E through FIG. 2G). These additional binding domains reduce drug clearance and extend half-life, provide avidity and mechanistic synergy, and/or bring two or more host targets or cells together to effect therapeutic activity. However, both approaches to engineer bispecific sdAbs have liabilities: flexible inter-domain linkers reduce drug stability in vitro and can be susceptible to cleavage and loss of activity in vivo, and the addition of a bulky and complex Fc domain erodes the very advantages sought from these alternative platforms.

As described herein, sdAbs prepared using the ODIN platform technology address many of these liabilities by affording bispecific activity in a small (20-25 kDa) protein with a single well-folded domain-one of the smallest such molecules engineered to date. ODIN's small size affords exceptionally high biodistribution rates, critical for therapeutics targeting toxins, pathogens, solid cancerous tumors, and other disease targets requiring tissue penetration. Further, ODIN therapeutics can be manufactured in alternative non-mammalian cell-based systems (e.g., bacteria or yeast), significantly reducing costs and time to Phase I clinical trials. Finally, the unique capacity of the bovine ‘stalk-and-knob’ picobody domains to insert into narrow clefts and pockets in proteins will shrink ODIN's steric footprint even further and enable the targeting of cryptic sites and quaternary structures beyond the reach of classical scFv or IgG1-based bispecifics.

There are other antibody fragment technologies currently on market or in advanced development engineered for smaller size and multi-specificity, including the bispecific T-cell engager (BiTE), dual affinity re-targeting proteins (DARTs) and Tandem diabodies (TandAbs). However, in contrast to ODIN of the present disclosure, these are multi-domain molecules fused together via engineered linkers, making them complex molecules to manufacture, and often requiring expression in mammalian cell systems such as Chinese hamster ovary cells (CHO). Sanofi (formerly Ablynx) has also pursued bispecific “nanobodies” (camelid VHHs), which are simply two nanobodies fused together via an engineered linker, as described above. Molecules daisy-chained together via linkers may suffer from biophysical instability, which impacts their pharmacological properties, introduces manufacturing liabilities, and reduces their shelf-life, as has been observed for a number of these products. In contrast, the ODIN bispecific VHHs are predicted to be extremely stable and cheaper to manufacture. Moreover, they leverage two naturally occurring yet divergent immune repertoires (camelid and bovine) in a manner that allows ODIN bispecifics to target challenging epitopes on target antigens more readily than current technologies.

### 2. CAR Molecules and CAR Therapy

The engineered antigen-binding proteins of the present disclosure (e.g., ODIN molecules) can be used anywhere an antibody or its derivatives are used. One exemplary area of utility is a CAR therapy. Chimeric antigen receptors (CARs) are transmembrane proteins that have been engineered to give the cells (e.g., T cells, macrophages, NK cells) the new ability to target/bind a specific protein. The receptors are chimeric because they combine both antigen-binding and certain cellular functions (e.g., T cell activating function) into a single receptor. For example, the receptor can comprise an extracellular antigen-binding domain (e.g., scFv) that binds to a specific antigen (e.g., those highly and specifically expressed on the surface of cancer cells) fused to a transmembrane domain and an intracellular costimulatory domain/activation domain.

The engineered antigen-binding proteins of the present disclosure can be used as the extracellular antigen-binding domain of the CAR molecule. In some embodiments, such a domain is a biparatopic domain, which comprises two antigen-binding domains that can bind to two different epitopes on a single antigen. Such biparatopic domains can increase the specificity and avidity of binding such that it can elicit a stronger CAR response. In other embodiments, such a domain is a bispecific domain, which comprises two antigen-binding domains that can bind to two different antigens. Such domains can target two antigens, e.g., on a cancer cell, thereby increasing the specificity (and presumably safety) of the binding and activation of the immune cells in which they are expressed. These domains have proven to be effective especially in a recurrence or refractory disease in which the target cell (e.g., cancer cell) loses expression of one antigen. The dual CARs are described further (see below).

### 3. CAR T Therapy

Chimeric antigen receptor T cells (CAR T cells) are T cells that are engineered to express the CAR proteins for cancer therapy. CARs enable T cells to recognize tumor-associated antigens (TAAs) in a major histocompatibility complex (MHC)-independent manner. CAR T therapy can use T cells that are autologous or allogeneic to the patient. After CAR T cells are infused into a patient, they act as a “living drug” against cancer cells. When they come in contact with their targeted antigen on a cell, CAR T cells bind to it and become activated, then proceed to proliferate and become cytotoxic. CAR T cells destroy cells through several mechanisms, including extensive stimulated cell proliferation, increasing the degree to which they are toxic to other living cells (cytotoxicity) and by causing the increased secretion of factors that can affect other cells such as cytokines, interleukins and growth factors. The first CAR T cell therapies were FDA-approved in 2017, and there are now 6 approved CAR T therapies.

There are several variations/generations of CAR designs. The first reports of tumor-targeting CARs demonstrated that an scFv recognizing antigens such as human epidermal growth factor receptor 2 (HER2) fused to the CD3ζ signaling domain can elicit tumor-specific cytotoxicity, but T cells expressing these “first-generation” CARs that included only the CD3ζ chain for T-cell signaling generally failed to elicit potent antitumor effects. In the following years, second- and third-generation CARs emerged that included one or two costimulatory domains, respectively, drawing from the biological understanding that the endogenous TCR requires association with other costimulatory or accessory molecules for robust signaling. Most commonly derived from CD28 or 4-1BB, these costimulatory domains conferred more potent antitumor cytotoxicity, increased cytokine production, and improved proliferation and persistence of CAR-T cells.

The choice of costimulatory domain has an impact on a wide range of properties, including metabolic pathways, T-cell memory development, and antigen-independent tonic signaling, prompting further research into other costimulatory domains. For example, a third-generation CAR with OX40 and CD28 costimulatory domains repressed CD28-induced secretion of interleukin (IL)-10, an anti-inflammatory cytokine that compromises T-cell activity. In addition, the inducible T-cell co-stimulator (ICOS) costimulatory domain in combination with either CD28 or 4-1BB co-stimulation increased in vivo persistence, and MyD88/CD40 co-stimulation improved in vivo proliferation of CAR-T cells.

More recently, fourth-generation CARs that incorporate additional stimulatory domains, commonly referred to as “armored” CARs, have been reported. In one example, the engineered armored CAR-T cells termed “T cells redirected for universal cytokine-mediated killing” (TRUCK) have been engineered to secrete the proinflammatory cytokine IL-12 to stimulate innate immune cells against the tumor and resist inhibitory elements of the TME, including regulatory T (Treg) cells and myeloid-derived suppressor cells (MDSCs). The secretion of other soluble factors has been studied, including IL-15 or IL-18 to enhance T cell proliferation, as well as the combination of CCL19 and IL-7 to recruit endogenous immune cells and establish a memory response against tumors.

Accordingly, the engineered antigen-binding proteins of the present disclosure can be incorporated into any variations or generations of the CAR T cells for use as e.g., a cancer therapy.

### 4. Dual CAR Therapy

CAR T cells with ability to target two antigens on a cancer cell surface have been proven to be effective clinically. For example, CAR T cells with dual targeting of CD19 and CD22 in adult patients with recurrent or refractory B cell malignancies showed improved efficacy.

In addition, dual CAR T demonstrated effectiveness in targeting tumor cells with heterogeneous antigen expression. For example, CAR-T cells targeting simultaneously two tumor-associated antigens with trans-acting CD28 and 4-1BB co-stimulation caused rapid antitumor effects in in vivo stress conditions, protection from tumor re-challenge and prevention of tumor escape due to low antigen density. Molecular and signaling studies indicated that T cells engineered with the dual CAR design demonstrated sustained phosphorylation of T-cell-receptor-associated signaling molecules and a molecular signature supporting CAR-T-cell proliferation and long-term survival. Furthermore, metabolic profiling of CAR-T cells displayed induction of glycolysis that sustains rapid effector T-cell function, but also preservation of oxidative functions, which are critical for T-cell long-term persistence.

### 5. CAR-M Therapy

Programming CARs into cell types other than T cells can further expand the versatility of the therapy by realizing new functions unachievable by CAR T cells. It was recently demonstrated that primary macrophages can be engineered with CARS via adenoviral transduction. The resulting CAR M cells exhibited tumor-specific phagocytosis, inflammatory cytokine production, polarization of bystander macrophages to the immunostimulatory M1 phenotype, and cross-presentation of the tumor associated antigen (TAA) to bystander T cells.

### 6. CAR-NK Therapy

CD19-targeting CAR-NK cells have achieved robust clinical efficacy without inducing cytokine release syndrome (CRS), neurotoxicity, or graft-versus-host syndrome (GvHD) in patients with B-cell lymphoid tumors. CAR NK cells have been shown to exert potent and specific cytotoxicity toward a variety of tumor models, including leukemia, multiple myeloma, ovarian cancer, and glioblastoma, as well as toward immunosuppressive cell types such as myeloid-derived suppressor cells (MDSCs) and follicular helper T cells (TFH). Lastly, natural killer T (NKT) cells possess antitumor and tumor-homing capabilities, and GD2-targeting CAR NKT cells that harness these inherent advantages exhibited effective localization to and lysis of neuroblastoma cells without significant toxicity.

### F. Sequences of Exemplary Antigen-Binding Proteins

Sequences of exemplary antigen-binding proteins are found in Table 11. Included in Table 11 are orthologs of the proteins, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO molecules listed in Table 11, or a portion thereof. Such polypeptides can have a function of the full-length polypeptide.

### G. Cytokines and Chemokines

Cytokines are small, non-structural proteins of inflammation and immunology. Cytokines affect nearly every biological process; these include embryonic development, disease pathogenesis, non-specific response to infection, specific response to antigen, changes in cognitive functions and progression of the degenerative processes of aging. In addition, cytokines are part of stem cell differentiation, vaccine efficacy and allograft rejection. Multiple biological properties or pleiotropism is the hallmark of a cytokine, and cytokines encompass interferons, the interleukins, chemokines, lymphokines, mesenchymal growth factors, the tumor necrosis factor family and adipokines.

An inflammatory cytokine or proinflammatory cytokine is a type of signaling molecule (a cytokine) that is secreted from immune cells, e.g., helper T cells (Th) and macrophages, and certain other cell types that promote inflammation. They include interleukin-1 (IL-1), IL-12, and IL-18, tumor necrosis factor alpha (TNF-α), interferon gamma (IFNγ), and granulocyte-macrophage colony stimulating factor (GM-CSF) and play an important role in mediating the innate immune response. Inflammatory cytokines are predominantly produced by and involved in the upregulation of inflammatory reactions.

Excessive chronic production of inflammatory cytokines contributes to inflammatory diseases, which have been linked to different diseases, such as atherosclerosis and cancer. Dysregulation has also been linked to depression and other neurological diseases. A balance between proinflammatory and anti-inflammatory cytokines is necessary to maintain health. Aging and exercise also play a role in the amount of inflammation from the release of proinflammatory cytokines.

The major proinflammatory cytokines that are responsible for early responses are IL-1-alpha, IL-1-beta, IL-6, and TNF-α. Other proinflammatory mediators include members of the IL-20 family, IL-33 LIF, IF N-gamma, OS M, CNTF, TGF-beta, GM-CSF, IL-11, IL-12, IL-17, IL-18, IL-8, Rantes, and a variety of other chemokines that chemoattract inflammatory cells. These cytokines either act as endogenous pyrogens (IL-1, IL-6, TNF-α), upregulate the synthesis of secondary mediators and proinflammatory cytokines by both macrophages and mesenchymal cells (including fibroblasts, epithelial and endothelial cells), stimulate the production of acute phase proteins, or attract inflammatory cells.

IL-6 has been shown to play a central role in the neuronal reaction to nerve injury. Suppression of IL-6R by in vivo application of anti-IL-6R antibodies led to reduced regenerative effects. IL-6 is also involved in microglial and astrocytic activation as well as in regulation of neuronal neuropeptides expression. There is evidence that IL-6 contributes to the development of neuropathic pain behavior following a peripheral nerve injury. For example, sciatic cryoneurolysis, a sympathetically-independent model of neuropathic pain involving repeatedly freezing and thawing a section of the sciatic nerve, results in increased IL-6 immunoreactivity in the spinal cord. In addition, intrathecal infusion of IL-6 induces tactile allodynia and thermal hyperalgesia in intact and nerve-injured rats, respectively.

TNF-α, also known as cachectin, is another inflammatory cytokine that plays a well-established, key role in some pain models. TNF acts on several different signaling pathways through two cell surface receptors, TNFR1 and TNFR2 to regulate apoptotic pathways, NF-kB activation of inflammation, and activate stress-activated protein kinases (SAPKs). TNF-α receptors are present in both neurons and glia. TNF-α has been shown to play important roles in both inflammatory and neuropathic hyperalgesia.

Intraplantar injection of complete Freund's adjuvant in adult rats resulted in significant elevation in the levels of TNF-α, IL-1β, and nerve growth factor (NGF) in the inflamed paw. A single injection of anti-TNF-α antiserum before the CFA significantly delayed the onset of the resultant inflammatory hyperalgesia and reduced IL-1β but not NGF levels. Intraplantar injection of TNF-α also produces mechanical and thermal hyperalgesia. It has been found that TNF-α injected into nerves induces Wallerian degeneration and generates the transient display of behaviors and endoneurial pathologies found in experimentally painful nerve injury. TNF binding protein (TNF-BP), an inhibitor of TNF, is a soluble form of a transmembrane TNF-receptor. When TNF-BP is administered systemically, the hyperalgesia normally observed after lipopolysaccharide (LP S) administration is completely eliminated. Intrathecal administration of a combination of TNF-BP and IL-1 antagonist attenuated mechanical allodynia in rats with L5 spinal nerve transection.

Rantes, also known as CCL5, is a chemoattractant for blood monocytes, memory T-helper cells and eosinophils. It causes the release of histamine from basophils and activates eosinophils. It may activate several chemokine receptors including CCR1, CCR3, CCR4 and CCR5. It is one of the major HIV-suppressive factors produced by CD8+ T-cells. Recombinant RANTES protein induces a dose-dependent inhibition of different strains of HIV-1, HIV-2, and simian immunodeficiency virus (SIV). The processed form RANTES (3-68) acts as a natural chemotaxis inhibitor and is a more potent inhibitor of HIV-1-infection. The second processed form RANTES (4-68) exhibits reduced chemotactic and HIV-suppressive activity compared with RANTES (1-68) and RANTES (3-68) and is generated by an unidentified enzyme associated with monocytes and neutrophils.

Rantes may also be an agonist of the G protein-coupled receptor GPR75, stimulating inositol trisphosphate production and calcium mobilization through its activation. Together with GPR75, Rantes may play a role in neuron survival through activation of a downstream signaling pathway involving the PI3, Akt and MAP kinases. Activating GP R75 may also play a role in insulin secretion by islet cells.

Chemokines are a family of small cytokines, or signaling proteins secreted by cells. Their name is derived from their ability to induce directed chemotaxis in nearby responsive cells; they are chemotactic cytokines. In addition to being known for mediating chemotaxis, chemokines are all approximately 8-10 kilodaltons in mass and have four cysteine residues in conserved locations that are key to forming their 3-dimensional shape.

These proteins have historically been known under several other names including the SIS family of cytokines, SIG family of cytokines, SCY family of cytokines, Platelet factor-4 superfamily or intercrines. Some chemokines are considered pro-inflammatory and can be induced during an immune response to recruit cells of the immune system to a site of infection, while others are considered homeostatic and are involved in controlling the migration of cells during normal processes of tissue maintenance or development. Chemokines are found in all vertebrates, some viruses and some bacteria, but none have been described for other invertebrates.

Chemokines represent a family of low molecular weight secreted proteins that primarily function in the activation and migration of leukocytes although some of them also possess a variety of other functions. Chemokines have conserved cysteine residues that allow them to be assigned to four groups: C-C chemokines (monocyte chemoattractant protein or MCP-1, monocyte inflammatory protein or MIP-1α, and MIP-1β), C-X-C chemokines (IL-8 also called growth related oncogene or GRO/KC), C chemokines (lymphotactin), and CXXXC chemokines (fractalkine).

A growth factor is a naturally occurring substance capable of stimulating cellular growth, proliferation, healing, and cellular differentiation. Usually, it is a protein or a steroid hormone. Growth factors are important for regulating a variety of cellular processes.

The antigen-binding proteins of the present disclosure may comprise one or more cytokines, chemokines, and growth factors described above as well as those that are effective in inhibiting tumor metastasis, and wherein the cytokine or growth factor has been shown to have an antiproliferative effect on at least one cell population. Such cytokines, lymphokines, growth factors, or other hematopoietic factors include, but are not limited to: M-CSF, G M-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2 α, cytokine-induced neutrophil chemotactic factor 2 β, β endothelial cell growth factor, endothelin 1, epithelial-derived neutrophil attractant, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof.

### H. VHH and VNAR

Camelids have a special type of antibodies devoid of light chain. These, so-called, heavy chain antibodies (HcAbs) account for up to 50-80% of the circulating antibodies in camels and were also found to be present in the serum of the South American camelids. Camelid HcAbs have a typical IgG Fc region with dedicated isotypes (IgG2 and IgG3) but lack the CH1 constant domain and have a distinctive variable domain (VHH) with structural features that increase its solubility. Other than camelids, HcAbs have not been found in other organisms, with the exception of sharks and other cartilaginous fish (Chondrichthyes), the oldest living beings with an adaptive immune system. In addition to heterotetrameric IgM and IgW, these fishes possess the so-called Ig new antigen receptor (IgNAR). IgNARs are formed by two identical heavy chains composed of five constant domains and a dedicated variable domain (VNAR).

In spite of an evolutionary gap of 425 million years, VHHs and VNARs share some convergent features that differ from those found in conventional variable domains, more notably, changes in conserved amino acids involved in the VH-VL interaction that make them soluble and independently folding domains, non-canonical Cys pairs in CDRs and frameworks (FRs) that increase their stability and diversity, and higher frequency of hypermutation hotspots and longer than average CDR3 that enlarge their recognition repertoire. Formed by fewer CDRs, the antigen-binding sites of VHH and VNAR domains are smaller than those of conventional antibodies, particularly in VNARs that present a deletion of the CDR2 region, and thus are formed by 8 instead of 10 β-strands, making them one of the smallest (12 kDa) antigen-binding domains. The reduced paratope and the frequently extended and flexible CDR3 make VHHs and VNARs particularly capable of binding concave and hidden epitopes (e.g., enzyme active sites, cryptic viral epitopes, etc.) that are not accessible to conventional antibodies. With no distinctive effector functions associated to their constant domains, this unique epitope binding capability has been suggested as the main force that drove the evolution of HcAb. Nevertheless, the reactivity of their antigen-binding site is not limited to hidden targets, and HcAbs reacting with a broad range of structurally diverse epitopes have been described, including flat surfaces in macromolecules and small molecules.

### I. Antigen-Binding Proteins

Provided herein are antigen-binding proteins that have been engineered using the ODIN platform technology, e.g., those comprising the FR2 region, FW2/HV2 region, VH, VL, VHH, or VNAR comprising a heterologous polypeptide. Also provided herein are exemplary antigen-binding proteins that can be incorporated into an ODIN molecule as a heterologous polypeptide.

As described above, the antigen-binding proteins of the present disclosure is not limited to sdAb, but can take any one of many forms of antigen-binding proteins known in the art. In various embodiments, the antigen-binding proteins of the present disclosure take the form of an antibody, or antigen-binding antibody fragment, or an antibody protein product.

In various embodiments of the present disclosure, the antigen-binding protein comprises, consists essentially of, or consists of an antibody or a fragment thereof. As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. Antibody-based antigen-binding proteins comprise the CDRs of the antibody, but not necessarily other regions (e.g., the constant region). The constant region allows the antibody to recruit cells and molecules of the immune system. The variable region is made of the N-terminal regions of each light chain and heavy chain, while the constant region is made of the C-terminal portions of each of the heavy and light chains.

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs within a framework region (designated framework regions 1-4, FR1, FR2, FR3, and FR4.

The term CDR refers to a complementarity determining region (CDR) of which three make up the binding character of a light chain variable region (CDR-L1, CDR-L2 and CDR-L3) and three make up the binding character of a heavy chain variable region (CDR-H1, CDR-H2 and CDR-H3). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions. Despite differing boundaries, each of these systems has some degree of overlap in what constitutes the so called “hypervariable regions” within the variable sequences. CDR definitions according to these systems may therefore differ in length and boundary areas with respect to the adjacent framework region.

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in various embodiments, the antibody is an antibody of isotype IgA, IgD, IgE, IgG, or IgM, including any one of IgG1, IgG2, IgG3 or IgG4. In various aspects, the antibody comprises a constant region comprising one or more amino acid modifications, relative to the naturally-occurring counterpart, in order to improve half-life/stability or to render the antibody more suitable for expression/manufacturability. In various instances, the antibody comprises a constant region wherein the C-terminal Lys residue that is present in the naturally-occurring counterpart is removed or clipped.

The antibody can be a monoclonal antibody. In some embodiments, the antibody comprises a sequence that is substantially similar to a naturally-occurring antibody produced by a mammal, e.g., mouse, rabbit, goat, shark, horse, hamster, human, and the like or by an avian species, e.g., chicken. In this regard, the antibody can be considered as a mammalian antibody, e.g., a mouse antibody, rabbit antibody, goat antibody, shark antibody, horse antibody, hamster antibody, human antibody, and the like, or an avian antibody, e.g., a chicken antibody. In certain aspects, the antigen-binding protein is an antibody, such as a human antibody. In certain aspects, the antigen-binding protein is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species.

The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve selecting amino acid substitutions to make a non-human sequence more similar to a human sequence. Information, including sequence information for human antibody heavy and light chain constant regions is publicly available through the Uniprot database as well as other databases well-known to those in the field of antibody engineering and production. For example, the IgG2 constant region is available from the Uniprot database as Uniprot number P01859, incorporated herein by reference.

An antibody can be cleaved into fragments by enzymes, such as, e.g., papain and pepsin. Papain cleaves an antibody to produce two Fab′ fragments and a single Fc fragment. Pepsin cleaves an antibody to produce a F(ab′)₂ fragment and a pFc′ fragment. In various aspects of the present disclosure, the antigen-binding protein of the present disclosure is an antigen-binding fragment of an antibody (a.k.a., antigen-binding antibody fragment, antigen-binding fragment, antigen-binding portion). In various instances, the antigen-binding antibody fragment is a Fab′ fragment or a F(ab′)₂ fragment.

The architecture of antibodies has been exploited to create a growing range of alternative antibody formats that spans a molecular-weight range of at least about 12-150 kDa and has a valency (n) range from monomeric (n=1), to dimeric (n=2), to trimeric (n=3), to tetrameric (n=4), and potentially higher; such alternative antibody formats are referred to herein as “antibody protein products.” Antibody protein products include those based on the full antibody structure and those that mimic antibody fragments which retain full antigen-binding capacity, e.g., scFvs, Fabs and VHH/VH (discussed below). A soluble, flexible amino acid peptide linker is used to connect the V regions to a scFv (single chain fragment variable) fragment for stabilization of the molecule, or the constant (C) domains are added to the V regions to generate a Fab′ fragment. Both scFv and Fab′ fragments can be easily produced in host cells, e.g., prokaryotic host cells. Other antibody protein products include disulfide-bond stabilized scFv (ds-scFv), single chain Fab′ (scFab′), as well as di- and multimeric antibody formats like dia-, tria- and tetra-bodies, or minibodies (miniAbs) that comprise different formats consisting of scFvs linked to oligomerization domains.

The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. Other antibody protein products include a single chain antibody (SCA); a diabody; a triabody; a tetrabody, and the like.

In various aspects, the antigen-binding protein of the present disclosure comprises, consists essentially of, or consists of any one of these antibody protein products. In various aspects, the antigen-binding protein of the present disclosure comprises, consists essentially of, or consists of any one of an scFv, Fab′, F(ab′)2, VHHNH, Fv fragment, ds-scFv, scFab′, half antibody-scFv, heterodimeric Fab/scFv-Fc, heterodimeric scFv-Fc, heterodimeric IgG (CrossMab), tandem scFv, tandem biparatopic scFv, Fab/scFv-Fc, tandem Fab′, single-chain diabody, dimeric antibody, multimeric antibody (e.g., a diabody, triabody, tetrabody), miniAb, peptibody VHH/VH of camelid heavy chain antibody, sdAb, diabody (single-chain diabody, homodimeric diabody, heterodimeric diabody, tandem diabody (TandAb), diabody that self-dimerizes), a triabody, a tetrabody. An ordinarily skilled artisan would understand that any bispecific antigen-binding protein formats can be used to generate biparatopic antigen-binding protein formats. In some embodiments, the antigen-binding protein is a dual-affinity re-targeting antibody (DART). In some embodiments, the antigen-binding protein is a bispecific T-cell engager (BiTE).

In various aspects, the antigen-binding protein of the present disclosure is linked to an agent. As described below, the agent may be any known in the art, including, but not limited to, chemotherapeutic agents, cytokines and growth factors, cytotoxic agents, detectable agent (e.g., fluorescein), and the like.

The antigen-binding proteins provided herein bind to a target antigen in a non-covalent and reversible manner. In various embodiments, the binding strength of the antigen-binding protein to a target antigen may be described in terms of its affinity, a measure of the strength of interaction between the binding site of the antigen-binding protein and the epitope. In various aspects, the antigen-binding proteins provided herein have high-affinity for the target antigen and thus will bind a greater amount of the target antigen in a shorter period of time than low-affinity antigen-binding proteins. In various aspects, the antigen-binding protein has an equilibrium association constant, K_{A}, which is at least 10⁵ mol⁻¹, at least 10⁶ mol⁻¹, at least 10⁷ mol⁻¹, at least 10⁸ mol⁻¹, at least 109 mol⁻¹, or at least 10¹⁰ mol⁻¹. As understood by the artisan of ordinary skill, K_{A} can be influenced by factors including pH, temperature, and buffer composition.

In various embodiments, the binding strength of the antigen-binding protein to a target antigen may be described in terms of its sensitivity. K_{D} is the equilibrium dissociation constant, a ratio of k_{off}/kₒₙ, between the antigen-binding protein and the target antigen. K_{D} and K_{A} are inversely related. The K_{D} value relates to the concentration of the antigen-binding protein (the amount of antigen-binding protein needed for a particular experiment) and so the lower the K_{D} value (lower concentration) the higher the affinity of the antigen-binding protein. In various aspects, the binding strength of the antigen-binding protein to the target antigen may be described in terms of K_{D}. In various aspects, the K_{D} of the antigen-binding proteins provided herein is about 10⁻¹, about 10⁻², about 10⁻³, about 10⁻⁴, about 10⁻⁵, about 10⁻⁶, or less. In various aspects, the K_{D} of the antigen-binding proteins provided herein is micromolar, nanomolar, picomolar or femtomolar. In various aspects, the K_{D} of the antigen-binding proteins provided herein is within a range of about 10⁻⁴ to 10⁻⁶ or 10⁻⁷ to 10⁻⁹ or 10⁻¹⁰ to 10⁻¹² or 10⁻¹³ to 10⁻¹⁵. In various aspects, the K_{D} of the antigen-binding proteins provided herein is within a range of about 1.0×10⁻¹² M to about 1.0×10⁻⁸ M. In various aspects, the K_{D} of the antigen-binding proteins is within a range of about 1.0×10⁻¹¹ M to about 1.0×10⁻⁹ M.

In various aspects, the affinity of the antigen-binding proteins is measured or ranked using a flow cytometry- or Fluorescence-Activated Cell Sorting (FACS)-based assay. Flow cytometry-based binding assays are known in the art. In various aspects, the affinity of the antigen-binding proteins is measured or ranked using a competition assay.

Avidity gives a measure of the overall strength of an antigen-binding protein-antigen complex. It is dependent on three major parameters: affinity of the antigen-binding protein for the epitope, valency of both the antigen-binding protein and the target antigen, and structural arrangement of the parts that interact. The greater an antigen-binding protein's valency (number of antigen binding sites), the greater the amount of antigen it can bind. In various aspects, the antigen-binding proteins have a strong avidity for the target antigen. In various aspects, the antigen-binding proteins are multivalent. In various aspects, the antigen-binding proteins are bivalent. In various instances, the antigen antigen-binding proteins are monovalent.

In some embodiments, the engineered antigen-binding protein comprises an antibody, Fv, F(ab′)2, Fab′, dsFv, scFv, sc(Fv)2, half antibody-scFv, tandem scFv, tandem biparatopic scFv, Fab/scFv-Fc, tandem Fab′, single-chain diabody, tandem diabody (TandAb), Fab/scFv-Fc, heterodimeric Fab/scFv-Fc, heterodimeric scFv-Fc, heterodimeric IgG (CrossMab), DART, and diabody.

In certain embodiments, the engineered antigen-binding protein comprises an immunoglobulin heavy chain constant domain selected from the group consisting of IgG, IgG1, IgG2, IgG2A, IgG2B, IgG3, IgG4, IgA, IgM, IgD, and IgE constant domains.

In some embodiments, the engineered antigen-binding protein comprises an Fc domain. In some embodiments, the Fc domain is a functional or wild-type Fc domain that can bind to one or more Fc receptors. In some embodiments, the Fc domain may be nonfunctional, e.g., comprise a mutation, deletion, substitution, addition of one or more critical amino acids such that Fc domain (while structurally present) can no longer bind to one or more Fc receptors. In other embodiments, the engineered antigen-binding protein does not comprise (a) an Fc domain, or (b) the CH2 domain and/or CH3 domain of the constant region of an antibody. Accordingly, in some embodiments, the engineered antigen-binding protein does not bind to one or more Fc receptors, irrespective of whether the engineered antigen-binding protein comprises the Fc domain.

In some embodiments, the engineered antigen-binding protein comprises at least two different VH domains and at least two different V_{L} domains.

In certain aspects, provided herein is an isolated nucleic acid molecule that encodes the engineered antigen-binding protein of the present disclosure. Also provided herein is a vector comprising such isolated nucleic acid. Further provided herein is a host cell which comprises the said isolated nucleic acid, comprises the said vector, or expresses the engineered antigen-binding protein of the present disclosure.

In certain aspects, provided herein is a pharmaceutical composition comprising the engineered antigen-binding protein of the present disclosure, an isolated nucleic acid that encodes said engineered antigen-binding protein, a vector comprising said isolated nucleic acid, or a host cell comprising the said isolated nucleic acid, comprises the said vector, or expresses the engineered antigen-binding protein of the present disclosure.

In certain aspects, provided herein is a kit comprising at least one engineered antigen-binding protein of the present disclosure.

### J. Antibody Engineering to Improve Pharmacokinetics (PK)

In other embodiments, an antigen-binding protein can be engineered to increase or improve its pharmacokinetic (PK) properties (e.g., half-life). Numerous properties of an antigen-binding protein can influence pharmacokinetics including, but not limited to, molecular size, folding stability, solubility, target interaction, neonatal Fc binding capacity, and isoelectric point (pI). Modifications to the antigen-binding protein include, but are not limited to, antigen-binding domain conjugation to one or more carrier proteins, PEGylation, acylation (e.g., by conjugation to a fatty acid molecule), polysialylation, or glycosylation. Amino acid sequence modifications can be used to improve or optimize the PK properties of the protein, and conjugation to large, slowly metabolized macromolecules can also modify the PK properties of the protein. Macromolecules that can be conjugated to the antigen protein include, but are not limited to, proteins (e.g., albumin or albumin-binding protein; such can also be expressed as a fusion protein), polysaccharides (e.g., sepharose, agarose, cellulose, or cellulose beads), polymeric amino acids (polyglutamic acid or polylysine), amino acid copolymers, inactivated virus particles, inactivated bacterial toxins (e.g., leukotoxin or diphtheria, tetanus, or cholera toxins or molecules), inactivated bacteria, dendritic cells, thyroglobulin, polyamino acids (e.g., poly(D-lysine:D-glutamic acid)), VP 6 polypeptides of rotaviruses, influenza virus hemaglutinin, influenza virus nucleoprotein, Keyhole Limpet Hemocyanin (KLH), and hepatitis B virus core protein and surface antigen. Additional PK modulators known in the art include lipophiles, bile acids, steroids, phospholipid analogues, and vitamins, examples of which include, but are not limited to, cholesterol, fatty acids, cholic acid, lithocholic acid, dialkylglycerides, diacylglyceride, phospholipids, sphingolipids, naproxen, ibuprofen, vitamin E, and biotin. Methods for producing modified antigen-binding proteins as described herein are known in the art. Macromolecules can be conjugated to the antigen-binding protein via a site-specific conjugation. PEGylation via site-specific conjugation is known in the art. Additional methods of extending the PK are also described.

In some embodiments, the antigen-binding protein is fused or otherwise linked to a conventional fragment crystallizable region (Fc Region) or a fragment thereof. For example, the Fc region can be an IgGI, IgG2, IgG3, or IgG4 Fc region. In some embodiments, mutations in the Fc region of the antigen-binding protein can be engineered to modulate its interaction with the neonatal Fc receptor (FcRn), which is involved in receptor-mediated internalization and recycling of IgG occur via FcRn, thereby improving its pharmacokinetic properties. In some embodiments, the antigen-binding protein is fused or otherwise linked to an albumin-binding protein.

In addition to a conventional Fc region or a fragment thereof, there are engineered FcRN binding peptides, when fused to a protein, that significantly enhance the half-life of the protein in primates. Such peptides include small linear and cyclic FcRn binding peptides (collectively called FcRnBPs) that can be fused to a combination of the N- and C-termini of a protein, e.g., Fab, to improve the pharmacokinetics of the protein. Such peptides include those having an exemplary amino acid sequence of QRFCTGHFGGLYPCNG; QRFCTGHFGGLHPCNG; QRFVTGHFGGLYPANG; or QRFVTGHFGGLHPANG.

In some embodiments, the macromolecule is directly conjugated to the antigen-binding protein. In some embodiments, the macromolecule is fused to the antigen-binding peptide via a linker.

Modified antigen-binding proteins as described herein can have improved or optimized pharmacokinetic (PK) properties, for example, a plasma half-life in a human subject of greater than 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 10 days, 14 days, or 30 days.

Methods of testing the antigen-binding protein for the ability to bind to the epitope(s) regardless of how the antigen-binding proteins are produced are known in the art and include any binding assay, such as, for example, radioimmunoassay (RIA), ELISA, Western blot, immunoprecipitation, SPR, and competitive inhibition assays, for example.

### K. Sequence Identity and Homology

Functionally-conservative variants are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A function-conservative variant also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

The percent identity between two sequences is a function of the number of identical positions shared by the sequences (i.e., % identity=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package using a NWSgapdna. CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:1117 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444 453 (1970)) algorithm which has been incorporated into the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6.

The nucleic acid and protein sequences of the present disclosure can further be used as a “query sequence” to perform a search against public databases to, for example, identify related sequences. Such searches can be performed using the NBLAST and XBLAST programs (version 2.0) of BLAST nucleotide searches can be performed with the NBLAST program, score=100, wordlength=12 to obtain nucleotide sequences homologous to the nucleic acid molecules of the present disclosure. BLAST protein searches can be performed with the XBLAST program, score=50, wordlength=3 to obtain amino acid sequences homologous to the protein molecules of the present disclosure. To obtain gapped alignments for comparison purposes, Gapped BLAST can also be utilized. When utilizing BLAST and Gapped BLAST programs, the default parameters of the respective programs (e.g., XBLAST and NBLAST can be used.

### L. Engineered Nucleic Acids and Vectors

Further provided herein are isolated nucleic acid molecules that encode the FR2 region or FW2/HV2 region, the VH domain, the VL domain, the VHH domain, or the VNAR domain of the present disclosure, each of which comprises a heterologous polypeptide. Also provided herein are the nucleic acid molecules that encode the antigen-binding proteins or the chimeric antigen receptors of the present disclosure.

In certain aspects, provided herein is a vector comprising the above nucleic acid molecule. Typically, the engineered nucleic acid is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence. Thus, a further object of the disclosure relates to a vector comprising a nucleic acid of the present disclosure.

Such vectors may comprise regulatory elements, such as a promoter, enhancer, terminator and the like, to cause or direct expression of said polypeptide upon administration to a subject. Examples of promoters and enhancers used in the expression vector for animal cells include early promoter and enhancer of SV40, LTR promoter and enhancer of Moloney mouse leukemia virus, promoter and enhancer of immunoglobulin H chain and the like.

Any expression vector for animal cells can be used. Examples of suitable vectors include pAGE107, pAGE103, pHSG274, pKCR, pSG1 beta d2-4 and the like. Other representative examples of plasmids include replicating plasmids comprising an origin of replication, or integrative plasmids, such as for instance pUC, pcDNA, pBR, and the like. Representative examples of viral vectors include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc.

Accordingly, the nucleic acids of the present disclosure in some embodiments are incorporated into a vector. In this regard, the present disclosure provides vectors comprising any of the presently disclosed nucleic acids. In various aspects, the vector is a recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the present disclosure are not naturally-occurring as a whole. However, parts of the vectors can be naturally-occurring. The presently disclosed vectors can comprise any type of nucleotides, including, but not limited to DNA and RNA, which can be single-stranded or double-stranded, synthesized or obtained in part from natural sources, and which can contain natural, non-natural or altered nucleotides. The vectors can comprise naturally-occurring or non-naturally-occurring internucleotide linkages, or both types of linkages. In some embodiments, the altered nucleotides or non-naturally occurring internucleotide linkages do not hinder the transcription or replication of the vector.

The vector of the present disclosure can be any suitable vector, and can be used to transduce, transform or transfect any suitable host. Suitable vectors include those designed for propagation and expansion or for expression or both, such as plasmids and viruses. The vector can be a plasmid-based expression vector. In various aspects, the vector is selected from the group consisting of the pUC series (Fermentas Life Sciences), the pBluescript series (Stratagene, LaJ olla, CA), the pET series (Novagen, Madison, WI), the pGEX series (Pharmacia Biotech, Uppsala, Sweden), and the pEX series (Clontech, Palo Alto, CA). Bacteriophage vectors, such as AGTIO, λGTI1, λZapII (Stratagene), λEMBL4, and λNMI 149, also can be used. Examples of plant expression vectors include pBIOI, pBI101.2, pBI101.3, pBI121 and pBIN19 (Clontech). Examples of animal expression vectors include pEUK-CI, pMAM and pMAMneo. In some aspects, the vector is a viral vector, e.g., a retroviral vector. In various aspects, the vector is an adenovirus vector, an adeno-associated virus (AAV) vector, a Herpes Simplex Virus (HSV) vector, a Vesicular stomatitis virus (VSV) vector, vaccinia virus vector, or lentivirus vector. In various aspects, the vector is a baculovirus vector which infects arthropods, e.g., insects. In various aspects, the baculovirus vector is an Autographacalifornica multiple nuclear virus (AcMNPV) or a Bombyxmorinuclear polyhedrosis (BmNPV).

The vectors of the present disclosure can be prepared using standard recombinant DNA techniques. Constructs of expression vectors, which are circular or linear, can be prepared to contain a replication system functional in a prokaryotic or eukaryotic host cell. Replication systems can be derived, e.g., from ColEI, 2μ plasmid, λ, SV40, bovine papilloma virus, and the like.

In some aspects, the vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the presently disclosed expression vectors include, for instance, neomycin/G418 resistance genes, hygromycin resistance genes, histidinol resistance genes, tetracycline resistance genes, and ampicillin resistance genes.

The vector can comprise a native or normative promoter operably linked to the nucleotide sequence encoding the polypeptide (including functional portions and functional variants thereof), or to the nucleotide sequence which is complementary to or which hybridizes to the nucleotide sequence encoding the polypeptide. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

In another aspect, the present disclosure provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present disclosure can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. Preferably, the cDNA library comprises at least 80% full-length sequences, preferably, at least 85% or 90% full-length sequences, and, preferably, at least 95% full-length sequences. The cDNA libraries can be normalized to increase the representation of rare sequences.

Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides of this technology will encode at least a portion of an antibody encoded by the polynucleotides described herein. The polynucleotides of the present disclosure embrace nucleic acid sequences that can be employed for selective hybridization to a polynucleotide encoding an antibody of the present disclosure.

In other embodiments, provided herein is a virus comprising the FR2 region or FW2/HV2 region, the VH domain, the VL domain, the VHH domain, the VNAR domain, or the antigen-binding protein or the chimeric antigen receptor comprising same, of the present disclosure, each of which comprises a heterologous polypeptide. Also provided herein is a cell comprising the virus of the present disclosure.

In some embodiments, the virus is a bacteriophage (e.g., for phage display), a vaccinia (e.g., for vaccinia display), or an AAV.

A virus or viral vector comprising the FR2 region or FW2/HV2 region, the VH domain, the VL domain, the VHH domain, the VNAR domain, or the antigen-binding protein or the chimeric antigen receptor comprising same, of the present disclosure, each of which comprises a heterologous polypeptide; or the nucleic acid encoding same is useful in phage display or vaccinia display. For example, the heterologous antigen-binding protein (e.g., UL-CDR3 inserted in the FR2 region, FW2/HV2 region, the VH domain, the VL domain, the VHH domain, or the VNAR domain, etc.) of the present disclosure can be engineered to introduce randomized sequences. The heterologous antigen-binding protein comprising said randomized sequences can be displayed on phage, virus, or yeast to facilitate screening of the randomized sequences. Such techniques, e.g., phage display, vaccinia display, or yeast display are well known in the art.

Furthermore, a virus such as AAV (e.g., a virus used for gene therapy) comprising the FR2 region or FW2/HV2 region, the VH domain, the VL domain, the VHH domain, the VNAR domain, or the antigen-binding protein or the chimeric antigen receptor comprising same, of the present disclosure, each of which comprises a heterologous polypeptide; or the nucleic acid encoding same is useful in treating a subject, e.g., a subject afflicted with a disease described herein. Gene therapy (e.g., AAV-mediated) can introduce a stable source of antigen-binding protein of the present disclosure for a chronic condition or a disease that requires continued supply of said antigen-binding protein. Such gene therapy has been combined with CAR therapies for cancer treatment.

In other aspects, provided herein is a cell comprising the FR2 region or FW2/HV2 region, the VH domain, the VL domain, the VHH domain, or the VNAR domain of the present disclosure, each of which comprises a heterologous polypeptide. Also provided herein is a cell comprising the antigen-binding protein or the chimeric antigen receptor of the present disclosure. In addition, provided herein is a cell comprising the nucleic acid or the vector of the present disclosure.

In some embodiments, the cell is a prokaryotic cell or a eukaryotic cell.

In some embodiments, the eukaryotic cell is a mammalian cell or a fungus (e.g., yeast, e.g., Pichia pastoris). In some embodiments, the prokaryotic cell is a bacterium. In some embodiments, the cell is a human cell. In some such embodiments, the cell may be a T cell, an NK cell, or a macrophage (e.g., CAR-T, CAR-NK, CAR-M).

A further object of the present disclosure relates to a cell which has been transfected, infected or transformed by a nucleic acid and/or a vector according to the present disclosure. The term “transformation” means the introduction of a “foreign” (i.e., extrinsic or extracellular) gene, DNA or RNA sequence to a cell, so that the cell will express the introduced gene or sequence to produce a desired substance, typically a protein or enzyme coded by the introduced gene or sequence. A cell that receives and expresses introduced DNA or RNA has been “transformed.”

The nucleic acids of the present disclosure may be used to produce a recombinant polypeptide of the present disclosure in a suitable expression system. The term “expression system” means a cell and compatible vector under suitable conditions, e.g., for the expression of a protein coded for by foreign DNA carried by the vector and introduced to the cell.

Common expression systems include E. coli cells and plasmid vectors, insect cells and Baculovirus vectors, and mammalian cells and vectors. Other examples of cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

The present disclosure also relates to a method of producing a recombinant cell expressing an antibody or a polypeptide of the present disclosure according to the present disclosure, said method comprising the steps consisting of (i) introducing in vitro or ex vivo a recombinant nucleic acid or a vector as described herein into a competent cell, (ii) culturing in vitro or ex vivo the recombinant cell obtained and (iii), optionally, selecting the cells which express and/or secrete said antibody or polypeptide. Such recombinant cells can be used for the production of antibodies and polypeptides of the present disclosure.

Any type of cell that can contain the presently disclosed vector and is capable of producing an expression product encoded by the nucleic acid (e.g., mRNA, protein). The cell in some aspects is an adherent cell or a suspended cell, i.e., a cell that grows in suspension. The cell in various aspects is a cultured cell or a primary cell, i.e., isolated directly from an organism, e.g., a human. The cell can be of any cell type, can originate from any type of tissue, and can be of any developmental stage.

In certain aspects, the antigen-binding protein is a glycosylated protein, and the cell is a glycosylation-competent cell. In various aspects, the glycosylation-competent cell is a eukaryotic cell, including, but not limited to, a yeast cell, filamentous fungi cell, protozoa cell, algae cell, insect cell, or mammalian cell. Such cells are described in the art. In various aspects, the eukaryotic cells are mammalian cells.

In various aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NS0, GS-NS0, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinomic human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J 558L, or baby hamster kidney (BHK) cells.

For purposes of amplifying or replicating the vector, the cell is in some aspects is a prokaryotic cell, e.g., a bacterial cell. Also provided is a population of cells comprising at least one cell described herein. The population of cells in some aspects is a heterogeneous population comprising the cell comprising vectors described, in addition to at least one other cell, which does not comprise any of the vectors. Alternatively, in some aspects, the population of cells is a substantially homogeneous population, in which the population comprises mainly cells (e.g., consisting essentially of) comprising the vector. The population in some aspects is a clonal population of cells, in which all cells of the population are clones of a single cell comprising a vector, such that all cells of the population comprise the vector. In various embodiments of the present disclosure, the population of cells is a clonal population comprising cells comprising a vector as described herein.

In certain aspects the cell is a human cell that is autologous or allogeneic to the subject. In some embodiments, a nucleic acid of the present disclosure is transduced via a viral vector or transformed in other suitable methods (e.g., electroporation, etc.). Such cells are transferred (e.g., grafted, implanted, etc.) to the subject for a prolonged treatment of the disease or condition, e.g., cancer.

### M. Manufacturing Methods

Also provided herein are methods of producing constructs with the FR2 region or FW2/HV2 region, the VH domain, the VL domain, the VHH domain, the VNAR domain, or the antigen-binding protein or the chimeric antigen receptor comprising same, of the present disclosure, each of which comprises a heterologous polypeptide.

In various embodiments, the method comprises culturing a cell comprising a nucleic acid comprising a nucleotide sequence encoding the antigen-binding protein as described herein in a cell culture medium (e.g., under conditions suitable to allow expression), and harvesting/recovering the expressed FR2 region, FW/HV2 region, VHH domain, VL domain, VHH domain, VNAR domain, antigen-binding protein, or chimeric antigen receptor from the cell culture medium. The cell can be any of the cells described herein. In some embodiments, the cell is a prokaryotic cell or a eukaryotic cell. In some embodiments, the eukaryotic cell is a mammalian cell or a fungus (e.g., yeast, e.g., Pichia pastoris). In some embodiments, the prokaryotic cell is a bacterium.

In various aspects, the cell is selected from the group consisting of: CHO cells, NS0 cells, COS cells, VERO cells, and BHK cells. In various aspects, the step of culturing a cell comprises culturing the cell in a growth medium to support the growth and expansion of the cell. In various aspects, the growth medium increases cell density, culture viability and productivity in a timely manner. In various aspects, the growth medium comprises amino acids, vitamins, inorganic salts, glucose, and serum as a source of growth factors, hormones, and attachment factors. In various aspects, the growth medium is a fully chemically defined media consisting of amino acids, vitamins, trace elements, inorganic salts, lipids and insulin or insulin-like growth factors. In addition to nutrients, the growth medium also helps maintain pH and osmolality. Several types of growth media are commercially available and are described in the art.

In various aspects, the method comprises culturing the cell in a feed medium. In various aspects, the method comprises culturing in a feed medium in a fed-batch mode. Methods of recombinant protein production are known in the art.

The method of making an antigen-binding protein can comprise one or more steps for purifying the protein from a cell culture or the supernatant thereof and preferably recovering the purified protein. In various aspects, the method comprises one or more chromatography steps, e.g., affinity chromatography (e.g., protein A affinity chromatography, nickel resin for Histidine (His) tags), ion exchange chromatography, hydrophobic interaction chromatography. In various aspects, the method comprises purifying the protein using a Protein A affinity chromatography resin if the antigen-binding protein comprises an Fc domain.

In various embodiments, the method further comprises steps for formulating the purified protein, etc., thereby obtaining a formulation comprising the purified protein.

In various aspects, the antigen-binding protein linked to a polypeptide and the antigen-binding protein is part of a fusion protein. Thus, the present disclosure further provides methods of producing a fusion protein comprising an antigen-binding protein. In various embodiments, the method comprises culturing a cell comprising a nucleic acid comprising a nucleotide sequence encoding the fusion protein as described herein in a cell culture medium and harvesting the fusion protein from the cell culture medium.

Accordingly, the engineered antigen-binding protein of the present disclosure may be produced by any technique known in the art, such as, without limitation, any chemical, biological, genetic or enzymatic technique, either alone or in combination.

Knowing the amino acid sequence of the desired sequence, one skilled in the art can readily produce said antibodies or polypeptides, by standard techniques for production of polypeptides. For instance, they can be synthesized using well-known solid phase methods, preferably using a commercially available peptide synthesis apparatus and following the manufacturer's instructions. Alternatively, antibodies and other polypeptides of the present disclosure can be synthesized by recombinant DNA techniques as is well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

In particular, the present disclosure further relates to a method of producing an antigen-binding protein or a fragment thereof, which method comprises the steps consisting of: (i) culturing a transformed cell according to the present disclosure under conditions suitable to allow expression of said antigen-binding protein or a fragment thereof; and (ii) recovering the expressed antigen-binding protein or a fragment thereof.

An engineered antigen-binding protein of the present disclosure are suitably separated from the culture medium by conventional immunoglobulin purification procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography and lectin chromatography. High performance liquid chromatography (“HPLC”) can also be employed for purification.

Chimeric antibodies (e.g., mouse-human chimeras or non-rodent-human chimeras) of the present disclosure can be produced by obtaining nucleic sequences encoding V_{L} and V_{H} domains as previously described, constructing a human chimeric antibody expression vector by inserting them into an expression vector for animal cell having genes encoding human antibody CH and human antibody CL, and expressing the coding sequence by introducing the expression vector into an animal cell. The CH domain of a human chimeric antibody can be any region which belongs to human immunoglobulin, such as the IgG class or a subclass thereof, such as IgG1, IgG2, IgG3 and IgG4.

Similarly, the CL of a human chimeric antibody can be any region which belongs to Ig, such as the kappa class or lambda class. The chimeric and humanized monoclonal antibodies, comprising both human and non-human portions, which can be made using standard recombinant DNA techniques, are within the scope of the present disclosure. Such chimeric and humanized monoclonal antibodies can be produced by recombinant DNA techniques known in the art.

In addition, humanized antibodies can be made according to standard protocols that are known in the art. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art. Humanized antibodies of the present disclosure can be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell.

The humanized antibody expression vector may be either of a type in which a gene encoding an antibody heavy chain and a gene encoding an antibody light chain exists on separate vectors or of a type in which both genes exist on the same vector (tandem type).

Methods for producing humanized antibodies based on conventional recombinant DNA and gene transfection techniques are well-known in the art For example, antibodies can be humanized using a variety of techniques known in the art including, for example, CDR-grafting, veneering or resurfacing and chain shuffling. The general recombinant DNA technology for preparation of such antibodies is also known.

In addition, methods for producing antibody fragments are well-known. For example, Fab fragments of the present disclosure can be obtained by treating an antibody which specifically reacts with a ganglioside with a protease such as papain. Also, Fabs can be produced by inserting DNA encoding Fabs of the antibody into a vector for prokaryotic expression system, or for eukaryotic expression system, and introducing the vector into a prokaryote or eukaryote (as appropriate) to express the Fabs.

Similarly, F(ab′)2 fragments of the present disclosure can be obtained treating an antibody which specifically reacts with a ganglioside with a protease, pepsin. Also, the F(ab′)2 fragment can be produced by binding Fab′ described below via a thioether bond or a disulfide bond.

Fab′ fragments of the present disclosure can be obtained treating F(ab′)2 which specifically reacts with a ganglioside with a reducing agent, dithiothreitol. Also, the Fab′ fragments can be produced by inserting DNA encoding a Fab′ fragment of the antibody into an expression vector for prokaryote, or an expression vector for eukaryote, and introducing the vector into a prokaryote or eukaryote (as appropriate) to perform its expression.

In addition, scFvs of the present disclosure can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well-known technology called CDR grafting may be used, which involves selecting the complementary determining regions (CDRs) from a donor scFv fragment and grafting them onto a human scFv fragment framework of known three-dimensional structure. The engineered antigen-binding protein of the present disclosure can be produced using a variety of methods well known in the art, including de novo protein synthesis and recombinant expression of nucleic acids encoding the binding proteins. The desired nucleic acid sequences can be produced by recombinant methods or by solid-phase DNA synthesis.

### N. Modification of Antigen-binding Proteins

Amino acid sequence modification(s) of the antigen-binding proteins described herein are contemplated. For example, it may be desirable to improve the binding affinity and/or other biological properties. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce binding activity and can be corrected by replacing the amino acids with amino acid residues of the original antibody derived from a non-human animal.

Modifications and changes may be made in the structure of the antibodies of the present disclosure, and in the DNA sequences encoding them, and still obtain a functional molecule that encodes an antibody and polypeptide with desirable characteristics. For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences of the present disclosure, or corresponding DNA sequences that encode said polypeptides, without appreciable loss of their biological activity.

In one embodiment, amino acid changes may be achieved by changing codons in the DNA sequence to encode conservative substitutions based on conservation of the genetic code. Specifically, there is a known and definite correspondence between the amino acid sequence of a particular protein and the nucleotide sequences that can code for the protein, as defined by the genetic code. Likewise, there is a known and definite correspondence between the nucleotide sequence of a particular nucleic acid and the amino acid sequence encoded by that nucleic acid, as defined by the genetic code.

As described above, an important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In making the changes in the amino sequences of polypeptide, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (<RTI 3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take on various qualities of the foregoing characteristics into consideration are well-known to those of skill in the art and include: arginine and lysine; glutamate and aspartate; serine and threonine; glutamine and asparagine; and valine, leucine and isoleucine.

Another type of amino acid modification of the antigen-binding protein of the present disclosure may be useful for altering the original glycosylation pattern of the antibody to, for example, increase stability. The term “altering” means deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine.

Similarly, removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetyl galactosamine), while leaving the antibody intact. Enzymatic cleavage of carbohydrate moieties on antibodies can be achieved by the use of a variety of endo- and exo-glycosidases.

Other modifications can involve the formation of immunoconjugates. For example, in one type of covalent modification, antibodies or proteins are covalently linked to one of a variety of non-proteinaceous polymers, e.g., polyethylene glycol, polypropylene glycol, or polyoxyalkylenes.

Conjugation of antigen-binding protein of the present disclosure with heterologous agents can be made using a variety of bifunctional protein coupling agents including but not limited to N-succinimidyl (2-pyridyldithio) propionate (S PDP), succinimidyl (N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6 diisocyanate), and bis-active fluorine compounds (such as 1,5-difluoro-2,4-dinitrobenzene). For example, carbon labeled 1-isothiocyanatobenzyl methyldiethylene triaminepentaacetic acid (MX-DTPA) is an exemplary chelating agent for conjugation of radionucleotide to the antibody.

In another aspect, the present disclosure features an antigen-binding protein conjugated to a moiety that allows detection in vivo or in vitro. Conjugated antigen-binding protein can be used to monitor its presence in blood or tissues as part of a clinical testing procedure. Examples of detectable moieties include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin (P E); an example of a luminescent material includes luminol; examples of bioluminescent materials include luciferase, luciferin, and aequorin, and examples of suitable radioactive material include ¹²⁵I, ¹³¹I, ³⁵S, or ³H. As used herein, the term “labeled”, with regard to the antibody, is intended to encompass direct labeling of the antibody by coupling (i.e., physically linking) a detectable substance, such as a radioactive agent or a fluorophore (e.g. fluorescein isothiocyanate (FITC) or phycoerythrin (PE) or indocyanine (Cy5)) to the antibody, as well as indirect labeling of the antibody by reactivity with a detectable substance. For example, an antibody may be labeled with a nucleic acid sequence that may be amplified and detected, or an antisense oligonucleotide to reduce expression of a particular gene, such that expression can then be detected and measured. Techniques for conjugating such therapeutic moiety to an antigen-binding protein (e.g., antibody or fragments thereof) are well-known.

### O. Conjugates

The present disclosure also provides a conjugate comprising an FR2 region, an FW2/HV2 region, a VH domain, a VL domain, a VHH domain, a VNAR domain, or an antigen-binding protein. In some embodiments, the conjugate comprises a polyethylene glycol (PEG), a chemotherapeutic agent, and/or a cytotoxic agent.

Specifically, antigen-binding proteins attached, linked or conjugated to a second moiety (e.g., a heterologous moiety, a conjugate moiety). Accordingly, the present disclosure provides a conjugate comprising an antigen-binding protein and a heterologous moiety. As used herein, the term “heterologous moiety” is synonymous with “conjugate moiety” and refers to any molecule (chemical or biochemical, naturally-occurring or non-coded) which is different from the antigen-binding proteins of the present disclosure. Various heterologous moieties include, but are not limited to, a polymer, a carbohydrate, a lipid, a nucleic acid, an oligonucleotide, a DNA or RNA, an amino acid, peptide, polypeptide, protein, therapeutic agent, (e.g., a cytotoxic agent, cytokine), or a diagnostic agent.

In some embodiments, the conjugation produces heterogeneous population of conjugates. In other embodiments, the conjugation (e.g., site-specific conjugation) produces a substantially homogeneous population of conjugates. Methods of heterogenous conjugation and site-specific conjugation are well known in the art.

In some embodiments, the heterologous moiety is conjugated to the antigen-binding protein (e.g., antibody) in a site-specific manner. Various site-specific conjugation methods are known in the art, e.g., thiomab or TDC or conjugation at an unpaired cysteine residue; thiol bridge linker; conjugation at glutamine using a transglutaminase; conjugation at engineered unnatural amino acid residues; selenocysteine conjugation; glycan-mediated conjugation; conjugation at galactose or GalNAc analogues; via glycan engineering; via a short peptide tag, such as engineering a glutamine tag or sortase; and via an aldehyde tag, for example.

In some embodiments, the heterologous moiety is a polymer. The polymer can be branched or unbranched. The polymer can be of any molecular weight. The polymer in some embodiments has an average molecular weight of between about 2 kDa to about 100 kDa (the term “about” indicating that in preparations of a water-soluble polymer, some molecules will weigh more, some less, than the stated molecular weight). The average molecular weight of the polymer is in some aspect between about 5 kDa and about 50 kDa, between about 12 kDa to about 40 kDa or between about 20 kDa to about 35 kDa.

In some embodiments, the polymer is modified to have a single reactive group, such as an active ester for acylation or an aldehyde for alkylation, so that the degree of polymerization can be controlled. The polymer in some embodiments is water soluble so that the protein to which it is attached does not precipitate in an aqueous environment, such as a physiological environment. In some embodiments, when, for example, the composition is used for therapeutic use, the polymer is pharmaceutically acceptable. Additionally, in some aspects, the polymer is a mixture of polymers, e.g., a co-polymer, a block co-polymer.

In some embodiments, the polymer is selected from the group consisting of: polyamides, polycarbonates, polyalkylenes and derivatives thereof including, polyalkylene glycols, polyalkylene oxides, polyalkylene terepthalates, polymers of acrylic and methacrylic esters, including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), polyvinyl polymers including polyvinyl alcohols, polyvinyl ethers, polyvinyl esters, polyvinyl halides, poly(vinyl acetate), and polyvinylpyrrolidone, polyglycolides, polysiloxanes, polyurethanes and copolymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, polypropylene, polyethylenes including poly(ethylene glycol), poly(ethylene oxide), and poly(ethylene terephthalate), and polystyrene.

A particularly preferred water-soluble polymer for use herein is polyethylene glycol (PEG). As used herein, polyethylene glycol is meant to encompass any of the forms of PEG that can be used to derivatize other proteins, such as mono-(C1-C10) alkoxy- or aryloxy-polyethylene glycol. PEG is a linear or branched neutral polyether, available in a broad range of molecular weights, and is soluble in water and most organic solvents.

In some embodiments, the heterologous moiety is a carbohydrate. In some embodiments, the carbohydrate is a monosaccharide (e.g., glucose, galactose, fructose), a disaccharide (e.g., sucrose, lactose, maltose), an oligosaccharide (e.g., raffinose, stachyose), a polysaccharide (a starch, amylase, amylopectin, cellulose, chitin, callose, laminarin, xylan, mannan, fucoidan, galactomannan.

In some embodiments, the heterologous moiety is a lipid. The lipid, in some embodiments, is a fatty acid, eicosanoid, prostaglandin, leukotriene, thromboxane, N-acyl ethanolamine), glycerolipid (e.g., mono-, di-, tri-substituted glycerols), glycerophospholipid (e.g., phosphatidylcholine, phosphatidylinositol, phosphatidylethanolamine, phosphatidylserine), sphingolipid (e.g., sphingosine, ceramide), sterol lipid (e.g., steroid, cholesterol), prenol lipid, saccharolipid, or a polyketide, oil, wax, cholesterol, sterol, fat-soluble vitamin, monoglyceride, diglyceride, triglyceride, a phospholipid.

In some embodiments, the heterologous moiety is a therapeutic agent. The therapeutic agent can be any of those known in the art. Examples of therapeutic agents that are contemplated herein include, but are not limited to, natural enzymes, proteins derived from natural sources, recombinant proteins, natural peptides, synthetic peptides, cyclic peptides, antibodies, receptor agonists, cytotoxic agents, immunoglobins, beta-adrenergic blocking agents, calcium channel blockers, coronary vasodilators, cardiac glycosides, antiarrhythmics, cardiac sympathomemetics, angiotensin converting enzyme (ACE) inhibitors, diuretics, inotropes, cholesterol and triglyceride reducers, bile acid sequestrants, fibrates, 3-hydroxy-3-methylgluteryl (HMG)-CoA reductase inhibitors, niacin derivatives, antiadrenergic agents, alpha-adrenergic blocking agents, centrally acting antiadrenergic agents, vasodilators, potassium-sparing agents, thiazides and related agents, angiotensin II receptor antagonists, peripheral vasodilators, antiandrogens, estrogens, antibiotics, retinoids, insulins and analogs, alpha-glucosidase inhibitors, biguanides, meglitinides, sulfonylureas, thizaolidinediones, androgens, progestogens, bone metabolism regulators, anterior pituitary hormones, hypothalamic hormones, posterior pituitary hormones, gonadotropins, gonadotropin-releasing hormone antagonists, ovulation stimulants, selective estrogen receptor modulators, antithyroid agents, thyroid hormones, bulk forming agents, laxatives, antiperistaltics, flora modifiers, intestinal adsorbents, intestinal anti-infectives, antianorexic, anticachexic, antibulimics, appetite suppressants, antiobesity agents, antacids, upper gastrointestinal tract agents, anticholinergic agents, aminosalicylic acid derivatives, biological response modifiers, corticosteroids, antispasmodics, 5-HT4 partial agonists, antihistamines, cannabinoids, dopamine antagonists, serotonin antagonists, cytoprotectives, histamine H2-receptor antagonists, mucosal protective agent, proton pump inhibitors, H. pylori eradication therapy, erythropoieses stimulants, hematopoietic agents, anemia agents, heparins, antifibrinolytics, hemostatics, blood coagulation factors, adenosine diphosphate inhibitors, glycoprotein receptor inhibitors, fibrinogen-platelet binding inhibitors, thromboxane-A₂ inhibitors, plasminogen activators, antithrombotic agents, glucocorticoids, mineralcorticoids, corticosteroids, selective immunosuppressive agents, antifungals, drugs involved in prophylactic therapy, AIDS-associated infections, cytomegalovirus, non-nucleoside reverse transcriptase inhibitors, nucleoside analog reverse transcriptse inhibitors, protease inhibitors, anemia, Kaposi's sarcoma, aminoglycosides, carbapenems, cephalosporins, glycopeptides, lincosamides, macrolies, oxazolidinones, penicillin, streptogramins, sulfonamides, trimethoprim and derivatives, tetracyclines, anthelmintics, amebicies, biguanides, cinchona alkaloids, folic acid antagonists, quinoline derivatives, Pneumocystis carinii therapy, hydrazides, imidazoles, triazoles, nitroimidzaoles, cyclic amines, neuraminidase inhibitors, nucleosides, phosphate binders, cholinesterase inhibitors, adjunctive therapy, barbiturates and derivatives, benzodiazepines, gamma aminobutyric acid derivatives, hydantoin derivatives, iminostilbene derivatives, succinimide derivatives, anticonvulsants, ergot alkaloids, antimigrane preparations, biological response modifiers, carbamic acid eaters, tricyclic derivatives, depolarizing agents, nondepolarizing agents, neuromuscular paralytic agents, CNS stimulants, dopaminergic reagents, monoamine oxidase inhibitors, COMT inhibitors, alkyl sulphonates, ethylenimines, imidazotetrazines, nitrogen mustard analogs, nitrosoureas, platinum-containing compounds, antimetabolites, purine analogs, pyrimidine analogs, urea derivatives, antracyclines, actinomycinds, camptothecin derivatives, epipodophyllotoxins, taxanes, vinca alkaloids and analogs, antiandrogens, antiestrogens, nonsteroidal aromatase inhibitors, protein kinase inhibitor antineoplastics, azaspirodecanedione derivatives, anxiolytics, stimulants, monoamind reuptake inhibitors, selective serotonin reuptake inhibitors, antidepressants, benzisooxazole derivatives, butyrophenone derivatives, dibenzodiazepine derivatives, dibenzothiazepine derivatives, diphenylbutylpiperidine derivatives, phenothiazines, thienobenzodiazepine derivatives, thioxanthene derivatives, allergenic extracts, nonsteroidal agents, leukotriene receptor antagonists, xanthines, endothelin receptor antagonist, prostaglandins, lung surfactants, mucolytics, antimitotics, uricosurics, xanthine oxidase inhibitors, phosphodiesterase inhibitors, methenamine salts, nitrofuran derivatives, quinolones, smooth muscle relaxants, parasympathomimetic agents, halogenated hydrocarbons, esters of amino benzoic acid, amides (e.g. lidocaine, articaine hydrochloride, bupivacaine hydrochloride), antipyretics, hypnotics and sedatives, cyclopyrrolones, pyrazolopyrimidines, nonsteroidal anti-inflammatory drugs, opioids, para-aminophenol derivatives, alcohol dehydrogenase inhibitor, heparin antagonists, adsorbents, emetics, opioid antagonists, cholinesterase reactivators, nicotine replacement therapy, vitamin A analogs and antagonists, vitamin B analogs and antagonists, vitamin C analogs and antagonists, vitamin D analogs and antagonists, vitamin E analogs and antagonists, vitamin K analogs and antagonists.

The antigen-binding proteins of the present disclosure can be conjugated to one or more cytokines and growth factors that are effective in inhibiting tumor metastasis, and wherein the cytokine or growth factor has been shown to have an antiproliferative effect on at least one cell population. Such cytokines, lymphokines, growth factors, or other hematopoietic factors include, but are not limited to: M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IFN, TNFα, TNF1, TNF2, G-CSF, Meg-CSF, GM-CSF, thrombopoietin, stem cell factor, and erythropoietin. Additional growth factors for use herein include angiogenin, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor α, cytokine-induced neutrophil chemotactic factor 1, cytokine-induced neutrophil, chemotactic factor 2 α, cytokine-induced neutrophil chemotactic factor 2 β, β endothelial cell growth factor, endothelin 1, epithelial-derived neutrophil attractant, glial cell line-derived neutrophic factor receptor α 1, glial cell line-derived neutrophic factor receptor α 2, growth related protein, growth related protein α, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neurotrophin-3, neurotrophin-4, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof.

The present disclosure also provides conjugates comprising an antigen-binding protein of the present disclosure linked to a polypeptide, such that the conjugate is a fusion protein. Therefore, the present disclosure provides fusion proteins comprising an antigen-binding protein of the present disclosure linked to a polypeptide. In various embodiments, the polypeptide is a diagnostic label, e.g., a fluorescent protein, such as green fluorescent protein, or other tag, e.g., Myc tag. In various aspects, the polypeptide is one of the cytokines, lymphokines, growth factors, or other hematopoietic factors listed above.

The present disclosure also provides conjugates comprising an antigen-binding protein of the present disclosure linked to a polypeptide, such that the conjugate is a fusion protein. Therefore, the present disclosure provides fusion proteins comprising an antigen-binding protein of the present disclosure linked to a polypeptide. In various embodiments, the polypeptide is a diagnostic label, e.g., a fluorescent protein, such as green fluorescent protein, or other tag, e.g., Myc tag. In various aspects, the polypeptide is one of the cytokines, lymphokines, growth factors, or other hematopoietic factors listed above.

The present disclosure also provides conjugates comprising an antigen-binding protein of the present disclosure linked to a polypeptide, such that the conjugate is a fusion protein. Therefore, the present disclosure provides fusion proteins comprising an antigen-binding protein of the present disclosure linked to a polypeptide. In various embodiments, the polypeptide is a diagnostic label, e.g., a fluorescent protein, such as green fluorescent protein, or other tag, e.g., Myc tag. In various aspects, the polypeptide is one of the cytokines, lymphokines, growth factors, or other hematopoietic factors listed above.

### P. Compositions and Formulations

Compositions comprising an FR2 region, an FW2/HV2 region, a VH domain, a VL domain, a VHH domain, a VNAR domain, an antigen-binding protein, a chimeric antigen receptor, a nucleic acid, a vector, a cell, or a conjugate as presently disclosed are provided herein.

The compositions in some aspects comprise the antigen-binding proteins in isolated and/or purified form. In some aspects, the composition comprises a single type (e.g., structure) of an antigen-binding protein of the present disclosure or comprises a combination of two or more antigen-binding proteins of the present disclosure, wherein the combination comprises two or more antigen-binding proteins of different types (e.g., structures).

In some aspects, the composition comprises agents which enhance the chemico-physico features of the antigen-binding protein, e.g., via stabilizing the antigen-binding protein at certain temperatures, e.g., room temperature, increasing shelf life, reducing degradation, e.g., oxidation protease mediated degradation, increasing half-life of the antigen-binding protein, etc. In some aspects, the composition comprises any of the agents disclosed herein as a heterologous moiety or conjugate moiety, optionally in admixture with the antigen-binding proteins of the present disclosure or conjugated to the antigen-binding proteins.

In various aspects of the present disclosure, the composition additionally comprises a pharmaceutically acceptable carrier, diluents, or excipient. In some embodiments, the antigen-binding protein, a nucleic acid, a vector, a cell, or a conjugate as presently disclosed (hereinafter referred to as “active agents”) is formulated into a pharmaceutical composition comprising the active agent, along with a pharmaceutically acceptable carrier, diluent, or excipient. In this regard, the present disclosure further provides pharmaceutical compositions comprising an active agent which is intended for administration to a subject, e.g., a mammal.

In some embodiments, the active agent is present in the pharmaceutical composition at a purity level suitable for administration to a patient. In some embodiments, the active agent has a purity level of at least about 90%, about 91%, about 92%, about 93%, about 94%, about 95%, about 96%, about 97%, about 98% or about 99%, and a pharmaceutically acceptable diluent, carrier or excipient. In some embodiments, the compositions contain an active agent at a concentration of about 0.001 to about 30.0 mg/ml.

In various aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The pharmaceutical composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

In various aspects, the pharmaceutical composition comprises formulation materials that are nontoxic to recipients at the dosages and concentrations employed. In specific embodiments, pharmaceutical compositions comprising an active agent and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In various aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants.

The pharmaceutical compositions can be formulated to achieve a physiologically compatible pH. In some embodiments, the pH of the pharmaceutical composition can be for example between about 4 or about 5 and about 8.0 or about 4.5 and about 7.5 or about 5.0 to about 7.5. In various embodiments, the pH of the pharmaceutical composition is between about 5.5 and about 7.5.

The present disclosure provides methods of producing a pharmaceutical composition. In various aspects, the method comprises combining the antigen-binding protein, conjugate, fusion protein, nucleic acid, vector, cell, or a combination thereof, with a pharmaceutically acceptable carrier, diluent, and/or excipient.

### Q. Composition/Formulation Administration

The active agent construct, or pharmaceutical composition comprising the same, can be administered to the subject via any suitable route of administration. For example, the active agent can be administered to a subject via parenteral, nasal, oral, pulmonary, topical, vaginal, or rectal administration. The following discussion on routes of administration is merely provided to illustrate various embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The active agent of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropyl methylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isosteric acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments contain from about 0.5% to about 25% by weight of the active agent of the present disclosure in solution. Preservatives and buffers can be used. In order to minimize or eliminate irritation at the site of injection, such compositions can contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are in accordance with the present disclosure. The requirements for effective pharmaceutical carriers for injectable compositions are well-known to those of ordinary skill in the art.

In some embodiments, the active agents or constructs are believed to be useful in methods of inhibiting tumor growth, as well as other methods, as further described herein, including methods of treating or preventing cancer.

The amount or dose of the active agent administered should be sufficient to effect, e.g., a therapeutic or prophylactic response, in the subject or animal over a reasonable time frame. For example, the dose of the active agent of the present disclosure should be sufficient to treat cancer as described herein in a period of from about 1 to 4 minutes, 1 to 4 hours or 1 to 4 weeks or longer, e.g., 5 to 20 or more weeks, from the time of administration. In certain embodiments, the time period could be even longer. The dose will be determined by the efficacy of the particular active agent and the condition of the animal (e.g., human), as well as the body weight of the animal (e.g., human) to be treated.

Many assays for determining an administered dose are known in the art. For purposes herein, an assay, which comprises comparing the extent to which cancer is treated upon administration of a given dose of the active agent of the present disclosure to a mammal among a set of mammals, each set of which is given a different dose of the active agent, could be used to determine a starting dose to be administered to a mammal. The extent to which cancer is treated upon administration of a certain dose can be represented by, for example, the extent of tumor regression achieved with the active agent in a mouse xenograft model. Methods of assaying tumor regression are known in the art and described herein in the Examples.

The dose of the active agent of the present disclosure also will be determined by the existence, nature and extent of any adverse side effects that might accompany the administration of a particular active agent of the present disclosure. Typically, the attending physician will decide the dosage of the active agent of the present disclosure with which to treat each individual patient, taking into consideration a variety of factors, such as age, body weight, general health, diet, sex, active agent of the present disclosure to be administered, route of administration, and the severity of the condition being treated. By way of example and not intending to limit the present disclosure, the dose of the active agent of the present disclosure can be about 0.0001 to about 1 g/kg body weight of the subject being treated/day, from about 0.0001 to about 0.001 g/kg body weight/day, or about 0.01 mg to about 1 g/kg body weight/day.

In other embodiments, controlled release formulations may be provided. For example, the active agents described herein can be modified into a depot form, such that the manner in which the active agent of the present disclosure is released into the body to which it is administered is controlled with respect to time and location within the. Depot forms of active agents of the present disclosure can be, for example, an implantable composition comprising the active agents and a porous or non-porous material, such as a polymer, wherein the active agent is encapsulated by or diffused throughout the material and/or degradation of the non-porous material. The depot is then implanted into the desired location within the body of the subject and the active agent is released from the implant at a predetermined rate.

The pharmaceutical composition comprising the active agent in certain aspects is modified to have any type of in vivo release profile. In some aspects, the pharmaceutical composition is an immediate release, controlled release, sustained release, extended release, delayed release, or bi-phasic release formulation. Methods of formulating peptides for controlled release are known in the art.

The instant compositions can further comprise, for example, micelles or liposomes, or some other encapsulated form, or can be administered in an extended-release form to provide a prolonged storage and/or delivery effect.

### R. Compositions Preventing or Treating Diseases

The pharmacological compositions of the present disclosure are useful in treating various diseases including but not limited to a cancer, an inflammatory disease, an infection (e.g., a viral infection (e.g., SARS virus, HIV virus, influenza virus), a bacterial infection (e.g., targeting bacteria or their toxins), or a fungal infection), neurological disorders, musculoskeletal disorders (e.g., osteoarthritis, rheumatoid arthritis, psoriatic arthritis, gout, ankylosing spondylitis, osteoporosis, osteopenia, sarcopenia, systemic lupus erythematosus, carpal tunnel syndrome, fibromyalgia), ophthalmology diseases (e.g., Retinitis Pigmentosa, Age-Related Macular Degeneration, Glaucoma, Diabetic Retinopathy, Strabismus, Uveitis, Thyroid Eye Disease-Graves' Disease, Optic Neuritis, Retinopathy of Prematurity (ROP), Cataracts, Retinoblastoma), genetic diseases, hematological disorders (e.g., anemia, conditions related to HIV, sickle cell disease, and complications from chemotherapy or transfusions), or high cholesterol (e.g., use of Evinacumab or a similar antigen-binding protein). The inherent modularity and flexibility of the ODIN platform technology make it suitable across multiple diverse indications. Oncology currently represents 40% of the immunotherapeutic market. In this space, the ODIN molecules with a small size (e.g., sdAb) have the advantage, because they: (i) enhance penetration of solid tumors and (ii) afford tighter cell-cell synapses bridging effector and target cells (e.g., cytotoxic T cells and tumor cells). For infectious disease, the size advantage of small ODINs should also engender its enhanced biodistribution into reservoirs of viral replication, allowing more rapid shutoff of viral reservoirs in tissues and organs and extending the window of treatment beyond that of classic IgG-based immunotherapeutics. For neurological disorders, the small size of the ODIN molecules (the antigen-binding protein of the present disclosure) enables them to cross the blood-brain barrier and facilitates targeting of the brain antigens.

In certain aspects, provided herein is a method of preventing or treating an inflammatory disease in a subject, the method comprising administering to the subject at least one engineered antigen-binding protein or a pharmaceutical composition of the present disclosure.

In certain aspects, provided herein is a method of preventing or treating an infection in a subject, e.g., a viral infection (e.g., SARS-CoV-2 and/or an HIV infection, e.g., HIV-1), a bacterial infection, or a fungal infection, the method comprising administering to the subject at least one engineered antigen-binding protein or a pharmaceutical composition of the present disclosure.

In certain aspects, provided herein is a method of preventing or treating a cancer in a subject, the method comprising administering to the subject at least one engineered antigen-binding protein or a pharmaceutical composition of the present disclosure.

In certain aspects, also provided herein is a method of reducing proliferation of a cancer cell in a subject, the method comprising administering to the subject at least one engineered antigen-binding protein or a pharmaceutical composition of the present disclosure.

Further provided herein are methods of inhibiting tumor growth in a subject and methods of reducing tumor size in a subject. In various embodiments, the method comprising administering to the subject at least one engineered antigen-binding protein or a pharmaceutical composition of the present disclosure in an amount effective for inhibiting tumor growth or reducing tumor size in the subject.

In certain embodiments, the therapeutically effective amount of an engineered antigen-binding protein or pharmaceutical composition is administered to a subject in need thereof. In other embodiments, the cells that are autologous or allogeneic to the subject are obtained and transduced (e.g., via a viral vector, such as AAV) or otherwise transformed with a nucleic acid (or a vector comprising same) that encodes any one of the engineered antigen-binding protein of the present disclosure. In preferred embodiments, such nucleic acid is stably integrated into the cell genome. Upon confirming transformation of the nucleic acid, the cells are introduced to the subject (e.g., grafted or implanted) to supply a continued source of the antigen-binding proteins (i.e., expressed by the grafted cells and secreted into blood).

As used herein, the term “inhibit” or “reduce” and words stemming therefrom may not be a 100% or complete inhibition or reduction. Rather, there are varying degrees of inhibition or reduction of which one of ordinary skill in the art recognizes as having a potential benefit or therapeutic effect. In this respect, the antigen-binding proteins of the present disclosure may inhibit tumor growth or reduce tumor size to any amount or level. In various embodiments, the inhibition provided by the methods of the present disclosure is at least or about a 10% inhibition (e.g., at least or about a 20% inhibition, at least or about a 30% inhibition, at least or about a 40% inhibition, at least or about a 50% inhibition, at least or about a 60% inhibition, at least or about a 70% inhibition, at least or about a 80% inhibition, at least or about a 90% inhibition, at least or about a 95% inhibition, at least or about a 98% inhibition). In various embodiments, the reduction provided by the methods of the present disclosure is at least or about a 10% reduction (e.g., at least or about a 20% reduction, at least or about a 30% reduction, at least or about a 40% reduction, at least or about a 50% reduction, at least or about a 60% reduction, at least or about a 70% reduction, at least or about a 80% reduction, at least or about a 90% reduction, at least or about a 95% reduction, at least or about a 98% reduction).

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the method of the present disclosure can include treatment of one or more conditions or symptoms or signs of the cancer being treated. Also, the treatment provided by the methods of the present disclosure can encompass slowing the progression of the cancer. In various aspects, the methods treat by way of delaying the onset or recurrence of the cancer by at least 1 day, 2 days, 4 days, 6 days, 8 days, 10 days, 15 days, 30 days, two months, 3 months, 4 months, 6 months, 1 year, 2 years, 3 years, 4 years, or more. In various aspects, the methods treat by way increasing the survival of the subject.

In some embodiments, the method further comprises conjointly administering to the subject an additional cancer therapy. In some embodiments, the additional cancer therapy is selected from the group consisting of immunotherapy, checkpoint blockade, cancer vaccines, chimeric antigen receptors, chemotherapy, radiation, target therapy, and surgery.

### 1. Cancer

Cancer, tumor, or hyperproliferative disorder refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like.

Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma (SCLC), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

In some embodiments, the cancer is selected from pancreatic cancer, lung cancer, non-small cell lung cancer (NSCLC), malignant pleural mesothelioma, small cell lung cancer (SCLC), renal cell carcinoma (RCC), breast cancer, liver cancer, hepatocellular carcinoma, kidney cancer, skin cancer, melanoma, thyroid cancer, gall bladder cancer, head-and-neck (squamous) cancer, stomach (gastric) cancer, head and neck cancer, bladder cancer, urothelial carcinoma, Merkel cell cancer, colon cancer, colorectal cancer, intestinal cancer, ovarian cancer, cervical cancer, testicular cancer, esophageal cancer, buccal cancer, brain cancer, blood cancers, lymphomas (B and T cell lymphomas), mesothelioma, cutaneous squamous cell cancer, Hodgkin's lymphoma, B-cell lymphoma, and a malignant or metastatic form thereof.

The therapeutic agents of the present disclosure can be used alone or can be administered in combination/conjoint therapy with, e.g., chemotherapeutic agents, hormones, antiangiogens, radiolabeled, compounds, or with surgery, cryotherapy, immunotherapy, cancer vaccine, immune cell engineering (e.g., CAR-T), and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, agents of the present disclosure can be administered with a therapeutically effective dose of chemotherapeutic agent. In other embodiments, agents of the present disclosure are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician.

Immunotherapy is a targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF is known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy also encompasses immune checkpoint modulators. Immune checkpoints are a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, TMIDG2, KIR3DL3, and A2aR. Inhibition of one or more immune checkpoint inhibitors can block or otherwise neutralize inhibitory signaling to thereby upregulate an immune response in order to more efficaciously treat cancer. In some embodiments, the cancer therapy one or more inhibitors of immune checkpoints (immune checkpoint inhibition therapy), such as PD1, PD-1, and/or CD47 inhibitors. In some embodiments, the cancer therapy is nivolumab.

Adoptive cell-based immunotherapies can be combined with the therapies of the present disclosure. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like.

The term “chimeric antigen receptor” or “CAR” refers to engineered T cell receptors (TCR) having a desired antigen specificity. T lymphocytes recognize specific antigens through interaction of the T cell receptor (TC R) with short peptides presented by major histocompatibility complex (MHC) class I or II molecules. For initial activation and clonal expansion, naive T cells are dependent on professional antigen-presenting cells (APCs) that provide additional co-stimulatory signals. TCR activation in the absence of co-stimulation can result in unresponsiveness and clonal anergy. To bypass immunization, different approaches for the derivation of cytotoxic effector cells with grafted recognition specificity have been developed. CARs have been constructed that consist of binding domains derived from natural ligands or antibodies specific for cell-surface components of the TCR-associated CD3 complex. Upon antigen binding, such chimeric antigen receptors link to endogenous signaling pathways in the effector cell and generate activating signals similar to those initiated by the TCR complex. Since the first reports on chimeric antigen receptors, this concept has steadily been refined and the molecular design of chimeric receptors has been optimized and routinely use any number of well-known binding domains, such as scFV and another protein binding fragments described herein.

In other embodiments, immunotherapy comprises non-cell-based immunotherapies. In some embodiments, compositions comprising antigens with or without vaccine-enhancing adjuvants are used. Such compositions exist in many well-known forms, such as peptide compositions, oncolytic viruses, recombinant antigen comprising fusion proteins, and the like. In some embodiments, immunomodulatory cytokines, such as interferons, G-CSF, imiquimod, TNFalpha, and the like, as well as modulators thereof (e.g., blocking antibodies or more potent or longer lasting forms) are used. In some embodiments, immunomodulatory interleukins, such as IL-2, IL-6, IL-7, IL-12, IL-17, IL-23, and the like, as well as modulators thereof (e.g., blocking antibodies or more potent or longer lasting forms) are used. In some embodiments, immunomodulatory chemokines, such as CCL3, CCL26, and CXCL7, and the like, as well as modulators thereof (e.g., blocking antibodies or more potent or longer lasting forms) are used. In some embodiments, immunomodulatory molecules targeting immunosuppression, such as STAT3 signaling modulators, NFkappaB signaling modulators, and immune checkpoint modulators, are used.

In still other embodiments, immunomodulatory drugs, such as immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, zotarolimus, etc.), hydrocortisone (cortisol), cortisone acetate, prednisone, prednisolone, methylprednisolone, dexamethasone, betamethasone, triamcinolone, beclometasone, fludrocortisone acetate, deoxycorticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-xB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), I3C(indole-3-carbinol)/DIM(di-indolmethane) (13C/DIM), Bay 11-7082, luteolin, cell permeable peptide SN-50, IKBa.-super repressor overexpression, NFKB decoy oligodeoxynucleotide (0 DN), or a derivative or analog of any thereof, are used. In yet other embodiments, immunomodulatory antibodies or protein are used. For example, antibodies that bind to CD40, Toll-like receptor (TLR), OX40, GITR, CD27, or to 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 an antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40 antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-eotaxin 1 antibody, bertilimumab, an anti-a4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, talizumab, an IL12 inhibitor, an IL23 inhibitor, ustekinumab, and the like.

Nutritional supplements that enhance immune responses, such as vitamin A, vitamin E, vitamin C, and the like, are well-known in the art and can be used in the methods described herein. Similarly, various agents or a combination thereof can be used to treat a cancer. For example, chemotherapy, radiation, epigenetic modifiers (e.g., histone deacetylase (HDAC) modifiers, methylation modifiers, phosphorylation modifiers, and the like), targeted therapy, and the like are well-known in the art.

In some embodiments, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof.

Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiments, PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ 34; 3-aminobenzamide (Trevigen); 4-amino-1,8-naphthalimide; (Trevigen); 6(5H)-phenanthridinone (Trevigen); benzamide; and NU1025. The mechanism of action is generally related to the ability of PARP inhibitors to bind PARP and decrease its activity. PARP catalyzes the conversion of .beta.-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis. The foregoing examples of chemotherapeutic agents are illustrative and are not intended to be limiting.

In other embodiments, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In other embodiments, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In other embodiments, photodynamic therapy (also called PDT, photo radiation therapy, phototherapy, or photochemotherapy) is used for the treatment of some types of cancer. It is based on the discovery that certain chemicals known as photosensitizing agents can kill one-celled organisms when the organisms are exposed to a particular type of light.

In yet other embodiments, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors.

Clinical efficacy can be measured by any method known in the art. For example, the response to a therapy relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (P R) and the number of patients having stable disease (SD) at a time point at least 6 months out from the end of therapy. The shorthand for this formula is CBR=CR+PR+SD over 6 months. In some embodiments, the CBR fora particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to a cancer therapy are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following the cancer therapy for whom biomarker measurement values are known. In certain embodiments, the same doses of anti-cancer agents are administered to each subject. In related embodiments, the doses administered are standard doses known in the art for anti-cancer agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate with the outcome of a cancer therapy can be determined using methods such as those described in the Examples section.

### 2. Inflammatory Disorder Therapies

The engineered antigen-binding proteins and/or pharmaceutical compositions described herein can be used, for example, for preventing or treating (reducing, partially or completely, the adverse effects of) an inflammatory disease, such as chronic inflammatory bowel disease, systemic lupus erythematosus, psoriasis, muckle-wells syndrome, rheumatoid arthritis, multiple sclerosis, or Hashimoto's disease, an allergic disease, asthma; an infectious disease; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease. The engineered antigen-binding proteins and/or pharmaceutical compositions can be used for suppressing rejection in organ transplantation or other situations in which tissue rejection might occur; for improving immune functions; or for suppressing the proliferation or function of immune cells.

In certain embodiments, the inflammatory disorders include inflammation of any tissue and organs of the body, including musculoskeletal inflammation, vascular inflammation, neural inflammation, digestive system inflammation, ocular inflammation, inflammation of the reproductive system, and other inflammation.

In some embodiments, the musculoskeletal inflammation includes conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

In some embodiments, the ocular immune disorder refers to an immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis.

In some embodiments, the nervous system immune disorders which may be treated with the methods and compositions described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

In some embodiments, the digestive system immune disorders which may be treated with the methods and pharmaceutical compositions described herein include cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

In some embodiments, the reproductive system immune disorders which may be treated with the methods and pharmaceutical compositions described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

In some embodiments, the inflammatory disorders include acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, type 2 diabetes, giant cell arteritis, Goodpasture's syndrome, Graves' disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and compositions described herein may be used to treat T-cell mediated hypersensitivity diseases having an inflammatory component. Such conditions include contact hypersensitivity, contact dermatitis (including that due to poison ivy), urticaria, skin allergies, respiratory allergies (hay fever, allergic rhinitis, house dust mite allergy) and gluten-sensitive enteropathy (Celiac disease).

Other immune disorders which may be treated with the methods and pharmaceutical compositions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, and stomatitis, transplant rejection (involving organs such as kidney, liver, heart, lung, pancreas (e.g., islet cells), bone marrow, cornea, small bowel, skin allografts, skin homografts, and heart valve xenografts, serum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autoimmune) haemolytic anemia, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (e.g., sepsis).

### 3. Neurological Disease Therapy

In some embodiments, the neurological diseases include but not limited to Demyelinating Diseases (e.g., Multiple Sclerosis); delirium and dementia (e.g., vascular dementia, dementia due to Parkinson's disease, dementia due to HIV disease, dementia due to Huntington's disease, and dementia due to Creutzfeldt-J akob disease; Alzheimer's dementia, multi-infarct dementia, stroke); affective disorder (e.g., depression, mania, mood disorder, major depressive disorder, bipolar); movement disorders (e.g., restless leg syndrome, Dyskinesia (e.g., tremor, dystonia, chorea and ballism, tic syndromes (e.g., Tourette's Syndrome), myoclonus, drug-induced movement disorders, Wilson's Disease, Paroxysmal Dyskinesias, Stiff Man Syndrome) and Akinetic-Rigid Syndromes and Parkinsonism); ataxic disorders (e.g., disturbances of gait); personality disorders (e.g., schizoid personality disorder, paranoid personality disorder, schizotypal personality disorder, borderline personality disorder, narcissistic personality disorder, histrionic personality disorder, obsessive compulsive personality disorder, avoidant personality disorder, dependent personality disorder, and anti-social personality disorder); and other psychiatric disorders (e.g., schizophrenia subtypes, schizoaffective disorder, schizophrenia undifferentiated, delusional disorder, cyclothymic disorder, somatoform disorder, hypochondriasis, dissociative disorder, and depersonalization disorder).

Neurodegeneration is the progressive loss of structure or function of neurons, including death of neurons. Many neurodegenerative diseases—including amyotrophic lateral sclerosis, Parkinson's disease, Alzheimer's disease, and Huntington's disease—occur as a result of neurodegenerative processes. Such diseases are incurable, resulting in progressive degeneration and/or death of neuron cells. As research progresses, many similarities appear that relate these diseases to one another on a sub-cellular level. Discovering these similarities offers hope for therapeutic advances that could ameliorate many diseases simultaneously.

### S. Kits

In some embodiments, an FR2 region, an FW2/HV2 region, a VH domain, a VL domain, a VHH domain, a VNAR domain, an antigen-binding protein, a chimeric antigen receptor, a nucleic acid, a vector, a cell, a conjugate, or pharmaceutical compositions of the present disclosure are provided in a kit. In various aspects, the kit comprises the antigen-binding protein(s) as a unit dose. For purposes herein “unit dose” refers to a discrete amount dispersed in a suitable carrier. In various aspects, the unit dose is the amount sufficient to provide a subject with a desired effect, e.g., inhibition of tumor growth, reduction of tumor size, treatment of cancer, treatment of infection, treatment of an inflammatory disease. Accordingly, provided herein are kits comprising an antigen-binding protein of the present disclosure optionally provided in unit doses. In various aspects, the kit comprises several unit doses, e.g., a week or month supply of unit doses, optionally, each of which is individually packaged or otherwise separated from other unit doses. In some embodiments, the components of the kit/unit dose are packaged with instructions for administration to a patient. In some embodiments, the kit comprises one or more devices for administration to a patient, e.g., a needle and syringe, and the like. In some aspects, the antigen-binding protein of the present disclosure, a pharmaceutically acceptable salt thereof, a conjugate comprising the antigen-binding protein, or a multimer or dimer comprising the antigen-binding protein, is pre-packaged in a ready to use form, e.g., a syringe, an intravenous bag, etc. In some aspects, the kit further comprises other therapeutic or diagnostic agents or pharmaceutically acceptable carriers (e.g., solvents, buffers, diluents, etc.), including any of those described herein. In particular aspects, the kit comprises an antigen-binding protein of the present disclosure, along with an agent, e.g., another therapeutic agent that treats the disease.

The technology described herein may be better understood with reference to the accompanying examples, which are intended for purposes of illustration only and should not be construed as in any sense limiting the scope of the technology described herein as defined in the claims appended hereto.

### Example 1

In order to demonstrate the operational principles of the technology, a system using rVACV display vectors encoding VHHs and ODINs was designed and validated. VHH and ODIN protein-coding sequences fused to A56R (SEQ005-008 above) were cloned into the NotI and BamHI sites of a transfer vector to position them downstream of the VACV synthetic E L promoter (PMID: 9400616) and between flanking sequences (˜300 bp on each side) homologous for the thymidine kinase (tk) locus. Transfer amplicons were generated from these plasmids by PCR with terminal primers and rVACVs bearing each VHH expression cassette in the tk locus were rescued by MAVERICC, as described previously (P MID: 33639215).

Large-scale isolation of VACV extracellular enveloped virions (EEVs) displaying VHHs and ODINs was also conducted. EEVs were generated by infection of BSC40 cells with rVACVs (MOI=0.1 PFU/cell). Cell supernatants were harvested at 72 h post-infection and ultracentrifuged through 36% sucrose cushions. EEV pellets were resuspended in NT buffer (140 mM NaCl, 10 mM Tris[pH 7.5]), normalized for protein content, and visualized by western blot with anti-EEV B5R and anti-HA tag antibodies.

EEV ELISAs for antigen binding were also prepared. EEVs (5 μL/well) were coated into high protein-binding ELISA plates. Plates were then blocked and incubated with serial dilutions of recombinant Strep-tagged SARS-CoV-2 trimeric spike (prepared as in PMID: 33458462) or biotinylated recombinant HIV-1 trimeric spike (BG505 SOSIP.664; gift of Dr. Andrew B. Ward, PMID: 29150937). Plates were washed, and SARS-CoV-2 and HIV-1 spike proteins were detected with streptactin-horseradish peroxidase (HRP) orstreptavidin-HRP conjugates, respectively.

For sandwich antigen-binding ELISAs, plates were coated with 50 ng non-biotinylated HIV-1 spike per well, followed by EEVs (5 μL), and serial dilutions of Strep-tagged SARS-CoV-2 spike. Binding by the latter was detected with streptactin-HRP.

The expression and purification of soluble recombinant VHHs and ODINs were evaluated. Plasmids for CMV promoter-driven expression of VHHs and ODINs (e.g., those comprising the amino acid sequences set forth in SEQ ID NOs: 13-16) were transfected into ExpiCHO cells in suspension culture and purified from cell supernatants by nickel-chelation chromatography, as described previously (P MID: 34721393). For biolayer interferometry (BLI) assays for antigen binding by recombinant VHHs and ODINs, biotinylated HIV-1 spike protein was loaded onto SAX streptavidin sensors (Octet, Sartorius). Sensors were then dipped in solutions containing different [VHH] and protein association overtime was monitored with the OctetRed system. Global fitting to a 1:1 binding model was used to estimate kon (association rate constant), koff (dissociation rate constant). KD (equilibrium dissociation constant) was calculated as koff/kon.

HIV-1 pseudotype neutralization assays were also conducted. Serially diluted ODIN proteins were incubated with a pre-titrated amount of HIV-1 BG505 Env-pseudotyped virus in growth media. Freshly trypsinized TZM-bl cells, which contain integrated firefly luciferase and E. coli B-galactosidase reporter genes under control of an HIV-1 long terminal repeat, were diluted in growth media with DEAE-Dextran before being added to protein: virus mixture and incubated for 48 hours. The cells were treated with Promega Bright-Glo luciferase reagent and luminescence was read using Cytation 5.

Recombinant vesicular stomatitis virus pseudotype neutralization assays were conducted. A dilution series of ODIN proteins were incubated with a pre-titrated amount of recombinant vesicular stomatitis viruses (rVSVs) encoding enhanced green fluorescent protein (eGFP) in the first position and replacing VSV G with glycoproteins from EBOV, SARS-CoV-2, or MERS-CoV for 1 hour at room temperature prior to addition onto Vero cell monolayers. Viral infectivities were measured by automated enumeration of eGFP+cells using a Cytation 5 reader at 12-14 h post-infection hours for rVSV-EBOV or 8 hours for rVSV-SAR S-CoV-2 and rVSV-MERS-CoV.

### Example 2

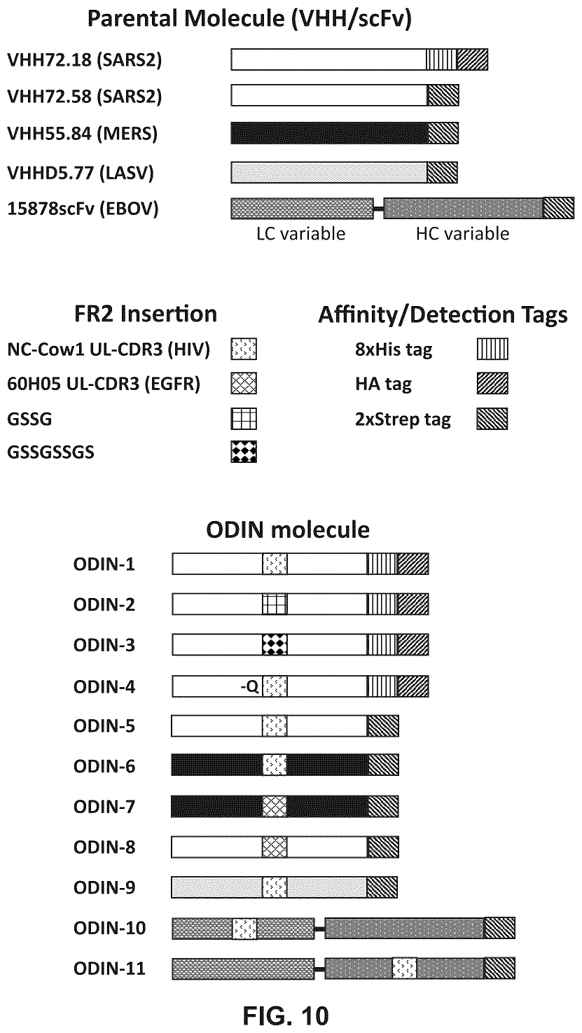

To further evaluate the functionality of the constructs, the, ODIN (Orthogonal Dual-Interacting Nanotherapeutic) bispecific scaffold was engineered. The ODIN construct, which combined the unique functional properties of conventional IgGs, VHHs, and bovine picobodies into a single well-folded domain using structure-based engineering, mutagenesis and molecular resurfacing (See FIG. 3A-FIG. 3C) was evaluated. The illustrated ODIN was the smallest sdAb-based bispecific molecule developed to date that does not require IgG-based glycosylated fragments produced in mammalian cells (e.g., BITEs, DARTs) that does not resort to a ‘beads-on-a-string approach’ to achieve bispecificity (e.g., BITEs, bispecific VHHs) (FIG. 2). The ODIN leveraged a previously unexplored site in the highly conserved VHH framework, the beta-hairpin formed by the C and C′ strands and the intervening loop (FIG. 3A), to fuse an engineered bovine picobody stalk topped by an antigen-specific picobody knob (FIG. 3B and FIG. 3C). Crucially, the beta strands comprising the stalk were fused in register with the C-C′ beta hairpin so that the picobody was predicted to extend seamlessly from the body of the VHH without any flexible loop regions. Multiple ODIN molecules were constructed as shown in FIG. 10 and evaluated to establish the feasibility of the ODIN concept.

### Example 3

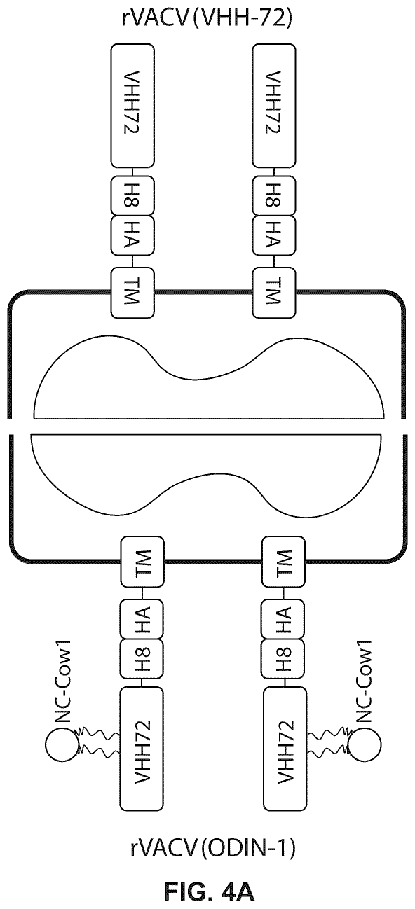

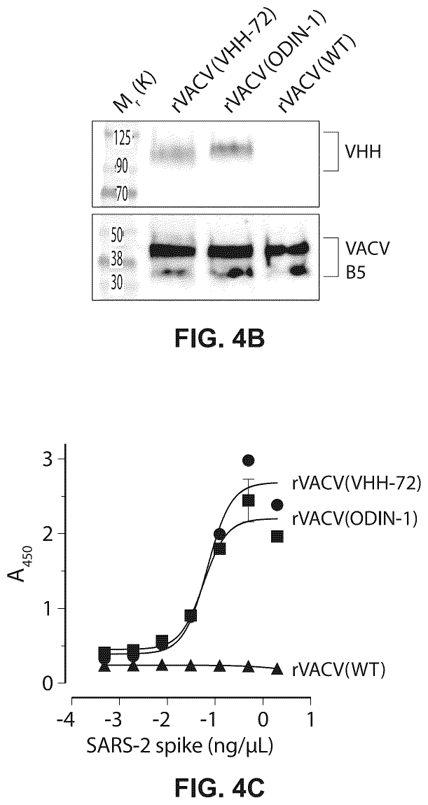

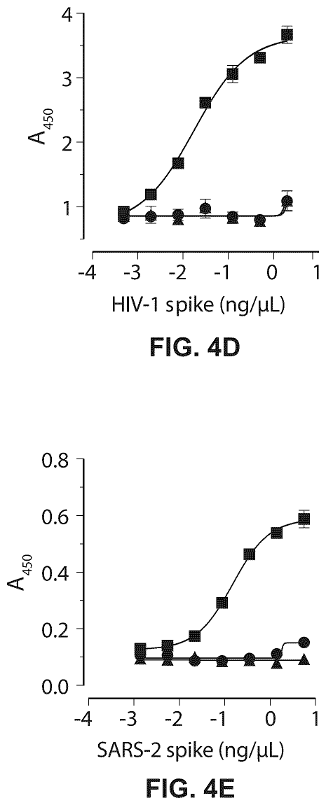

To further evaluate the fabrication processes and functionality of the compositions, bispecific VHH that binds SARS-CoV-2 and HIV-1 was produced and evaluated. To establish the feasibility of the ODIN concept, a VHH specific for the SARS-CoV-2 (SARS-2) spike protein (VHH-72) was engineered bearing a picobody specific for the HIV-1 spike trimer with a HIS tag (ODIN-1) and with a Strep tag (ODIN-5) (NC-Cowl). This construct, ODIN-1, could be displayed on the surface of recombinant vaccinia (rVACV) poxvirus particles (See FIG. 4A and FIG. 4B). Both rVACVs decorated with WT VHH-72 and ODIN-1 could capture a recombinant SARS-2 spike protein as shown in FIG. 4C, but only ODIN-1 could capture recombinant HIV-1 spike as seen in FIG. 4D.

Further, it was observed that ODIN-1, but not WT VHH-72, could bridge SARS-2 and HIV-1 spike proteins in a sandwich ELISA as demonstrated in FIG. 4E. This demonstrates that both antigen-binding sites in ODIN-1 are functional.

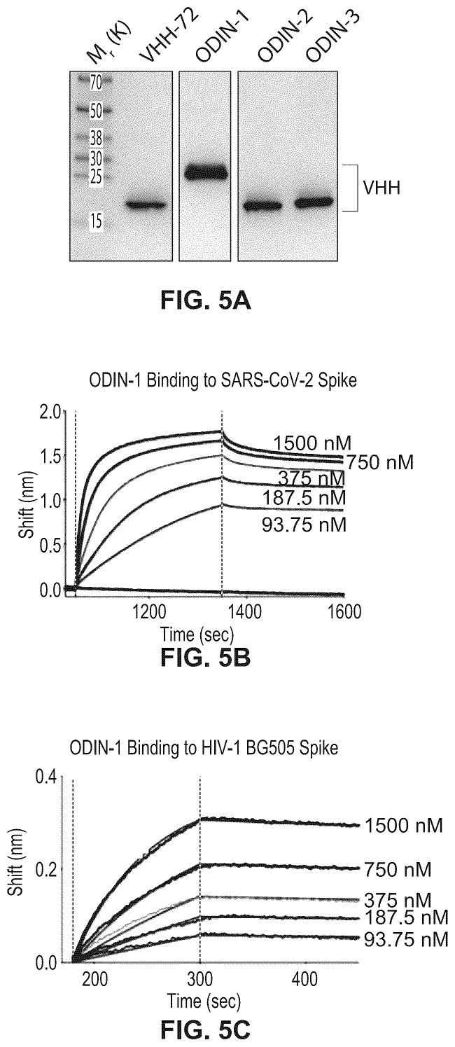

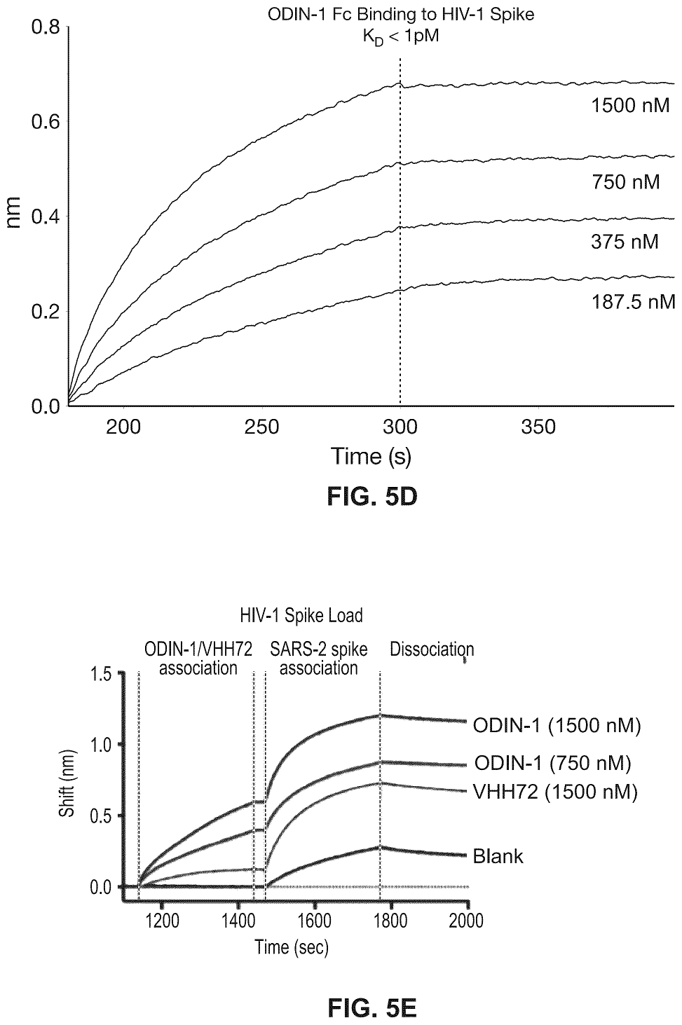

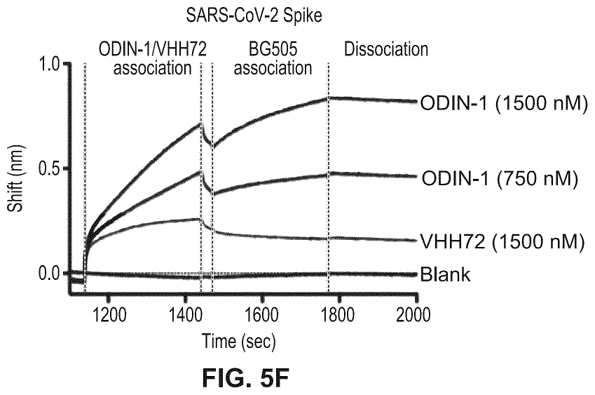

VHH-72 and ODIN-1 were next expressed as secreted proteins in ExpiCHO cell suspension cultures and purified them from cell supernatants by nickel-chelation chromatography (FIG. 5A). ODIN-1, bound to SARS-CoV-2 spike is shown in FIG. 5B and HIV-1 BG505 spike with high affinity (KDapp ˜30 nM) in a biolayer interferometry assay is shown in FIG. 5C, further validating the conclusions from the rVACV display experiments above.

Additionally, ODIN-1 was fused to an Fc region and shown to retain high affinity to the HIV-1 spike protein via BLI, demonstrating the presence of an Fc region does not disrupt the binding affinity of the inserted picobody as seen in FIG. 5D. While both paratopes retained high affinity to the individual ligands it remained to be determined if ODIN-1 could bind the SARS-CoV-2 spike and HIV-1 BG505 spike proteins simultaneously. Therefore, we also tested ODIN-1 in a sandwich BLI experiment (FIG. 5E and FIG. 5F), which demonstrated that small ODIN bispecifics can readily bind and bridge two large, heavily glycosylated proteins simultaneously.

Moreover, VHH-72 also tolerated the insertion of 6- and 10-amino acid glycine-serine linkers into the C-C′ loop of the VHH framework (ODIN-2 and ODIN-3; See FIG. 5A), indicating that ODIN variants bearing a broad range of variable peptide sequences (and not just bovine picobodies) can be generated.

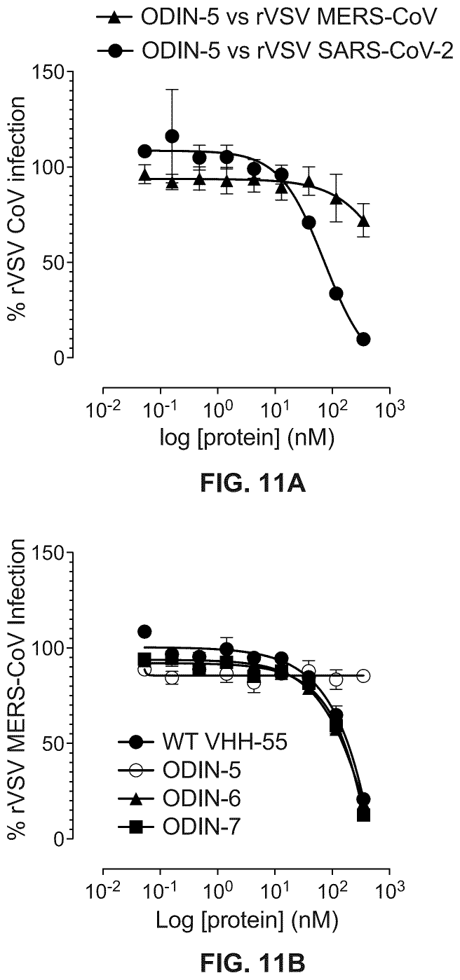

Further expanding on the ODIN-1 data, two additional VHHs were modified, VHH-55 which targets the Middle East respiratory syndrome coronavirus (MERS-CoV) spike protein and VHH.D5 which targets the Lassa virus (LASV) spike protein. VHH-55 and VHH.D5 were both engineered in the same way as ODIN-1 to insert a picobody specific for the HIV-1 spike trimer (NC-Cowl), to create ODIN-6 and ODIN-9. ODIN-5 (VHH72 with NC-Cowl pico) demonstrate the ability to neutralize SARS-CoV2 (See FIG. 11A) and MERS-CoV (See FIG. 11B), respectively, showing both ODIN molecules retain the functionality of the VHH component.

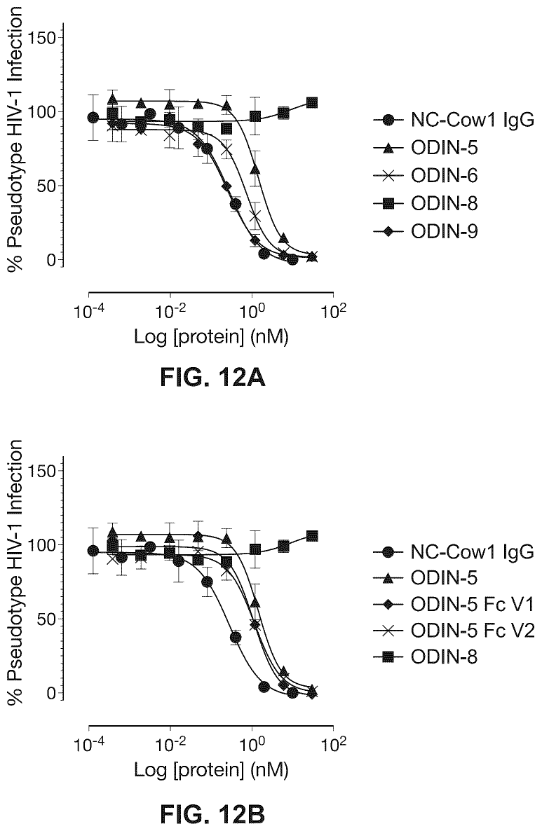

Additionally, ODIN-5 (a strep tagged version of ODIN-1), ODIN-6 and ODIN-9 all retained the ability to neutralize HIV-1 via the NC-Cowl picobody insertion (FIG. 12A), demonstrating the successful modification of three unrelated VHHs with a bovine picobody sequence within the C-C′ loop insertion site. The ODIN-5 was also engineered with an Fc region with two different hinge region linkers creating ODIN-5 Fc V1 and ODIN-5 Fc V2. Both ODIN-5 Fc V1 and V2 continued to neutralize HIV-1 (FIG. 12B), demonstrating that the presence of an Fc region on the c-terminus does not negatively impact the functionality of the ODIN NC-Cowl picobody insertion.

### Example 4

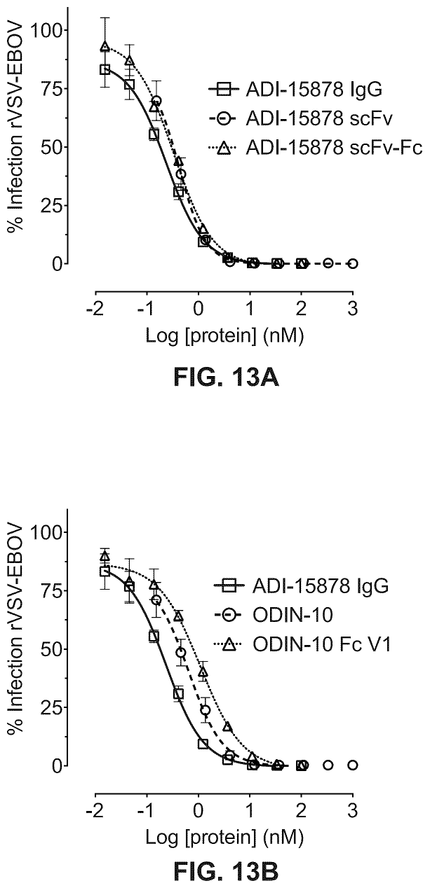

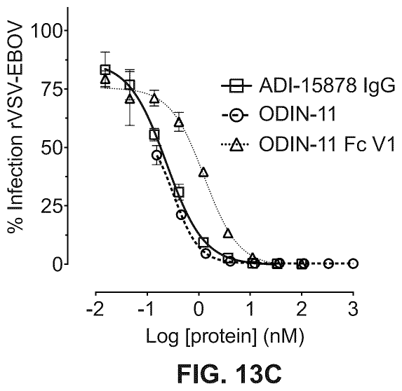

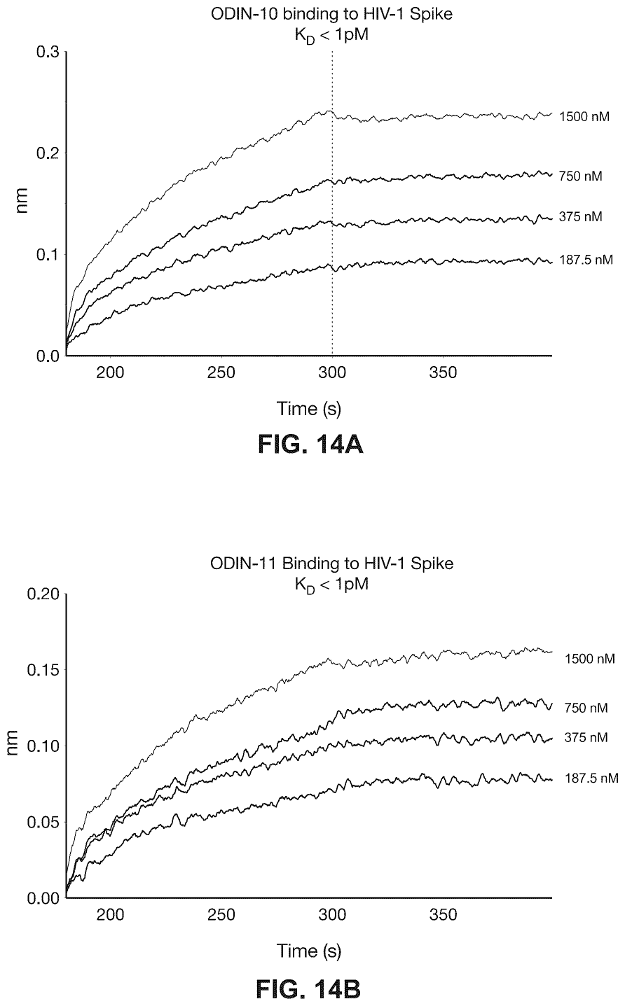

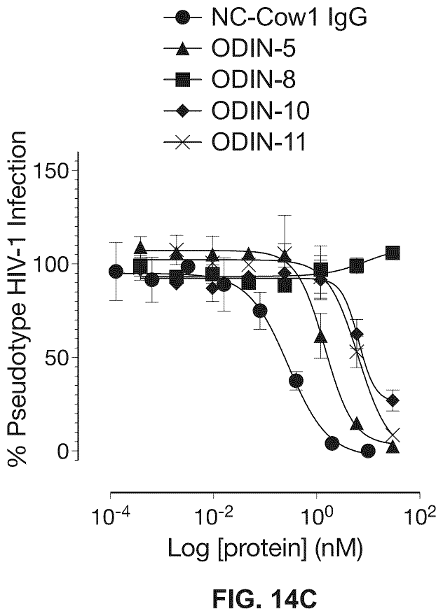

To further demonstrate the functionality of the constructs, a bispecific scFv that binds Ebolavirus and HIV-1 was fabricated and tested. In this example, the ODIN concept was applied to the human V_{H} and V_{L} domain from the ebolavirus (EBOV) antibody ADI-15878. A single-chain variable fragment (scFv) of ADI-15878 was created with and without and Fc region as seen in FIG. 10. The scFv and scFv-Fc were then shown to retain the ability to neutralize a rVSV pseudovirus displaying the EBOV spike protein (rVSV-EBOV) prior to modification as seen in FIG. 13A. ODIN molecules were then created by inserting the NC-Cowl picobody into the ADI-15878 VH, ODIN-10, and a version with an Fc region, ODIN-10 Fc V1. Another ODIN molecule, ODIN-11, was created by inserting the NC-Cowl picobody into the ADI-15878 VL, which was also fused to an Fc region making ODIN-11 Fc V1. All of these ODIN variants continued to effectively neutralize rVSV-EBOV (FIG. 13B and FIG. 13C). The functionality of the NC-Cowl picobody insertion confirmed by BLI affinity experiments showing both ODIN-10 and ODIN-11 maintain a high affinity for the HIV-1 spike protein (See FIG. 14A and FIG. 14B) and both neutralize HIV-1 pseudoviruses as shown in FIG. 14C. This demonstrated the fact that the ODIN concept can be readily applied to the equivalent insertion site in human VH and VL regions to create bispecific antibodies with and without a Fc region.

### Example 5

The functionality of the constructs was further demonstrated with the production of a bispecific VHH that binds SARS-CoV-2 or MERS-CoV and human epidermal growth factor receptor (EGFR). To further establish the feasibility of the ODIN concept, a different picobody specific to EGFR (60H05; Pekar et al. 2021) was inserted into VHH-55 or VHH-72, creating ODIN-7 and ODIN-8, respectively.

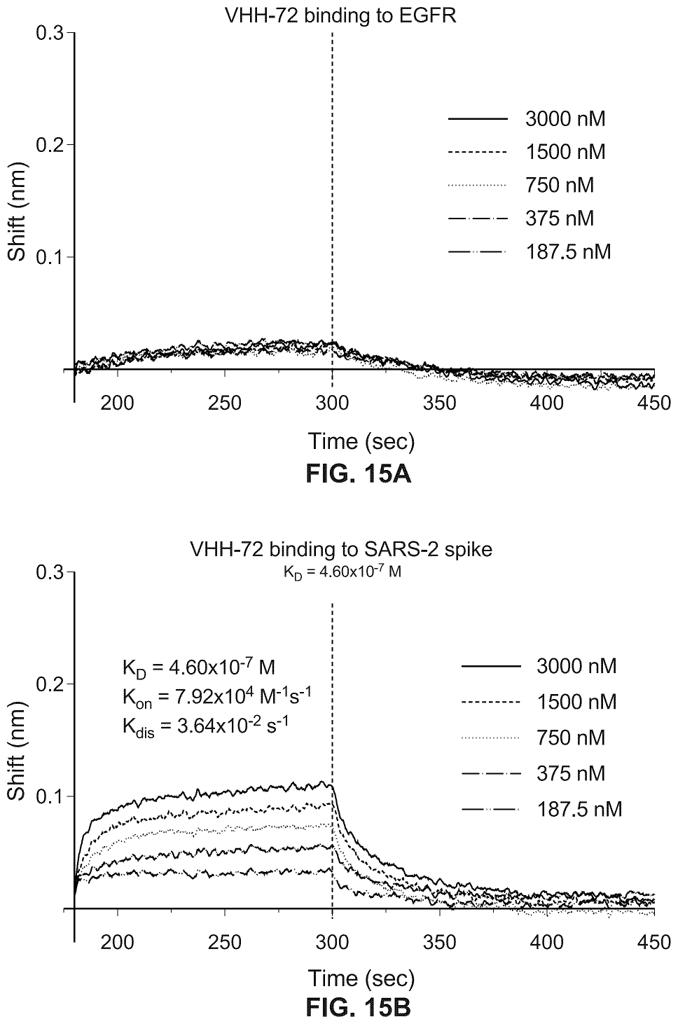

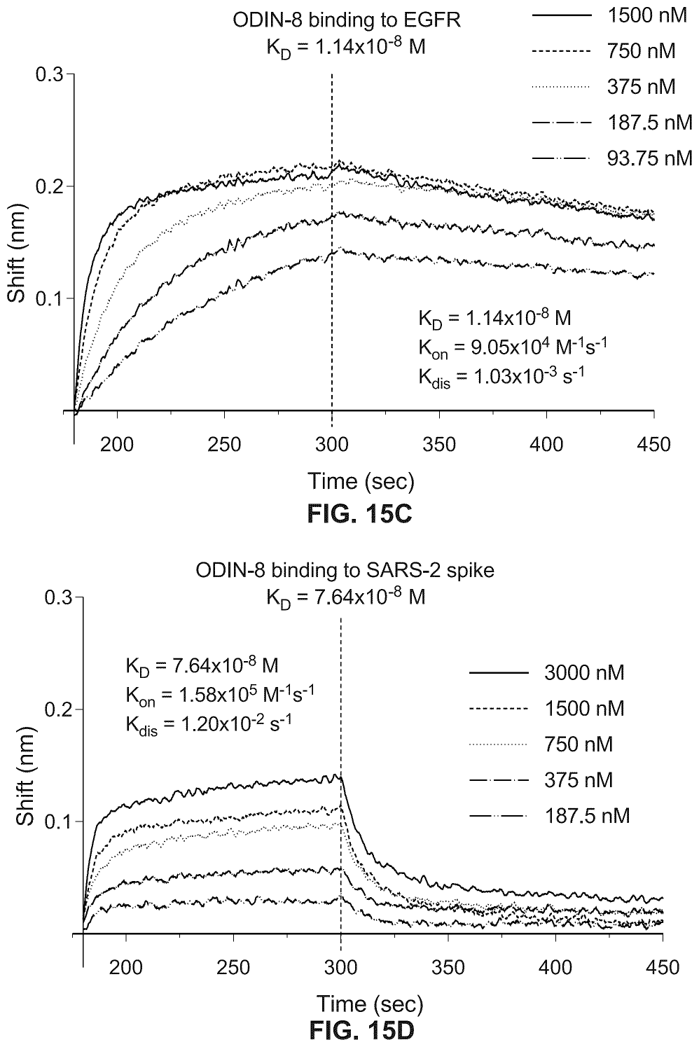

Biolayer interferometry assays demonstrate that VHH-72 does not bind EGFR as shown in FIG. 15A and has affinity for the SARS-CoV-2 spike as expected and shown in FIG. 15B. The ODIN-8 under the same BLI conditions demonstrated a high affinity for both EGFR (See FIG. 15C) and the SARS-CoV-2 spike protein (FIG. 15D).

The ODIN-7 demonstrated a continued ability to neutralize MERS-CoV as shown in FIG. 11B. These data show that post insertion of a different picobody into two different VHHs within the claimed C-C′ loop site does not disrupt the known functionality of either the VHH or picobody paratopes.

### Example 6

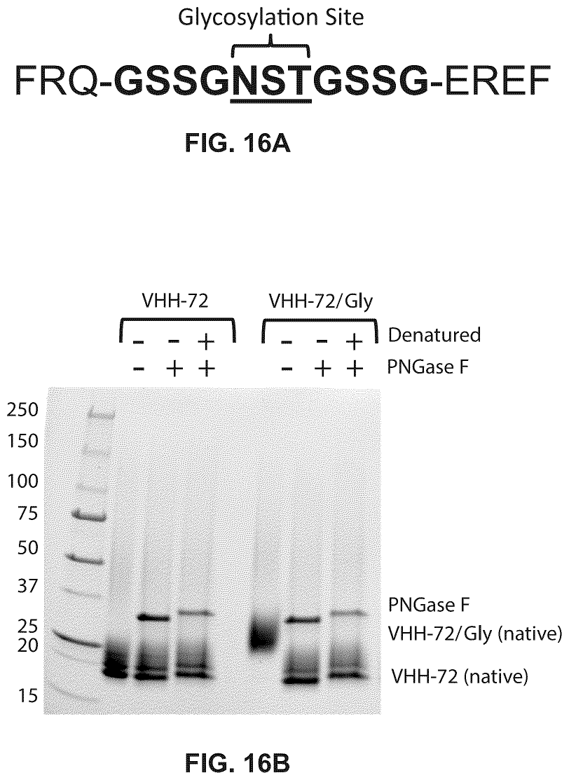

To further illustrate the broad range of variable peptide sequences and/or sequences that could be posttranslationally modified that can be inserted into the ODIN site, the insertion of a glycosylation site sequence was performed and evaluated. An eleven amino acid sequence containing a putative glycosylation site (GSSGNSTGSSG) was inserted into VHH-72 as shown in FIG. 16A. The resulting molecule, VHH-72/Gly, was expressed and purified from human 293-Freestyle cell suspension cultures and purified from cell supernatants by nickel-chelation chromatography. The purified VHH-72/Gly was then shown to have been successfully glycosylated by SDS-PAGE analyses, further the glycan could be removed upon exposure to PNGase F (See FIG. 16B). These data indicate that posttranslational modifications, insertion of unnatural amino acids to be modified or glycan click chemistry is possible at this site.

From the description herein, it will be appreciated that the present disclosure encompasses multiple implementations of the technology which include, but are not limited to, the following:

An FR2 region of a VH, VL, or VHH domain comprising a heterologous polypeptide, wherein the FR2 region comprises a deletion of at least 0, 1, or 2 amino acids.

The FR2 region of any preceding or following implementation, wherein the FR2 region comprises a deletion of no more than 10, 8, 6, or 5 amino acids.

The FR2 region of any preceding or following implementation, wherein the FR2 region comprises a deletion of at least 5 amino acids and no more than 6 amino acids.

The FR2 region of any preceding or following implementation, wherein the FR2 region comprises a deletion of 5 amino acids, optionally wherein the deletion comprises the amino acids 45-49 of the FR2 region (IMGT numbering).

The FR2 region of any preceding or following implementation, wherein the FR2 region comprises a deletion of 6 amino acids, optionally wherein the deletion comprises the amino acids 44-49 of the FR2 region (IMGT numbering).

The FR2 region of any preceding or following implementation, wherein the deletion of the amino acid(s) is within the amino acids 44-49 of the FR2 region (IMGT numbering).

The FR2 region of any preceding or following implementation, wherein the heterologous polypeptide is inserted immediately C-terminal to the amino acid 43, 44, 45, 46, 47, 48, or 49 of the FR2 region (IMGT numbering), optionally wherein the heterologous polypeptide is inserted immediately C-terminal to the amino acid 43 or 44.

The FR2 region of any preceding or following implementation, wherein the FR2 region is of a human, a camelid, or a humanized VH, VL, or VHH domain.

An FW2/HV2 region of a VNAR domain comprising a heterologous polypeptide, wherein the FR2 region comprises a deletion of at least 0, 1, or 2 amino acids.

The FW2/HV2 region of any preceding or following implementation, wherein the FW2/HV2 region comprises a deletion of no more than 14, 12, 10, 8, or 6 amino acids.

The FW2/HV2 region of any preceding or following implementation, wherein the FW2/HV2 region comprises a deletion of at least 5 amino acids and no more than 14 amino acids.

The FW2/HV2 region of any preceding or following implementation, wherein the FW2/HV2 region comprises a deletion of 14 amino acids, optionally wherein the deletion comprises the amino acids 3-16 of the FW2/HV2 region.

The FW2/HV2 region of any preceding or following implementation, wherein the deletion of the amino acid(s) is within the amino acids 3-16 of the FW2/HV2 region.

The FW2/HV2 region of any preceding or following implementation, wherein the heterologous polypeptide is inserted immediately C-terminal to the amino acid 2, 3, 4, 5, 6, 7, 8, 9, 10, 11, 12, 13, 14, or 15 of the FW2/HV2 region.

The FW2/HV2 region of any preceding or following implementation, wherein the FW2/HV2 region is of a shark or a humanized VNAR.

The FR2 region or the FW2/HV2 region of any preceding or following implementation, wherein the heterologous polypeptide comprises one or more amino acids.

The FR2 region or FW2/HV2 region of any preceding or following implementation, wherein the heterologous polypeptide comprises at least 2 amino acids and no more than 50 amino acids.

The FR2 region or FW2/HV2 region of any preceding or following implementation, wherein the heterologous polypeptide comprises at least 51 amino acids.

The FR2 region or FW2/HV2 region of any preceding or following implementation, wherein the heterologous polypeptide comprises an oligomerization domain.

The FR2 region or FW2/HV2 region of any preceding or following implementation, wherein the oligomerization domain is selected from GCN4 leucine zipper, phage T4 fibritin foldon domain, Comp48, and engineered oligomeric beta sheet.

The FR2 region or FW2/HV2 region of any preceding or following implementation, wherein the heterologous polypeptide comprises a cytokine or a chemokine, optionally wherein the cytokine or the chemokine comprises IL-2 or IL-10.

The FR2 region or FW2/HV2 region of any preceding or following implementation, wherein the heterologous polypeptide comprises PD-1, PD-L1, CTLA-4, B7, or CD3.

The FR2 region or FW2/HV2 region of any preceding or following implementation, wherein the heterologous polypeptide comprises a detectable marker, optionally wherein the detectable marker is a GF P or a derivative thereof, or a peptide tag (e.g., a histidine tag (e.g., 8×HIS), a hemagglutinin tag (HA tag; amino acid sequence YPYDVPDYA), a flag tag (amino acid sequence DYKDDDDK), a myc tag (amino acid sequence EQKLISEEDL), a strep tag (WSHPQFEK), and/or an A56R protein).

The FR2 region or FW2/HV2 region of any preceding or following implementation, wherein the heterologous polypeptide comprises an enzyme, optionally wherein the enzyme is a luciferase.

The FR2 region or FW2/HV2 region of any preceding or following implementation, wherein the heterologous polypeptide comprises a polymer (e.g., polyethylene glycol (P E G)) or a polypeptide that extends the serum half-life.

The FR2 region or FW2/HV2 region of any preceding or following implementation, wherein the polypeptide that extends the serum half-life is selected from an albumin-binding protein, an anti-albumin antibody or a fragment thereof (e.g., CA645), albumin (e.g., human serum albumin), an immunoglobulin, an Fc domain, a fragment of an Fc domain, and an FcRnBP.

The FR2 region or FW2/HV2 region of any preceding or following implementation, wherein the heterologous polypeptide comprises at least one natural or unnatural amino acid.

The FR2 region or FW2/HV2 region of any preceding or following implementation, wherein (a) the at least one natural amino acid comprises a cysteine, cystine, tyrosine, serine, threonine, lysine, and/or histidine; and/or (b) the at least one unnatural amino acid comprises an unnatural amino acid comprising an azide, alkynes, an aldehyde, an aminooxy, a functionalized arene, or a trans-cyclooctene (e.g., for bio-orthogonal labeling); fluorosulfate-L-tyrosine (FSY); L-Azidohomoalanine hydrochloride; L-Azidonorleucine hydrochloride; p-acetylphenylalanine (pAcPhe); para-acetylphenylalanine (pAF); para-azidophenylalanine (pAZ); N6-((2-azidoethoxy)carbonyl)-I-lysine; a cysteine and selenocysteine derivative; a leucine derivative; a phenylalanine derivative; a lysine derivative; a tryptophan derivative; and/or a tyrosine derivative.

The FR2 region or FW2/HV2 region of any preceding or following implementation, wherein the heterologous polypeptide: (a) comprises a glycosylation site, wherein the glycosylation site comprises an amino acid sequence of NXT or NXS, wherein X is any amino acid except proline; and/or (b) is glycosylated and comprises a glycan.

The FR2 region or FW2/HV2 region of any preceding or following implementation, wherein the least one natural or unnatural amino acid, or the glycan is conjugated.

The FR2 region or FW2/HV2 region of any preceding or following implementation, wherein the at least one natural or unnatural amino acid, or the glycan is conjugated to a polyethylene glycol (PEG), a chemotherapeutic agent, and/or a cytotoxic agent.

The FR2 region or FW2/HV2 region of any preceding or following implementation, wherein the heterologous polypeptide comprises a linker, optionally wherein the linker comprises the amino acid sequence of GSSG or GSSGSSG.

The FR2 region or FW2/HV2 region of any preceding or following implementation, wherein the heterologous polypeptide comprises an antigen-binding protein.

The FR2 region or FW2/HV2 region of any preceding or following implementation, wherein the antigen-binding protein comprises any one of the antigen-binding proteins listed in Tables 7-10, or a fragment thereof.

The antigen-binding protein of any preceding or following implementation, wherein the antigen-binding protein binds: (a) an antigen expressed on a virus, a cancer cell, a neuron, a motor neuron, and/or an immune cell; or (b) a cytokine or a toxin.

The FR2 region or FW2/HV2 region of any preceding or following implementation, wherein the antigen-binding protein comprises an ultralong CDR3 (UL-CDR3), scFV, Fab′, F(ab′)2, ds-scFv, scFab′, diabody, scFV-CH3 (minibody), a VHH domain, a VH domain, a VL domain, or a VNAR domain.

A VH domain comprising the FR2 region of any preceding or following implementation.

A VL domain comprising the FR2 region of any preceding or following implementation.

A VHH domain comprising the FR2 region of any preceding or following implementation.

A VNAR domain comprising the FW2/HV2 region of any preceding or following implementation.

An antigen-binding protein comprising the FR2 region or FW2/HV2 region of any preceding or following implementation, the VH domain of any preceding or following implementation, the VL domain of any preceding or following implementation, the VHH domain of any preceding or following implementation, and/or the VNAR domain of any preceding or following implementation.

The antigen-binding protein of any preceding or following implementation, wherein the antigen-binding protein comprises an Fc domain.

The antigen-binding protein of any preceding or following implementation, wherein the antigen-binding protein comprises at least two of the FR2 region and/or FW2/HV2 region.

The antigen-binding protein of any preceding or following implementation, wherein the antigen-binding protein comprises (a) any two of the FR2 region and/or FW2/HV2 region and (b) an Fc domain, wherein the any two of the FR2 region and/or FW2/HV2 region are fused to the N-terminus of the Fc domain.

The antigen-binding protein of any preceding or following implementation, wherein the antigen-binding protein comprises at least four of the FR2 region and/or FW2/HV2 region.

The antigen-binding protein of any preceding or following implementation, wherein the antigen-binding protein comprises (a) any four of the FR2 region and/or FW2/HV2 region and (b) an Fc domain, wherein the two of the FR2 region and/or FW2/HV2 region are fused to the N-terminus of the Fc domain, and the other two of the FR2 region and/or FW2/HV2 region are fused to the C-terminus of the Fc domain.

The antigen-binding protein of any preceding or following implementation, further comprising a detectable marker or a peptide tag.

The antigen-binding protein of any preceding or following implementation, wherein the detectable marker or the peptide tag is selected from a GF P or a derivative thereof, a histidine tag (e.g., 8×HIS), a hemagglutinin tag (HA tag; amino acid sequence YPYDVPDYA), a flag tag (amino acid sequence DYKDDDDK), a myc tag (amino acid sequence EQKLISEEDL), a strep tag (WSHPQFEK), and an A56R protein.

The antigen-binding protein of any preceding or following implementation, further comprising an enzyme, optionally wherein the enzyme is a luciferase.

The antigen-binding protein of any preceding or following implementation, further comprising a polymer (e.g., polyethylene glycol (PEG)) or a polypeptide that extends the serum half-life.

The antigen-binding protein of any preceding or following implementation, wherein the polypeptide that extends the serum half-life is selected from an albumin-binding protein, an anti-albumin antibody or a fragment thereof (e.g., CA645), albumin (e.g., human serum albumin), an immunoglobulin, an Fc domain, a fragment of an Fc domain, and an FcRnBP.

The antigen-binding protein of any preceding or following implementation, wherein the antigen-binding protein comprises any one of the antigen-binding proteins listed in Tables 7-10, or a fragment thereof.

The antigen-binding protein of any preceding or following implementation, wherein the antigen-binding protein binds: (a) an antigen expressed on a virus, a cancer cell, a neuron, a motor neuron, and/or an immune cell; or (b) a cytokine or a toxin.

A chimeric antigen receptor comprising the FR2 region or FW2/HV2 region of any preceding or following implementation, the VHH domain of any preceding or following implementation, the VL domain of any preceding or following implementation, the VHH domain of any preceding or following implementation, the VNAR domain of any preceding or following implementation, and/or the antigen-binding protein of any preceding or following implementation.

The chimeric antigen receptor of any preceding or following implementation, wherein the chimeric antigen receptor binds at least two antigens (e.g., dual CAR).

An isolated nucleic acid that encodes the FR2 region or FW2/HV2 region of any preceding or following implementation, the VH domain of any preceding or following implementation, the VL domain of any preceding or following implementation, the VHH domain of any preceding or following implementation, the VNAR domain of any preceding or following implementation, the antigen-binding protein of any preceding or following implementation, and/or the chimeric antigen receptor of any preceding or following implementation.

A vector comprising the nucleic acid of any preceding or following implementation.

A cell comprising the FR2 region or FW2/HV2 region of any preceding or following implementation, the VH domain of any preceding or following implementation, the VL domain of any preceding or following implementation, the VHH domain of any preceding or following implementation, the VNAR domain of any preceding or following implementation, the antigen-binding protein of any preceding or following implementation, the chimeric antigen receptor of any preceding or following implementation, the nucleic acid of any preceding or following implementation, and/or the vector of any preceding or following implementation.

The cell of any preceding or following implementation, wherein the cell is a prokaryotic cell or a eukaryotic cell.

The cell of any preceding or following implementation, wherein the eukaryotic cell is a mammalian cell or a fungus (e.g., yeast, e.g., Pichia pastoris, e.g., for producing the proteins or for yeast display).

The cell of any preceding or following implementation, wherein the prokaryotic cell is a bacterium.

The cell of any preceding or following implementation, wherein the cell is a human cell.

The cell of any preceding or following implementation, wherein the cell is a T cell, an NK cell, or a macrophage (e.g., CAR-T, CAR-NK, CAR-M).

A virus comprising the FR2 region or FW2/HV2 region of any preceding or following implementation, the VH domain of any preceding or following implementation, the VL domain of any preceding or following implementation, the VHH domain of any preceding or following implementation, the VNAR domain of any preceding or following implementation, the antigen-binding protein of any preceding or following implementation, the chimeric antigen receptor of any preceding or following implementation, the nucleic acid of any preceding or following implementation, and/or the vector of any preceding or following implementation.

The virus of any preceding or following implementation, wherein the virus is a bacteriophage (e.g., for phage display), a vaccinia (e.g., for vaccinia display), or an AAV.

A conjugate comprising the FR2 region or FW2/HV2 region of any preceding or following implementation, the VH domain of any preceding or following implementation, the VL domain of any preceding or following implementation, the VHH domain of any preceding or following implementation, the VNAR domain of any preceding or following implementation, the antigen-binding protein of any preceding or following implementation, optionally wherein the conjugate comprises a polyethylene glycol (PEG), a chemotherapeutic agent, and/or a cytotoxic agent.

A pharmaceutical composition comprising the FR2 region or FW2/HV2 region of any preceding or following implementation, the VH domain of any preceding or following implementation, the VL domain of any preceding or following implementation, the VHH domain of any preceding or following implementation, the VNAR domain of any preceding or following implementation, the antigen-binding protein of any preceding or following implementation, the chimeric antigen receptor of any preceding or following implementation, the nucleic acid of any preceding or following implementation, the vector of any preceding or following implementation, the cell of any preceding or following implementation, and/or the conjugate of any preceding or following implementation.

A kit comprising the FR2 region or FW2/HV2 region of any preceding or following implementation, the VH domain of any preceding or following implementation, the VL domain of any preceding or following implementation, the VHH domain of any preceding or following implementation, the VNAR domain of any preceding or following implementation, the antigen-binding protein of any preceding or following implementation, the chimeric antigen receptor of any preceding or following implementation, the nucleic acid of any preceding or following implementation, the vector of any preceding or following implementation, the cell of any preceding or following implementation, the conjugate of any preceding or following implementation, and/or the pharmaceutical composition of any preceding or following implementation.

A method of producing the FR2 region or FW2/HV2 region of any preceding or following implementation, the VH domain of any preceding or following implementation, the VL domain of any preceding or following implementation, the VHH domain of any preceding or following implementation, the VNAR domain of any preceding or following implementation, the antigen-binding protein of any preceding or following implementation, or the chimeric antigen receptor of any preceding or following implementation, the method comprising the steps of: (i) culturing a cell comprising the nucleic acid of any preceding or following implementation or the vector of any preceding or following implementation under conditions suitable to allow expression; and (ii) recovering the expressed FR2 region, FW/HV2 region, VH domain, VL domain, VHH domain, VNAR domain, antigen-binding protein, or chimeric antigen receptor.

The method of any preceding or following implementation, wherein the cell is a prokaryotic cell or a eukaryotic cell.

The cell of any preceding or following implementation, wherein the eukaryotic cell is a mammalian cell or a fungus (e.g., yeast, e.g., Pichia pastoris).

The cell of any preceding or following implementation, wherein the prokaryotic cell is a bacterium.

A method of preventing or treating a disease in a subject, the method comprising administering to the subject the pharmaceutical composition of any preceding or following implementation.

The method of any preceding or following implementation, wherein the disease is selected from a cancer, an inflammatory disease, a neurological disorder, a musculoskeletal disorder, an ophthalmology disease, a genetic disease, a hematological disorder, high cholesterol, and an infection.

The method of any preceding or following implementation, wherein the infection is a viral infection, a bacterial infection, or a fungal infection.

The method of any preceding or following implementation, wherein the infection is a viral infection, optionally wherein the viral infection is a SARS-CoV-2 and/or an HIV infection (e.g., HIV-1).

The method of any preceding or following implementation, wherein the disease is a cancer, optionally wherein a cancer is a solid tumor.

The method of any preceding or following implementation, further comprising conjointly administering to the subject an additional cancer therapy.

The method of any preceding or following implementation, wherein the additional cancer therapy is selected from the group consisting of immunotherapy, checkpoint blockade, cancer vaccines, chimeric antigen receptors, chemotherapy, radiation, target therapy, and surgery, optionally wherein the additional cancer therapy is checkpoint blockade.

The method of any preceding or following implementation, wherein the subject is a mammal, optionally a mouse, a dog, a cat, or a human.

As used herein, the term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C,” within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment,” “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to 5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to 5°, less than or equal to 4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed. Accordingly, the reader should consult the application as filed for the original content of the disclosure. Any deletion of content of the disclosure should not be construed as a disclaimer, forfeiture or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

TABLE 1
VHH-based Molecules in Clinical Trials

| | | Nanobody's | | Current |
|---|---|---|---|---|
| Drug Name(s) | Format | Target(s) | Indication | Status |
| Caplacizumab | Bivalent, | Von Willebrand | Acquired thrombotic | FDA/EMA |
| ALX-0081, | monospecific | factor-A | thrombocytopenia | approved |
| ALX-0681 | | | purpura | |
| KN046 | Tetravalent, | CTLA-4, PD-L1 | Advanced solid tumors | Phase II/III |
| | bispecific, Fc- | | and lymphoma | |
| | fusion protein | | | |
| Ozoralizumab | Trivalent, | TNFα (2), HSA | Rheumatoid arthritis | Phase II |
| ATN-103 | bispecific | | | |
| Vobarilizumab | Bivalent, | IL-6R, HSA | Rheumatoid arthritis, | Phase II |
| ALX-0061 | bispecific | | systemic lupus | |
| | | | erythematosus | |
| Sonelokimab | Trivalent, | IL-17F, IL-17A/F, | Psoriasis | Phase II |
| M1095 | bispecific | HSA | | |
| Lulizumab | Monomeric, | CD28 | Systemic lupus | Phase II |
| BMS931699 | pegylated | | erythematosus, | |
| | | | kidney transplantation | |
| ALX-0171 | Trivalent, | RSV F-protein | RSV lower respiratory | Phase II |
| | monospecific | | tract infection | |
| LMN-101 | Monomeric | FLaA flagellin of | C. jejuni infection | Phase II |
| | | Campylobacter jejuni | | |
| ARP1 | Monomeric | Rotavirus | Rotavirus infection | Phase II |
| VHH batch | | | | |
| 203027 | | | | |
| Envolimab | Monospecific, | PD-L1 | Advanced solid | Phase II |
| KN035 | Fc-fusion | | tumors, multiple | |
| | protein | | primary neoplasm | |
| INBRX-109 | Tetravalent, | Death receptor 5 | Advanced solid | Phase I/II |
| | monospecific, | | tumors, conventional | |
| | Fc-fusion | | chondrosarcoma | |
| | protein | | | |
| KN044 | Monospecific, | CTLA-4 | Advanced solid tumors | Phase I |
| | Fc-fusion | | | |
| | protein | | | |
| ALX-0141 | Trivalent, | RANKL (2), HSA | Osteoporosis and | Phase I |
| | bispecific | | bone metastasis | |
| M6495 | Bivalent, | ADAMTS-5, HSA | Osteoarthritis | Phase I |
| | bispecific | | | |
| ES101 | Tetravalent, | PD-L1, CD137 | Advanced solid tumors | Phase I |
| INBRX-105 | bispecific, Fc- | | | |
| | fusion protein | | | |
| ES102 | Hexavalent, | OX40 | Advanced solid tumors | Phase I |
| INBRX-106 | monospecific, | | | |
| | Fc-fusion | | | |
| | protein | | | |
| BCMA | Nanobody- | BCMA | Relapsed/Refractory | Phase I |
| nanobody | based chimeric | | Myeloma | |
| CAR-T cells | antigen receptor | | | |
| CD19/CD20 | Nanobody- | CD19/CD20 | B-Cell Lymphoma | Phase I |
| bispecific | based bispecific | | | |
| CAR-T cells | chimeric antigen | | | |
| | receptor | | | |
| αPD1-MSLN- | MSLN-CAR T | PD-1 | Advanced solid tumors | Phase I |
| CAR T cells | cells secreting | | | |
| | anti-PD-1 | | | |
| | nanobody | | | |
| ALX-0651 | Biparatopic, | CXCR4 | Multiple myeloma, | Phase I |
| | monospecific | | non-Hodgkin's lymphoma | terminated |
| TAS266 | Tetrameric | Death receptor 5 | Advanced solid tumors | Phase I |
| | | | | terminated |

TABLE 2
Definition of the FR-IMGT and CDR-IMGT
AA Number

| | | | | | | | |
|---|---|---|---|---|---|---|---|
| 1→26 | | 39→55 | | 66→104 | | | |
| (C23) | 27→38 | (W 41) | 56→65 | (C-104) | 105→116 | 105→117 | 118→129 |

Number of AA's

| | | | | | | 2-13 or | |
|---|---|---|---|---|---|---|---|
| 25-25 | 5-12 | 16-17 | 0-10 | 36-39 | 2-12 | more | 10-12 |
| FRI- | CDRI- | FR2- | CDR2- | FR3- | Specific | Specific to | |
| IMGT | IMGT | IMGT | IMGT | IMGT | to V | V-Domain | |
| | | | | | Region | | |
| | | | | | Germline | Rearranged | FR4- |
| | | | | | CDR3-IMGT | CDR3-IMGT | IMGT |

TABLE 3
Representative FR2 sequences of VHH structures
in the Protein Data Bank (PDB)

| PDB | | PDB | |
|---|---|---|---|
| ID | FR2 | ID | FR2 |
| 1KXQ | VGWFRQAPGKEREGVAA | 10P9 | SGWFRQAPGKEREGVAA |
| 1I3U | IGWFRQAPGKERIYLAT | 6ANA | LHWIKQRPGQGLEWIGY |
| 4KRN | MGWFRQAPGKQREFVAA | 5TOK | IGWFRQAPGKEREGVSC |
| 7LVU | TGWFRQAPGKEREFVAL | 5TOJ | IGWFRQAPGKEREAVSC |
| 1KXV | MSWYRRAPGKGRDFVSG | 5KTZ | MGWFRQAPGKENEGVAA |
| 3QXW | MAWFRQAPGKEREFVAK | 2P43 | MGWFRQAPGKEREGVAA |
| 3K3Q | MGWFRQAPGKDREFVAA | 3STB | MGWFRQAPGKEREFVAA |
| 4POU | MGWFRQAPGKEREGVAA | 4NC2 | MGWFRQAPGKEREFVAA |
| 1G9E | MGWFRQAPGKERESVAA | 4P2C | MGWYRQAPGKQREFVAS |
| 1YC7 | TGWYRQAPGKEREWVSS | 4YGA | VGWFRQVPGKEREKLLC |
| 6S0Y | IGWFRQAPGKGRQGVSC | 5KU0 | MGWFRQAPGKENEGVAA |
| 1QD0 | MGWFRQVPGKEREFVAA | 5KWL | MGWFRQAPGKENEGVAA |
| 6F0D | MGWFRQAPGKEREFVAA | 5VM4 | VGWFRQIPGKEREFVGG |
| 6EZW | MGWFRQAPGKEREFVAA | 6UL4 | VGWFRQAPGKEREGVSC |
| 3K81 | WGWYRRAPGKQRELIAA | 6X03 | MGWFRQAPGKEREFVAA |
| 4NC1 | MAWFRQPPGKEREFVAA | 6X03 | MGWFRQAPGQEREFVAR |
| 4NC1 | VAWFRQAPGAEREFVAV | 3R0M | MGWFRQAPGKEREFVAA |
| 5U65 | MRWFRQAPGKGREGVAS | 2XXC | MAWYRQAPGKARELIAA |
| 5U64 | IGWFRQAPGKEREGVAV | 1ZVY | MTWFRQAPGKAREGVAA |
| 1BZQ | MGWFRQAPGKEREGVAA | 6O25 | IHWVRQAPGKGLEWVAV |
| 5UKB | LGWFRQAPGKEREFVAA | 4WEU | MGWFRQAPGKKREYVAA |
| 5UK4 | MGWFRQAPGKERDFVAK | 4WEM | MGWFRQAPGKQREFVGY |
| 5TJW | QGWFRQPPGKEREFVAS | 4WEN | MGWYRQAPGSKREFVAH |
| 6DYX | MGWFRQTPGKERELVAA | 5O05 | MGWYRQTPGNQRELVAT |
| 6RBB | MGWLRQAPGKGTEFVAA | 4NBX | MAWFRQPPGKEREFVAA |
| 6DBE | MGWFRQAPGKEREFVAD | 5OMN | MGWYRQAPGKERELVAS |
| 6DBD | VGWYRQGPGKQRDVVAS | 5H8D | MSWYRQAPGKERESVAR |
| 6DBA | MGWFRQVPGKEREGVAR | 5TP3 | IGWFRQAPGKEREAVSC |
| 4B50 | MSWYRQAPGKQRELVAF | 5O03 | IGWFRQAPGKGLEGVSC |
| 5O04 | VGWYRQAPEKQRELVAQ | 5O02 | MRWYRQAPGKERELVAA |
| 5O04 | MGWYRQAPGKQRELVAS | 5OMM | VGWYRQAPGNQRELVAS |
| 6ITQ | WAWFRQGPGTEREGVAA | 4LGP | IGWFRQAPGKEHEWLVC |
| 1ZVH | LGWFRQAPGKEREGVAA | 6OBE | MGWFRQAPGRERELVAV |
| 5L21 | VNWYRQPPGKSREFDEL | 6OBG | MGWYRQAPGKERELVAD |
| 3RJQ | MGWFRQAPGKEREFVAA | 6OCD | IGWFRQAPGKEYGGVSC |
| 6WAQ | MGWFRQAPGKEREFVAT | 6OCA | LGWFRQAPGKEREFVGS |
| 1ZV5 | IGWFRQAPGKDREGVVF | 6OBM | MGWYRQAPGKERELVAD |
| 6CWG | LAWFRQAPGKEREFVAA | 5BOZ | MGWFRQAPGKEREFVAS |
| 5HM1 | AGWFRQAPGKQREPVAT | 1JTP | MGWFRQAPGKEREGVAA |
| 6WAR | MDWYRQAPGKQRELVAG | 6X07 | MGWYRQAPGKERELVAS |
| 4KRL | MGWFRQAPGKEREFVSG | 5U4M | IGWFRQAPGKEREGLSS |
| 5VLV | MAWFRQAPGKEREFVAG | 2XV6 | MAWYRQAPGKARELIAA |
| 5VM6 | VGWFRQIPGKEREFVGG | 6H16 | IGWFRQAPGKEREFVAE |
| 4NC0 | VAWFRQAPGAEREFVAV | 1MVF | MGWFRQAPGKEREGVAA |
| 4NBY | MAWFRQPPGKEREFVAA | 1U0Q | VGWFRQAPGKEREFVGY |
| 7KN5 | IGWFRQAPGKEREGVSC | 2BSE | MGWFRQLAGKEPELVVA |
| 7KN5 | IGWFRQAPGKEREGVSC | 4IDL | MGWYRRAPGKQREWVAS |
| 2P49 | MGWFRQAPGKEREGVAA | 5HVH | MGWYRQAPGKQRELVAA |
| 2P4A | MGWFRQAPGKEREGVAA | 5HVH | MGWYRQAPGKERELVAA |
| 7K84 | MGWFRQGPGKEREFVAT | 5KU2 | MGWFRQAPGKELEGVAG |
| 7K7Y | MGWFRQAPGKEREYVAA | 6H15 | IGWFRQAPGKEREFVAA |
| 5O2U | MGWWRQAPGKEREFVAR | 6ZRV | IGWFRQAPGKEREGVSC |
| 4HEM | MGWFRQAPGPEREFVAT | 3JBC | MGWFRQAPGKEREGVAG |
| 4KRM | MGWFRQAPGKEREFVSG | 3JBD | MAWFRQAPEKEREWVAC |
| 4KRO | MGWFRQAPGKQREFVAA | 3JBE | MAWFRQVLGEGREGVAF |
| 4KRO | VHWVRQSPGKGLEWLGV | 3JBF | MGWYRQVRGKEREGVAF |
| 4KRP | MGWFRQAPGKEREFVVA | 7KN6 | MGWARQVPGKGLEWVSY |
| 5JMR | MYWVRQAPGKGLEWVSS | 7C8W | MEWYRQAPGKEREGVAA |
| 5M2J | MYWVRQAPGKGLEWVSE | 7CAN | MEWYRQAPGKEREGVAA |
| 5M2M | IGWFRQAPGKEREFVAR | 7D2Z | MAWYRQAPGKEREWVAA |
| 5M2I | MGWFRQAPGKERELLGN | 7D30 | MEWYRQAPGKEREGVAA |
| 5VL2 | MGWFRQAPGKEREFVGG | 7D30 | MAWYRQAPGKEREWVAA |
| 1SJX | MGWYRQAPGTQRELVAA | 4GRW | IAWFRQAPGKEREGVSG |
| 6KSN | MGWFRQAPGKEREGVAF | 5J1T | MGWYRQAPGKQRELVAA |
| 6CWK | LAWFRQAPGKEREFVAA | 1XFP | MGWFRQAPGKEREGVAA |
| 5M30 | MAWYRQAPGKQRELVAS | 6Z43 | MGWFRQAPGKEREFVAA |
| 6JB9 | MTWFRQAPGKAREGVAA | 7KKK | MGWYRQAPGKERELVAG |
| 1F2X | MGWFRQAPGKEREGVAT | 7KKL | MGWYRQAPGKERELVAG |
| 6H6Y | SGWFRQAPGKEREFLAS | 3DWT | LGWFRQAPGQEREAVAA |
| 6H6Z | MAWFRQAPGKEREFVSG | 6APP | VAWYRQVPGKGRELLSS |
| 6H71 | MGWYRQAPGKERELVAT | 1RI8 | MGWFRRAPGKEREGVAV |
| 6UC6 | IAWYRQAPGKEREGVSC | 4QGY | IGWFRQAPGKEREGVSC |
| 6UFT | VGWFRQAPGKEREGVSC | 4W2P | MGWYRRAPGKQRELVAS |
| 6UHT | IGWVRQAPGKEREGVSC | 6APO | VAWYRQVPGKGRELLSS |
| 6UI1 | MGWYRQAPGKRREMVAD | 6APQ | LGWYRRAPGKEREFVAR |
| 6U11 | MGWYRQVPGKPRELVAD | 5LWF | MGWFRKAPGKEREFVAA |
| 6UI1 | VGWYRQAPGKQREMVAS | 5E7B | MGWYHQAPGNECELVSA |
| 6UL6 | MGWYRQVPGKPRELVAD | 7KKJ | MGWYRQAPGKERELVAG |
| 6UL6 | VGWYRQAPGKQREMVAS | 5HVF | MGWYRQAPGKERELVAA |
| 6UL6 | MGWYRQAPGKRREMVAD | 5HVG | MGWYRQAPGKQRELVAA |
| 4HEP | IGWFRQAPGKEREGVSY | 4GRW | IGWFRQAPGKEREGVSC |
| 1G6V | MGWFRQAPGKEREGVAT | 4GRW | VGWFRQAPGKEREFVAA |
| 5E1H | MAWFRQAPGKEREFVAG | 5FUC | MGWFRQAPGKEREGVAH |
| 3JBG | MRWFRQAP-KEREWVAS | 6XW4 | MGWFRQAPGKEREFVAA |
| 6OBO | MGWYRQAPGKERELVAD | 6XW6 | MGWFRQAPGKEREFVAA |
| 6X05 | IGWFRQRRGEQREEIAY | 6XW5 | MGWFRQAPGEDRAFVTS |
| 6X06 | MGWFRQAPGKEREFVAA | 4X7F | MAWYRQGPGEQRELVAV |
| 4LHJ | MGWYRQAPGKERELVAT | 3EZJ | MDWDRQAPGKQRELVAT |
| 4LHQ | MGWFRQAPGKEREFVAA | 3SN6 | MNWVRQAPGKGLEWVSD |
| 6OBC | MGWYRQAPGKPRELVAE | 6U54 | MAWYRQAPGKERDWVGT |
| 6QTL | MAWFRQAPGKEREFLAT | 6GWN | IAWFRQAPGKEREGVSC |
| 6QTL | MAWFRQAPGKEREFLAT | 7C8V | MWWYRQAPGKEREWVAA |
| 1MEL | MGWFRQAPGKEREGVAA | 7KN6 | MSWVRQAPGKGLEWVSV |
| 2XT1 | MAWYRQAPGKARELIAA | 4B5E | MGWFRQAPGKERDVVAA |
| 6OQ7 | VTWSRQAPGKSLQWVAS | 1RJC | MGWFRQAPGKEREGVAV |
| 6OQ6 | IGWFRQPPGKEREAVSY | 3OGO | MRWYRQAPGKEREWVAG |
| 6OQ8 | MAWYRQPPGKQREWVAS | 5NQW | MAWVRQAPGKRPEWVSS |
| 4TVS | VGWFRQAPGLEREFVAT | 5VXL | MGWYRQAPGKQRELVAS |
| 6V7Y | MGWFRQAPGKEREIVAG | 5VXK | IAWFRQAPGKEREGVSC |
| 1ZA3 | IHWVRQAPGKGLEWVAS | 5VXM | IGWFRQAPGKEREGVSC |
| 6V80 | MGWYRQAPGKQRELVAT | 1IEH | MAWVRQAPGKGLEWVST |
| 6V7Z | MGWYRQAPGKQRDFLAV | 5VXJ | MNWYRQAPGKERELVAR |
| 4LAJ | IGWFRQAPGKEREGVSC | 6GWN | MQWYRQSPGTERELVAA |
| 5H8O | MSWYRQAPGKERESVAR | 4XC | MGWYRQAPGKQRELVAS |
| 6OYA | MNWVRQAPGKGLQWVSD | 6OQ5 | IGWFRQPPGKEREAVSY |
| 5JMO | MYWVRQAPGKGLEWVSS | 6OQ5 | VTWSRQAPGKSLQWVAS |
| 6YU8 | MAWFRQAPGKEREFVVA | 6OQ5 | MAWYRQPPGKQREWVAS |
| 6OC8 | VGYYRQTPGRQRELIAG | 6HEQ | MGWFRQAPGKGREFVAS |
| 1HCV | MGWFRQAPGKERESVAA | 6U51 | MGWYRRAPGNERELVAG |
| 6JB8 | MTWFRQAPGKAREGVAA | 6U50 | MGWYRRAPGNERELVAG |
| 7A6O | MGWFRQAPGKGRELVAA | 6U53 | MAWYRQAPGKERDWVGT |
| 3TPK | VIWFRQAPGKEREIVSG | 6U52 | MGWYRRAPGNERELVAG |
| 2XA3 | MGWFRQAPGKEREFVAA | 6U55 | VGWFRQAPGKERELVAL |

TABLE 4
FR2 Sequences in IGHV (heavy chain) From Representative Species

| | |
|---|---|
| BOVINE | |
| Bos_taurus_IGHV1-10*01 | VGWVRQAPGKALECLGG |
| Bos_taurus_IGHV1-14*01 | VGWVRQAPGKALEWLGV |
| Bos_taurus_IGHV1-17*01 | VSWVRQAPGKALEWLGD |
| Bos_taurus_IGHV1-21*01 | VGWVRQAPGKALEWVGG |
| Bos_taurus_IGHV1-25*01 | IHWVRQAPGKALEWLGD |
| Bos_taurus_IGHV1-30*01 | VVWVRQAPGKALEWLGG |
| Bos_taurus_IGHV1-39*01 | VSWVRQAPGKALEWLGG |
| Bos_taurus_IGHV1-7*01 | VGWVRQAPGKALEWLGG |
| DOG | |
| Canis_lupus_familiaris_IGHV1-30*01 | MIWVRQAPGAGLDWMGQ |
| Canis_lupus_familiaris_IGHV3-10*01 | MDWVYQAPGKGLQWVTY |
| Canis_lupus_familiaris_IGHV3-16*01 | MFWIRQAPGKGNQWVGY |
| Canis_lupus_familiaris_IGHV3-18*01 | MSWVRQAPGKGLQWVAY |
| Canis_lupus_familiaris_IGHV3-19*01 | MSWVRQAPEKGLQLVAG |
| Canis_lupus_familiaris_IGHV3-2*01 | MSWIRQAPGKGLQWVSQ |
| Canis_lupus_familiaris_IGHV3-23*01 | MSWVRQAPGKGLQGVSL |
| Canis_lupus_familiaris_IGHV3-24*01 | MSWFCQAPRKGLQWVAE |
| Canis_lupus_familiaris_IGHV3-3*01 | MYWARQAPGKGLQWVSH |
| Canis_lupus_familiaris_IGHV3-35*01 | MNWVRQAPGKGLQWVAY |
| Canis_lupus_familiaris_IGHV3-37*01 | MSWVHQAPGQGLQWVSW |
| Canis_lupus_familiaris_IGHV3-38*01 | MSWVRQSPGKGLQWVAV |
| Canis_lupus_familiaris_IGHV3-39*01 | MSWIRQAPGKGLQWVAD |
| Canis_lupus_familiaris_IGHV3-41*01 | MSWVRQAPGKGLQWVAA |
| Canis_lupus_familiaris_IGHV3-47*01 | MSWVRQAPGKGLQWVTA |
| Canis_lupus_familiaris_IGHV3-5*01 | MSWVRQAPGKGLQWVAY |
| Canis_lupus_familiaris_IGHV3-50*01 | MDWVRQAPGKGLQWLTR |
| Canis_lupus_familiaris_IGHV3-54*01 | MNWVRQAPGKGLQWVGY |
| Canis_lupus_familiaris_IGHV3-58*01 | KSWVRQAPGKGLKWVAV |
| Canis_lupus_familiaris_IGHV3-6*01 | MSWIRQAPGKGLQWVAY |
| Canis_lupus_familiaris_IGHV3-61*01 | IYWVHEAPGKGLQWVAA |
| Canis_lupus_familiaris_IGHV3-67*01 | MYWVRQAPGKGLQWVAR |
| Canis_lupus_familiaris_IGHV3-7*01 | MSWVRQAQKKGLQWVGH |
| Canis_lupus_familiaris_IGHV3-70*01 | MSWFCQAPGKGLQCVAE |
| Canis_lupus_familiaris_IGHV3-75*01 | MSWVCQAPGKGLQWVAA |
| Canis_lupus_familiaris_IGHV3-76*01 | ISWVRQAPGKGLHWVSV |
| Canis_lupus_familiaris_IGHV3-8*01 | MYWVRQAPGKGLEWVAR |
| Canis_lupus_familiaris_IGHV3-80*01 | MEWVRQAPGKGLQWVAQ |
| Canis_lupus_familiaris_IGHV3-81*01 | MDWVRQAPGKRLQWVAG |
| Canis_lupus_familiaris_IGHV3-82*01 | MHWVRQATGKGLQWVTR |
| Canis_lupus_familiaris_IGHV3-9*01 | MDWVRQAPGKGLQWLSE |
| Canis_lupus_familiaris_IGHV4-1*01 | WNWIRQRPGRGLEWMGY |
| ZEBRAFISH | |
| Danio_rerio_IGHV1-1*01 | MHWIRQKPGKGLEWIGY |
| Danio_rerio_IGHV1-2*01 | MHWIRQKPGKGLEWIGR |
| Danio_rerio_IGHV1-3*01 | MHWIRQKPGKGLEWIGG |
| Danio_rerio_IGHV1-4*01 | LHWIRQKPGKGLEWIGR |
| Danio_rerio_IGHV10-1*01 | MAWIRQKSGKELEWLAL |
| Danio_rerio_IGHV11-1*01 | ISWIRQAEGKALEWISH |
| Danio_rerio_IGHV11-2*01 | ISWIRQAEGKGLEWISH |
| Danio_rerio_IGHV13-2*01 | TAWIRHPPGKAMEWIGI |
| Danio_rerio_IGHV14-1*01 | MAWIRQAAGKGPEWVAT |
| Danio_rerio_IGHV2-1*01 | MHWIRQKPGKALEWIGR |
| Danio_rerio_IGHV2-3*01 | MHWIRQKPGEALEWIGK |
| Danio_rerio_IGHV3-2*01 | MSWIRQTAGGGLEWLAY |
| Danio_rerio_IGHV4-1*01 | MHWIRQQPGKALMWMGR |
| Danio_rerio_IGHV4-2*01 | MHWIRQQAGKPLVWMGL |
| Danio_rerio_IGHV4-3*01 | MHWIRQQAGKPLMWIGR |
| Danio_rerio_IGHV4-5*01 | MHWIRQPAGKALDWIGV |
| Danio_rerio_IGHV4-6*01 | MHWIRQQAGKPLVWIGG |
| Danio_rerio_IGHV4-7*01 | MQWIRQPAGKALDWIGV |
| Danio_rerio_IGHV4-8*01 | MHWIRQQAGKPLVWIGY |
| Danio_rerio_IGHV4-9*01 | MHWIRQQAGKPLVWIGR |
| Danio_rerio_IGHV5-1*01 | TAWIRQPAGKALEWIGV |
| Danio_rerio_IGHV5-3*01 | THWIRQAAGKALEWIGQ |
| Danio_rerio_IGHV6-1*01 | LHWIRQSPGKALEWIAY |
| Danio_rerio_IGHV7-1*01 | INWIRQSAGRPLEWLGY |
| Danio_rerio_IGHV8-1*01 | TNWIRHSAGKALEWIGW |
| Danio_rerio_IGHV8-2*01 | TNWIRHSAGKPMEWIGY |
| Danio_rerio_IGHV8-4*01 | TNWIRHSAGKPMEWIGW |
| Danio_rerio_IGHV9-1*01 | MHWIRQAPGKGLEWILH |
| Danio_rerio_IGHV9-2*01 | MYWIRQAPGKGLEWIIY |
| Danio_rerio_IGHV9-3*01 | MFWIHQTPGKGLELILN |
| Danio_rerio_IGHV9-4*01 | MHWIRQAPGKGLEWIIY |
| HORSE | |
| Equus_caballus_IGHV1-41*01 | MSWVRQAPGQGLDWMGG |
| Equus_caballus_IGHV1-5*01 | MSWVRQAPGQGLEWTGY |
| Equus_caballus_IGHV1-70*01 | MHWVRQANGKGIEWMGS |
| Equus_caballus_IGHV2-63*01 | VGWMRQPPGKALEWLTY |
| Equus_caballus_IGHV3-78*01 | MSWVRQAPGKGLQWVAG |
| Equus_caballus_IGHV4-11*01 | VHWTHQAPGKGLEWLGD |
| Equus_caballus_IGHV4-17*01 | WSWIRQPPGKGLEWMGY |
| Equus_caballus_IGHV4-22*01 | VGWVCQAPGKGLEYVGV |
| Equus_caballus_IGHV4-29*01 | VGWVRQAPGKGLEYVGA |
| Equus_caballus_IGHV4-37*01 | WSWIRQTPGKGLEYIGS |
| Equus_caballus_IGHV4-55*01 | WSWIRQPPGKRLEWMGS |
| Equus_caballus_IGHV4-59*01 | WNWIRQPPNKGLEWMGS |
| Equus_caballus_IGHV4-65*01 | WSWLRQPPGKGLEYMGY |
| Equus_caballus_IGHV4-75*01 | WSWTRQPLRKGLEWMGL |
| Equus_caballus_IGHV4S1*01 | VGWVRQAPGKGLEWVGV |
| Equus_caballus_IGHV9-66*01 | LWWIRLLPGKGLECVGV |
| JUNGLE FOWL | |
| Gallus_gallus_IGHV1-1*01 | MGWVRQAPGKGLEWVAG |
| Gallus_gallus_IGHV1S1*01 | MGWVRQAPGKGLEFVAG |
| GORILLA | |
| Gorilla_gorilla_IGHV1-2*01 | MHWVRQAPGQGLEWMGW |
| Gorilla_gorilla_IGHV1-3*01 | MHWVRQAPGQRLEWMGW |
| Gorilla_gorilla_IGHV1-45*01 | LHWVRQAPGQALEWMGW |
| Gorilla_gorilla_IGHV1-46*01 | MHWVRQAPGQGLEWMGI |
| Gorilla_gorilla_IGHV1-69*01 | ISWVRQAPGQGLEWMGG |
| Gorilla_gorilla_IGHV1-8*01 | INWVRQAPGQGLEWMGW |
| Gorilla_gorilla_IGHV2-26*01 | VSWIRQPPGKALEWLAH |
| Gorilla_gorilla_IGHV2-70*01 | VGWIRQPLGKALEWLAR |
| Gorilla_gorilla_IGHV3-11*01 | MSWVRQAPGKGLEWVSY |
| Gorilla_gorilla_IGHV3-15*01 | MNWVRQAPGKGLEWVGR |
| Gorilla_gorilla_IGHV3-20*01 | MSWVRQAPGKGLEWVSG |
| Gorilla_gorilla_IGHV3-22*01 | MEWVRQAPGKGLEWVGF |
| Gorilla_gorilla_IGHV3-23*01 | MSWVRQAPGKGLEWVST |
| Gorilla_gorilla_IGHV3-33*01 | MHWVRQAPGKGLEWVAV |
| Gorilla_gorilla_IGHV3-38*01 | ISWVRQAPGKGLEWVSV |
| Gorilla_gorilla_IGHV3-41D*01 | MDWVRQAPGKGLEWVAH |
| Gorilla_gorilla_IGHV3-49*01 | MSWVRQAPGKGLEWVGF |
| Gorilla_gorilla_IGHV3-7*01 | MHWVRQTPGKGLEWVAN |
| Gorilla_gorilla_IGHV3-71*01 | KSWVCQAPGKGLEWVRF |
| Gorilla_gorilla_IGHV3-72*01 | MDWVRQAPGKGPEWVGR |
| Gorilla_gorilla_IGHV3-73*01 | MHWVRQASGKGLEWVGR |
| Gorilla_gorilla_IGHV3-9*01 | MHWVRQAPGKGLEWVSV |
| Gorilla_gorilla_IGHV4-31*01 | WSWIRQPPGKGLEWIGL |
| Gorilla_gorilla_IGHV4-34*01 | WNWIRQPPGKGLEWIGH |
| Gorilla_gorilla_IGHV4-39*01 | WSWIRQPPGKGLEWIGS |
| Gorilla_gorilla_IGHV4-4*01 | WIWVRQSPGKGLEWIGY |
| Gorilla_gorilla_IGHV4-55*01 | WIWVRQPPGKGLEWIGE |
| Gorilla_gorilla_IGHV4-59*01 | WSWIRQPPGKGLEWIGY |
| Gorilla_gorilla_IGHV4-61*01 | WSWIRQPAGKGLEWIGR |
| Gorilla_gorilla_IGHV4-80*01 | WSWVRQSPGKGLEWIGY |
| Gorilla_gorilla_IGHV6-1*01 | WNWIRQSPSRGLEWLGR |
| Gorilla_gorilla_IGHV7-4-1*01 | MNWVRQAPGQGLEWMGW |
| HUMAN | |
| Homo_sapiens_IGHV1-18*01 | ISWVRQAPGQGLEWMGW |
| Homo_sapiens_IGHV1-2*01 | MHWVRQAPGQGLEWMGR |
| Homo_sapiens_IGHV1-24*01 | MHWVRQAPGKGLEWMGG |
| Homo_sapiens_IGHV1-3*01 | MHWVRQAPGQRLEWMGW |
| Homo_sapiens_IGHV1-45*01 | LHWVRQAPGQALEWMGW |
| Homo_sapiens_IGHV1-46*01 | MHWVRQAPGQGLEWMGI |
| Homo_sapiens_IGHV1-58*01 | VQWVRQARGQRLEWIGW |
| Homo_sapiens_IGHV1-69*01 | ISWVRQAPGQGLEWMGG |
| Homo_sapiens_IGHV1-69-2*01 | MHWVQQAPGKGLEWMGL |
| Homo_sapiens_IGHV1-69D*01 | ISWVRQAPGQGLEWMGG |
| Homo_sapiens_IGHV1-8*01 | INWVRQATGQGLEWMGW |
| Homo_sapiens_IGHV2-26*01 | VSWIRQPPGKALEWLAH |
| Homo_sapiens_IGHV2-5*01 | VGWIRQPPGKALEWLAL |
| Homo_sapiens_IGHV2-70*01 | VSWIRQPPGKALEWLAL |
| Homo_sapiens_IGHV3-11*01 | MSWIRQAPGKGLEWVSY |
| Homo_sapiens_IGHV3-13*01 | MHWVRQATGKGLEWVSA |
| Homo_sapiens_IGHV3-15*01 | MSWVRQAPGKGLEWVGR |
| Homo_sapiens_IGHV3-20*01 | MSWVRQAPGKGLEWVSG |
| Homo_sapiens_IGHV3-21*01 | MNWVRQAPGKGLEWVSS |
| Homo_sapiens_IGHV3-23*01 | MSWVRQAPGKGLEWVSA |
| Homo_sapiens_IGHV3-33*01 | MHWVRQAPGKGLEWVAV |
| Homo_sapiens_IGHV3-43*01 | MHWVRQAPGKGLEWVSL |
| Homo_sapiens_IGHV3-48*01 | MNWVRQAPGKGLEWVSY |
| Homo_sapiens_IGHV3-49*01 | MSWFRQAPGKGLEWVGF |
| Homo_sapiens_IGHV3-53*01 | MSWVRQAPGKGLEWVSV |
| Homo_sapiens_IGHV3-64*01 | MHWVRQAPGKGLEYVSA |
| Homo_sapiens_IGHV3-7*01 | MSWVRQAPGKGLEWVAN |
| Homo_sapiens_IGHV3-72*01 | MDWVRQAPGKGLEWVGR |
| Homo_sapiens_IGHV3-73*01 | MHWVRQASGKGLEWVGR |
| Homo_sapiens_IGHV3-74*01 | MHWVRQAPGKGLVWVSR |
| Homo_sapiens_IGHV3-9*01 | MHWVRQAPGKGLEWVSG |
| Homo_sapiens_IGHV4-28*01 | WGWIRQPPGKGLEWIGY |
| Homo_sapiens_IGHV4-31*01 | WSWIRQHPGKGLEWIGY |
| Homo_sapiens_IGHV4-34*01 | WSWIRQPPGKGLEWIGE |
| Homo_sapiens_IGHV4-4*01 | WSWVRQPPGKGLEWIGE |
| Homo_sapiens_IGHV5-10-1*01 | ISWVRQMPGKGLEWMGR |
| Homo_sapiens_IGHV5-51*01 | IGWVRQMPGKGLEWMGI |
| Homo_sapiens_IGHV6-1*01 | WNWIRQSPSRGLEWLGR |
| Homo_sapiens_IGHV7-4-1*01 | MNWVRQAPGQGLEWMGW |
| MOUSE | |
| Mus_musculus_IGHV1-11*01 | MNWVKKRPGQGLEWIGR |
| Mus_musculus_IGHV1-12*01 | MHWVKQTPRQGLEWIGA |
| Mus_musculus_IGHV1-15*01 | MHWVKQTPVHGLEWIGA |
| Mus_musculus_IGHV1-18*01 | MDWVKQSHGKSLEWIGD |
| Mus_musculus_IGHV1-19*01 | MNWVKQSHGKSLEWIGV |
| Mus_musculus_IGHV1-20*01 | MNWVMQSHGKSLEWIGR |
| Mus_musculus_IGHV1-22*01 | MHWVKQSHGKSLEWIGY |
| Mus_musculus_IGHV1-26*01 | MNWVKQSHGKSLEWIGD |
| Mus_musculus_IGHV1-31*01 | MHWVKQSHGNILDWIGY |
| Mus_musculus_IGHV1-36*01 | MHWVKQSHGKSLEWIGL |
| Mus_musculus_IGHV1-37*01 | MNWVKQSHGKSLEWIGR |
| Mus_musculus_IGHV1-39*01 | MNWVKQSNGKSLEWIGV |
| Mus_musculus_IGHV1-4*01 | MHWVKQRPGQGLEWIGY |
| Mus_musculus_IGHV1-42*01 | MNWVKQSPEKSLEWIGE |
| Mus_musculus_IGHV1-43*01 | MHWVKQSSEKSLEWIGE |
| Mus_musculus_IGHV1-47*01 | IEWMKQNHGKSLEWIGN |
| Mus_musculus_IGHV1-49*01 | MHWVKQRPGHGLEWIGS |
| Mus_musculus_IGHV1-5*01 | MHWVKQRPGQGLEWIGA |
| Mus_musculus_IGHV1-50*01 | MQWVKQRPGQGLEWIGE |
| Mus_musculus_IGHV1-52*01 | MHWVKQRPIQGLEWIGN |
| Mus_musculus_IGHV1-53*01 | MHWVKQRPGQGLEWIGN |
| Mus_musculus_IGHV1-54*01 | IEWVKQRPGQGLEWIGV |
| Mus_musculus_IGHV1-55*01 | ITWVKQRPGQGLEWIGD |
| Mus_musculus_IGHV1-56*01 | MQWVRQRPGQGLEWIGE |
| Mus_musculus_IGHV1-58*01 | INWVKQRPGQGLEWIGY |
| Mus_musculus_IGHV1-59*01 | MHWVKQRPGQGLEWIGV |
| Mus_musculus_IGHV1-61*01 | MDWVKQRPGQGLEWIGN |
| Mus_musculus_IGHV1-62-1*01 | MQWVKQRPGQGLEWIGE |
| Mus_musculus_IGHV1-62-2*01 | IHWVKQRSGQGLEWIGW |
| Mus_musculus_IGHV1-63*01 | IGWAKQRPGHGLEWIGD |
| Mus_musculus_IGHV1-64*01 | MHWVKQRPGQGLEWIGM |
| Mus_musculus_IGHV1-66*01 | IHWVKQRPGQGLEWIGW |
| Mus_musculus_IGHV1-69*01 | MHWVKQRPGQGLEWIGE |
| Mus_musculus_IGHV1-7*01 | MHWVKQRPGQGLEWIGY |
| Mus_musculus_IGHV1-71*01 | IHWVKQRSGQGLEWIGW |
| Mus_musculus_IGHV1-72*01 | MHWVKQRPGRGLEWIGR |
| Mus_musculus_IGHV1-74*01 | MHWVKQRPGQGLEWIGR |
| Mus_musculus_IGHV1-75*01 | INWVKQRPGQGLEWIGW |
| Mus_musculus_IGHV1-76*01 | INWVKQRPGQGLEWIAR |
| Mus_musculus_IGHV1-77*01 | INWVKQRPGQGLEWIGK |
| Mus_musculus_IGHV1-78*01 | IHWMKQRPEQGLEWIGY |
| Mus_musculus_IGHV1-80*01 | MNWVKQRPGKGLEWIGQ |
| Mus_musculus_IGHV1-81*01 | ISWVKQRTGQGLEWIGE |
| Mus_musculus_IGHV1-82*01 | MNWVKQRPGKGLEWIGR |
| Mus_musculus_IGHV1-84*01 | INWVKQRPGQGLEWIGW |
| Mus_musculus_IGHV1-85*01 | INWVKQRPGQGLEWIGW |
| Mus_musculus_IGHV1-87*01 | MQWVKQRPGQGLEWIGA |
| Mus_musculus_IGHV1-9*01 | IEWVKQRPGHGLEWIGE |
| Mus_musculus_IGHV10-1*01 | MNWVRQAPGKGLEWVAR |
| Mus_musculus_IGHV10-3*01 | MHWVRQAPGKGLEWVAR |
| Mus_musculus_IGHV11-1*01 | MSWVRQTPGKTLEWIGD |
| Mus_musculus_IGHV11-2*01 | MSWVRQTPGKTLEWIGD |
| Mus_musculus_IGHV12-3*01 | WIWIRQSPGKPLEWMGY |
| Mus_musculus_IGHV13-2*01 | MHWLRQPPGKRLEWIAV |
| Mus_musculus_IGHV14-1*01 | MHWVKQRPEQGLEWIGR |
| Mus_musculus_IGHV14-2*01 | MHWVKQRTEQGLEWIGR |
| Mus_musculus_IGHV14-3*01 | MHWVKQRPEQGLEWIGR |
| Mus_musculus_IGHV14-4*01 | MHWVKQRPEQGLEWIGW |
| Mus_musculus_IGHV15-2*01 | MSWVRQKPGHGFEWIGG |
| Mus_musculus_IGHV2-2*01 | VHWVRQSPGKGLEWLGV |
| Mus_musculus_IGHV2-3*01 | VSWVRQPPGKGLEWLGV |
| Mus_musculus_IGHV2-4*01 | VHWVRQPPGKGLEWLGV |
| Mus_musculus_IGHV2-5*01 | VHWVRQSPGKGLEWLGV |
| Mus_musculus_IGHV2-6*01 | VDWVRQSPGKGLEWLGV |
| Mus_musculus_IGHV2-6-5*01 | VSWIRQPPGKGLEWLGV |
| Mus_musculus_IGHV2-7*01 | VHWFRKPPRKGLEWLGG |
| Mus_musculus_IGHV2-9*01 | VDWVRQPPGKGLEWLGV |
| Mus_musculus_IGHV3-1*01 | WHWIRHFPGNKLEWMGY |
| Mus_musculus_IGHV3-3*01 | WIWIRQFPGNKLEYIGY |
| Mus_musculus_IGHV3-4*01 | WNWIRQVSGSKLEWIGY |
| Mus_musculus_IGHV3-5*01 | WSWIRQFPGNKLEWIGY |
| Mus_musculus_IGHV3-6*01 | WNWIRQFPGNKLEWMGY |
| Mus_musculus_IGHV3-8*01 | WNWIRKFPGNKLEYMGY |
| Mus_musculus_IGHV3S1*01 | WNWIRKFPGNKLEYMGY |
| Mus_musculus_IGHV3S7*01 | WNWIRKFPGNKLEYMGF |
| Mus_musculus_IGHV4-1*01 | MSWVRRAPGKGLEWIGE |
| Mus_musculus_IGHV5-9*01 | MSWVRQTPEKRLEWVAT |
| Mus_musculus_IGHV6-3*01 | MNWVRQSPEKGLEWVAQ |
| Mus_musculus_IGHV6-6*01 | MDWVRQSPEKGLEWVAE |
| Mus_musculus_IGHV6-7*01 | MNWFCQSPEKGLEWVAQ |
| Mus_musculus_IGHV7-1*01 | MEWVRQAPGKGLEWIAA |
| Mus_musculus_IGHV7-3*01 | MSWVRQPPGKALEWLGF |
| Mus_musculus_IGHV7-4*01 | MSWVRQPPGKAPEWLAL |
| Mus_musculus_IGHV8-11*01 | VGWIHQPSGNGLEWLAH |
| Mus_musculus_IGHV8-12*01 | VSWIRQPSGKGLEWLAH |
| Mus_musculus_IGHV8-6*01 | VSWIRQPSGKDLEWLAH |
| Mus_musculus_IGHV8-8*01 | VGWIRQPSGKGLEWLAH |
| Mus_musculus_IGHV9-1*01 | MHWVKQAPGKGFKWMGM |
| Mus_musculus_IGHV9-2*01 | MHWVKQAPGKGFKWMGW |
| Mus_musculus_IGHV9-2-1*01 | MHWVKQAPGKGLKWMGW |
| Mus_musculus_IGHV9-3*01 | MSWVKQAPGKGLKWMGW |
| Mus_musculus_IGHV9-3-1*01 | MNWVKQAPGKGLKWMGW |
| Mus_musculus_IGHV9-4*01 | MQWVQKMPGKGFKWIGW |
| RABBIT | |
| Oryctolagus_cuniculus_IGHV1S1*01 | VSWFRQAPGKGLEWIRY |
| Oryctolagus_cuniculus_IGHV1S13*01 | VSWVRQAAGKGLEWIGY |
| Oryctolagus_cuniculus_IGHV1S17*01 | MGWVRQAPGEGLEYIGW |
| Oryctolagus_cuniculus_IGHV1S24*01 | MGWVRQAPGKGLEYIGY |
| Oryctolagus_cuniculus_IGHV1S25*01 | MGWVRQAPGKGLEYIGI |
| Oryctolagus_cuniculus_IGHV1S26*01 | ISWVRQAPGNGLEWIGI |
| Oryctolagus_cuniculus_IGHV1S28*01 | MSWVRQAPGKELEWIGY |
| Oryctolagus_cuniculus_IGHV1S31*01 | VIWVRQAPGNGLEYIGT |
| Oryctolagus_cuniculus_IGHV1S33*01 | MSWVRQAPGKGLEWIGF |
| Oryctolagus_cuniculus_IGHV1S34*01 | ISWVRQAPGNGLEWIGA |
| Oryctolagus_cuniculus_IGHV1S36*01 | VSWVRQAAGKGLEWIGY |
| Oryctolagus_cuniculus_IGHV1S40*01 | MCWVRQAPGKGLEWIAC |
| Oryctolagus_cuniculus_IGHV1S43*01 | ICWVRQAPGKGLELIAC |
| Oryctolagus_cuniculus_IGHV1S44*01 | MGWVRQAPGKGLEYIGI |
| Oryctolagus_cuniculus_IGHV1S45*01 | ICWVRQAPGKGLEWIAC |
| Oryctolagus_cuniculus_IGHV1S47*01 | MCWVRQAPGKGPEWIAC |
| Oryctolagus_cuniculus_IGHV1S49*01 | VSWVRQAPGKGPEWIGA |
| Oryctolagus_cuniculus_IGHV1S50*01 | ISWVRQAPGNGLEYIGY |
| Oryctolagus_cuniculus_IGHV1S51*01 | IGWVRQAPGSGLEWIGI |
| Oryctolagus_cuniculus_IGHV1S52*01 | MGWVRQAPGNELEWIGI |
| Oryctolagus_cuniculus_IGHV1S53*01 | VSWVRQAPGNGLEWIGT |
| Oryctolagus_cuniculus_IGHV1S54*01 | MSWVRQAPGEGLEYIGC |
| Oryctolagus_cuniculus_IGHV1S55*01 | VSWVRQAPGEGLECIGW |
| Oryctolagus_cuniculus_IGHV1S56*01 | MGWVPPAPGKGLEYIGW |
| Oryctolagus_cuniculus_IGHV1S57*01 | IIWVRQAPGNGLEWIGI |
| Oryctolagus_cuniculus_IGHV1S58*01 | ISWVRQAPGNGLEWIGV |
| Oryctolagus_cuniculus_IGHV1S59*01 | TGWVRQAPGKELEVVAY |
| Oryctolagus_cuniculus_IGHV1S60*01 | IHWVRQAPGKGLEWIGM |
| Oryctolagus_cuniculus_IGHV1S61*01 | ISWVRQAPGNGLEWIGA |
| Oryctolagus_cuniculus_IGHV1S62*01 | IFWVRQAPGKELEWIGY |
| Oryctolagus_cuniculus_IGHV1S63*01 | MGWVRQAPGNGLEVIGY |
| Oryctolagus_cuniculus_IGHV1S65*01 | VIWVRQAPGKELEWIGY |
| Oryctolagus_cuniculus_IGHV1S66*01 | VMWVRQAPGNGLEWIGF |
| Oryctolagus_cuniculus_IGHV1S67*01 | VIWVRQAPGNGLEYIGS |
| Oryctolagus_cuniculus_IGHV1S68*01 | VIWVRQAPGSGLEYIGT |
| Oryctolagus_cuniculus_IGHV1S69*01 | MSWVRQAPGKGLEWIGI |
| Oryctolagus_cuniculus_IGHV1S7*01 | MCWVRQAPGKGLEWIGC |
| Oryctolagus_cuniculus_IGHV1S8*01 | MCWVHQAPGKGLEWIAC |
| RAT | |
| Rattus_norvegicus_IGHV1-11*01 | YMHWVNQRPGKGLEWIG |
| Rattus_norvegicus_IGHV1-13*01 | VMNWVKQSPGQGLEWIG |
| Rattus_norvegicus_IGHV1-16*01 | YMHWVKQRPEQGLVWIG |
| Rattus_norvegicus_IGHV1-18*01 | YMYWVKQRPKQGLELIG |
| Rattus_norvegicus_IGHV1-22*01 | YMNWLKQVPGQSIEWIG |
| Rattus_norvegicus_IGHV1-24*01 | YMHWIRQQPGNGLEWIG |
| Rattus_norvegicus_IGHV1-25*01 | YMSWFKQIPGQSIEWIG |
| Rattus_norvegicus_IGHV1-28*01 | DMHWIKQQPGNGLEWIG |
| Rattus_norvegicus_IGHV1-29*01 | YMSWLKQVPGPSIEWIG |
| Rattus_norvegicus_IGHV1-31*01 | YMSWLKQMPGQNIEWIG |
| PIG | |
| Sus_scrofa_IGHV1-10*01 | INWVRQAPGKGLEWLAA |
| Sus_scrofa_IGHV1-11*01 | VGWVRQAPGKGLESLAS |
| Sus_scrofa_IGHV1-14*01 | ISWVRQAPGKGLEWLAA |
| Sus_scrofa_IGHV1-15*01 | MSWVRQAPGKGLEWLAG |
| Sus_scrofa_IGHV1-2*01 | MSWVRQAPGKGLEWLAG |
| Sus_scrofa_IGHV1-4*01 | INWVRQAPGKGLEWLAA |
| Sus_scrofa_IGHV1-5*01 | VSWVRQAPGKGLESLAS |
| Sus_scrofa_IGHV1-6*01 | FSWVRQAPGKGLEWVAA |
| Sus_scrofa_IGHV1-8*01 | VSWVRQAPGKGLEWLAG |
| Sus_scrofa_IGHV1S2*01 | ISWVRQAPGKGLEWLAG |
| Sus_scrofa_IGHV1S6*01 | VGWVRQAPGKGLESLAS |
| ALPACA | |
| Vicugna_pacos_IGHV1-1*01 | IDWVRQAPGQGLGWVGR |
| Vicugna_pacos_IGHV1S2*01 | IDWVRQAPGQGLEWMGR |
| Vicugna_pacos_IGHV3-1*01 | MSWVRQAPGKGLEWVSA |
| Vicugna_pacos_IGHV3-2*01 | MSWVRQAPGKGLESVSS |
| Vicugna_pacos_IGHV3-3*01 | MGWFRQAPGKEREFVAA |
| Vicugna_pacos_IGHV3S1*01 | MYWVRQAPGKGLEWVSA |
| Vicugna_pacos_IGHV3S10*01 | MSWVRQAPGKGLEWVSG |
| Vicugna_pacos_IGHV3S13*01 | MSWVRQAPGKGLESVST |
| Vicugna_pacos_IGHV3S14*01 | MSWVRQAPGKGLESVST |
| Vicugna_pacos_IGHV3S16*01 | MSWVRQAPGKGLEWVSA |
| Vicugna_pacos_IGHV3S17*01 | MSWVRHSPGKGLEWVSA |
| Vicugna_pacos_IGHV3S18*01 | MYWVRQAPGKGLEWVSS |
| Vicugna_pacos_IGHV3S2*01 | MSWVRQAPGKGLEWVSS |
| Vicugna_pacos_IGHV3S20*01 | MSWVHQAPGKGLEWVSS |
| Vicugna_pacos_IGHV3S25*01 | MNWVRQAPGKGLEWVSA |
| Vicugna_pacos_IGHV3S27*01 | MYWVRQAPGKGLEWVSA |
| Vicugna_pacos_IGHV3S28*01 | MSWVRQAPGKGLEWVSA |
| Vicugna_pacos_IGHV3S29*01 | MSWVRQAPGKGLEWVST |
| Vicugna_pacos_IGHV3S30*01 | MSWVRQAPGKGPEWVSA |
| Vicugna_pacos_IGHV3S33*01 | MSWVRQAPGKGPEWVSD |
| Vicugna_pacos_IGHV3S34*01 | MSWVRQAPGKGPEWVSC |
| Vicugna_pacos_IGHV3S35*01 | MSWVRQAPGKGLEWVSY |
| Vicugna_pacos_IGHV3S36*01 | MSWVRQAPGKGLEWVSY |
| Vicugna_pacos_IGHV3S37*01 | MSWVRRAPGKGLEWVSY |
| Vicugna_pacos_IGHV3S38*01 | MSWVRRAPGKGLEWVSY |
| Vicugna_pacos_IGHV3S39*01 | MSWVRQAPGKGLEWVSD |
| Vicugna_pacos_IGHV3S4*01 | MSWVRQAPGKGLEWVSS |
| Vicugna_pacos_IGHV3S40*01 | MSWVRQAPGKGLEWVSA |
| Vicugna_pacos_IGHV3S41*01 | MSWVRQAPGKGLEWVSA |
| Vicugna_pacos_IGHV3S42*01 | MSWVRQAPGKGLEWVSA |
| Vicugna_pacos_IGHV3S43*01 | MSWVRHSPGKGLEWVSA |
| Vicugna_pacos_IGHV3S44*01 | MSWVRQAPGKGLEWVSA |
| Vicugna_pacos_IGHV3S45*01 | MSWVRQAPGKGPEWVSA |
| Vicugna_pacos_IGHV3S46*01 | LSWVCHSPGKGLEWVSA |
| Vicugna_pacos_IGHV3S5*01 | MSWVRQAPGKGLEWVSG |
| Vicugna_pacos_IGHV3S53*01 | MGWYRQAPGKQRELVAA |
| Vicugna_pacos_IGHV3S55*01 | MGWYRQAPGKERELVAA |
| Vicugna_pacos_IGHV3S57*01 | MGWYRQAPGKERELVAA |
| Vicugna_pacos_IGHV3S58*01 | MGWFRQAPGKEREFVAA |
| Vicugna_pacos_IGHV3S6*01 | MNWVRQAPGKGLEWVSG |
| Vicugna_pacos_IGHV3S60*01 | MGWFRQAPGKEREFVSC |
| Vicugna_pacos_IGHV3S61*01 | IGWFRQAPGKEREGVSC |
| Vicugna_pacos_IGHV3S67*01 | MSWVRQAPGKERELVAA |
| Vicugna_pacos_IGHV3S68*01 | MRWFRQAPGKEREWVSC |
| Vicugna_pacos_IGHV3S8*01 | MGWARQVPGKGLEWVSG |
| Vicugna_pacos_IGHV4S1*01 | WSWIRQPPGKGLEWMGV |
| Vicugna_pacos_IGHV4S2*01 | WSWIRQPPGKGLEWMGA |
| CYNOMOLOGOUS MONKEY | |
| Macaca_fascicularis_IGHV1-1*01 | MHWVRQAPGQGLEWIGL |
| Macaca_fascicularis_IGHV1S1*01 | MHWVRQVPGKGLEWMGG |
| Macaca_fascicularis_IGHV1S10*01 | ISWVRQAPGQGLEWMGG |
| Macaca_fascicularis_IGHV1S2*01 | MHWVRQAPGQGLEWMGE |
| Macaca_fascicularis_IGHV1S9*01 | INWVRQAPEQVLDWMGW |
| Macaca_fascicularis_IGHV2-1*01 | VGWIRQPSRKTLEWLAH |
| Macaca_fascicularis_IGHV2S1*01 | VGWIRQPPGKALEWLAS |
| Macaca_fascicularis_IGHV2S2*01 | VGWIRQPPGKALEWLAR |
| Macaca_fascicularis_IGHV3-10*01 | MEWVRQAPGKGLEWVGQ |
| Macaca_fascicularis_IGHV3-11*01 | MSWVCQAPGKGLEWLSD |
| Macaca_fascicularis_IGHV3-12*01 | MDWVRQAPGKGLEWVGF |
| Macaca_fascicularis_IGHV3-14*01 | MHWVRQAPGKGLEWISA |
| Macaca_fascicularis_IGHV3-20*01 | MHWVRQAQGKGLEWVGL |
| Macaca_fascicularis_IGHV3-22*01 | MYWIRQAPGKGLQWVSA |
| Macaca_fascicularis_IGHV3-6*01 | MFWVRQAPGKGLEWVGF |
| Macaca_fascicularis_IGHV3-7*01 | MHWVRQAPGKGLEWVSS |
| Macaca_fascicularis_IGHV3-9*01 | MDWVRQAPGKGLEWVSR |
| Macaca_fascicularis_IGHV3S10*01 | MSWVRQAPGKGLEWVGF |
| Macaca_fascicularis_IGHV3S11*01 | MSWVRQAPGKGLEWVGF |
| Macaca_fascicularis_IGHV3S15*01 | MSWVRQAPGKGLEWVGR |
| Macaca_fascicularis_IGHV3S16*01 | MSWVRQAPGKGLEWVSF |
| Macaca_fascicularis_IGHV3S17*01 | MHWVRQAPGKGLEWVSA |
| Macaca_fascicularis_IGHV3S18*01 | MYWVRQAPGKGLEWVSG |
| Macaca_fascicularis_IGHV3S19*01 | MYWVRQAPGKGLEWVSR |
| Macaca_fascicularis_IGHV3S20*01 | MHWVRQASGKGLEWVGR |
| Macaca_fascicularis_IGHV3S22*01 | MSWVRQAPGKGPEWVGF |
| Macaca_fascicularis_IGHV3S23*01 | MHWVRQAPGKGLESVSV |
| Macaca_fascicularis_IGHV3S25*01 | MYWVRQAPGKGLEWISA |
| Macaca_fascicularis_IGHV3S26*01 | MHWVRQAPGKGLEWVSV |
| Macaca_fascicularis_IGHV3S29*01 | MHWVRQASGKGLEWVSS |
| Macaca_fascicularis_IGHV3S30*01 | MQWVRQAPGKGLEWVGL |
| Macaca_fascicularis_IGHV3S32*01 | MHWVCQAPGKGLEWVAV |
| Macaca_fascicularis_IGHV3S35*01 | MNWIRQAPGKRLEWVTD |
| Macaca_fascicularis_IGHV3S4*01 | MYWVRQAPGKGLEWVGF |
| Macaca_fascicularis_IGHV3S40*01 | MDWVHQAPGKGLEWVGR |
| Macaca_fascicularis_IGHV3S41*01 | MYWVRQAPGKGLEWVSS |
| Macaca_fascicularis_IGHV3S43*01 | MFWVRQAPGKGLEWVSS |
| Macaca_fascicularis_IGHV3S44*01 | LIWIRQAPGKGLEWVSY |
| Macaca_fascicularis_IGHV3S45*01 | MNWVRQAPGKGLVWVAR |
| Macaca_fascicularis_IGHV3S46*01 | MYWVRQAPGKGLEWVSR |
| Macaca_fascicularis_IGHV3S5*01 | MSWVRQAPGKGLEWVSA |
| Macaca_fascicularis_IGHV4-2*01 | WSWIRQPPGKGLEWIGR |
| Macaca_fascicularis_IGHV4S10*01 | WSWIRQSPGKGLEWIGY |
| Macaca_fascicularis_IGHV4S11*01 | WSWIRQPPGKGLEWIGY |
| Macaca_fascicularis_IGHV4S12*01 | WSWIRQPPGKGLEWIGG |
| Macaca_fascicularis_IGHV4S13*01 | WSWIRQPPGKGLEWIGR |
| Macaca_fascicularis_IGHV4S14*01 | WGWIRQPPGKGLEWIGS |
| Macaca_fascicularis_IGHV4S15*01 | WGWIRQPPGKGLEYIGY |
| Macaca_fascicularis_IGHV4S16*01 | WSWIRQPPGKGLEWIGN |
| Macaca_fascicularis_IGHV4S17*01 | WSWIRQPPGKGLEWIGN |
| Macaca_fascicularis_IGHV4S18*01 | WSWIRQPPGKGLEWIGY |
| Macaca_fascicularis_IGHV4S19*01 | WSWIRQPPGKGLEWIGN |
| Macaca_fascicularis_IGHV4S5*01 | WNWIRQPPGKGLEWIGY |
| Macaca_fascicularis_IGHV4S6*01 | WSWIRQPPGKGLEWIGR |
| Macaca_fascicularis_IGHV4S7*01 | WSWIRQPPGKGLEWIGY |
| Macaca_fascicularis_IGHV4S9*01 | WSWIRQSPGKGLEWIGG |
| Macaca_fascicularis_IGHV5-2*01 | ISWVRQMPGKGLEWMGA |
| Macaca_fascicularis_IGHV5S1*01 | ISWVRQMPGKGLEWMGS |
| Macaca_fascicularis_IGHV6-1*01 | WNWIRQSPSRGLEWLGR |
| Macaca_fascicularis_IGHV7-1*01 | MNWVRQAPGQGLEWMGW |
| Macaca_fascicularis_IGHV7S1*01 | MNWLRQAPGQRLEWMGG |

TABLE 5
FR2 Sequences in IGKV (light chain) From Representative Species

| | |
|---|---|
| BOVINE | |
| Bos_taurus_IGKV1-4*01 | LNWYQQKPGEAPKLLIY |
| Bos_taurus_IGKV2-15*01 | LRWVQHKPGQSPQGVIY |
| Bos_taurus_IGKV2-18*01 | LRWLQHKPGQSPQSLIY |
| Bos_taurus_IGKV2-6*01 | LNWIQYKPGQSPQGLIY |
| Bos_taurus_IGKV2-9*01 | LQWFQHKPGQSPRLLIY |
| Bos_taurus_IGKV8-3*01 | LSWYKQKPGQAPMILIY |
| CAMEL | |
| Camelus_dromedarius_IGKV1-26*01 | LAWYQQKPGQVPKLLIY |
| Camelus_dromedarius_IGKV1-29*01 | LSWYQQKPGQAPKLLIY |
| Camelus_dromedarius_IGKV2-28*01 | LYWLLQKPGQPPQRLIY |
| Camelus_dromedarius_IGKV2-30*01 | LSWFLQKPGQSPQLLIY |
| Camelus_dromedarius_GKV4-4*01 | LAWYQQRPGQSPRLLIY |
| Camelus_dromedarius_IGKV5-2*01 | IMWYQQKPGQAPRLIIK |
| Camelus_dromedarius_IGKV8-10*01 | VSWYQQKPGQAPRLLIY |
| Camelus_dromedarius_IGKV9-27*01 | MAWYQQKPKEAPRLLIF |
| DOG | |
| Canis_lupus_familiaris_IGKV2-11*01 | LYWFRQKPGQSPQRLIY |
| Canis_lupus_familiaris_IGKV2-12*01 | LDWYMQKPGQSPQGLIY |
| Canis_lupus_familiaris_IGKV2-16*01 | LNWYLQKPGQTPRPLIY |
| Canis_lupus_familiaris_IGKV2-4*01 | LDWYLQKPGQSPQLLIY |
| Canis_lupus_familiaris_IGKV2-6*01 | LNWFRQKPGQSPQRLIY |
| Canis_lupus_familiaris_IGKV2-7*01 | LNWFRQKPGQSPQGLIY |
| Canis_lupus_familiaris_IGKV2-9*01 | LNWFRQKPGQSPQRLIY |
| Canis_lupus_familiaris_IGKV2S13*01 | LYWFRQKPGQSPQGLIY |
| Canis_lupus_familiaris_IGKV3-18*01 | LAWYQQKPGQAPKLLIY |
| Canis_lupus_familiaris_IGKV4-15*01 | LAWYQQKPGERPKLLIY |
| GOAT | |
| Capra_hircus_IGKV1-4*01 | LNWYQQKPGQAPKLLIY |
| Capra_hircus_IGKV2-14*01 | LYWFQHKPGQSPRRLIY |
| Capra_hircus_IGKV2-8*01 | LNWIKHKPGQSPEGLIY |
| Capra_hircus_IGKV2-9*01 | LHWYVQKPSQSPQLLIY |
| HORSE | |
| Equus_caballus_IGKV1-36*01 | LAWYQQKPGKALKFLIS |
| Equus_caballus_IGKV2-28*01 | LDWYLQKPGQSPRLLIY |
| Equus_caballus_IGKV2-33*01 | LHWFLQKPGQSPRRLTY |
| Equus_caballus_IGKV2-39*01 | LYWYLQKPGQSPKLLIY |
| Equus_caballus_IGKV2-45*01 | LYWFLQKPGQSPKCLIY |
| Equus_caballus_IGKV2-46*01 | LYWFLQKPGQSPKLLIY |
| Equus_caballus_IGKV2-48*01 | LHWYLQKPGQSLQRLIS |
| Equus_caballus_IGKV4-1*01 | LAWYQHKPGQAPKRLIY |
| Equus_caballus_IGKV4-12*01 | LAWYQQKPGQAPKPLIY |
| Equus_caballus_IGKV4-18*01 | LDWYQQKPGEAPMLLIY |
| Equus_caballus_IGKV4-2*01 | LDWHQHKPGQAPKLLIY |
| Equus_caballus_IGKV4-5*01 | LDWYQQKPVKAPKLLIY |
| Equus_caballus_IGKV4-5-1*01 | LDWHQHKPGQAPKQLIY |
| Equus_caballus_IGKV4-8*01 | LSWYQQKPGQAPKLLIY |
| Equus_caballus_IGKV4-9*01 | LAWYQQKLGQAPKLLIY |
| Equus_caballus_IGKV4-9-1*01 | LDWYQQKPGQAPKQLIY |
| Equus_caballus_IGKV5-43*01 | VNWYQWKPGEAPKLIIK |
| CAT | |
| Felis_catus_IGKV1-10*01 | LAWYQQKPGKVPKLLIY |
| Felis_catus_IGKV2-14*01 | LSWYLQKTGQSPQRPIY |
| Felis_catus_IGKV2-4*01 | LNWYLQKPGQSPRRLIY |
| Felis_catus_IGKV2-9*01 | LHWYLQKPGQSPRRLIY |
| Felis_catus_IGKV4-1*01 | LAWYQQKPGQHPKLLIY |
| Felis_catus_IGKV6-6*01 | LHWYQQKPNQAPKLLVK |
| Felis_catus_IGKV8-3*01 | LSWYQQKPGQPPKLIIY |
| HUMAN | |
| Homo_sapiens_IGKV1-12*01 | LAWYQQKPGKAPKLLIY |
| Homo_sapiens_IGKV1-16*01 | LAWFQQKPGKAPKSLIY |
| Homo_sapiens_IGKV1-17*01 | LGWYQQKPGKAPKRLIY |
| Homo_sapiens_IGKV1-27*01 | LAWYQQKPGKVPKLLIY |
| Homo_sapiens_IGKV1-39*01 | LNWYQQKPGKAPKLLIY |
| Homo_sapiens_IGKV1-5*01 | LAWYQQKPGKAPKLLIY |
| Homo_sapiens_IGKV1-6*01 | LGWYQQKPGKAPKLLIY |
| Homo_sapiens_IGKV2-24*01 | LSWLQQRPGQPPRLLIY |
| Homo_sapiens_IGKV2-28*01 | LDWYLQKPGQSPQLLIY |
| Homo_sapiens_IGKV2-30*01 | LNWFQQRPGQSPRRLIY |
| Homo_sapiens_IGKV3-11*01 | LAWYQQKPGQAPRLLIY |
| Homo_sapiens_IGKV4-1*01 | LAWYQQKPGQPPKLLIY |
| Homo_sapiens_IGKV5-2*01 | MNWYQQKPGEAAIFIIQ |
| Homo_sapiens_IGKV6-21*01 | LHWYQQKPDQSPKLLIK |
| MOUSE | |
| Mus_musculus_IGKV1-110*01 | LHWYLQKPGQSPKLLIY |
| Mus_musculus_IGKV1-117*01 | LEWYLQKPGQSPKLLIY |
| Mus_musculus_IGKV1-122*01 | LNWYLQKPGQSPQLLIY |
| Mus_musculus_IGKV1-132*01 | LNWLQQRPGQAPKHLMY |
| Mus_musculus_IGKV1-133*01 | LNWLLQRPGQSPKRLIY |
| Mus_musculus_IGKV1-88*01 | LSWYLHKPGQSPQLLIY |
| Mus_musculus_IGKV1-99*01 | LDWYLQKPGQSPQLLIY |
| Mus_musculus_IGKV10-94*01 | LNWYQQKPDGTVKLLIY |
| Mus_musculus_IGKV11-125*01 | LNWFQQKPGKAPKLLIY |
| Mus_musculus_IGKV12-104-2*01 | LLWYQQKTGNPPKILIY |
| Mus_musculus_IGKV12-38*01 | LAWYQQKQGKSPQLLIY |
| Mus_musculus_IGKV12-41*01 | LAWYQQKQGKSPQLLVY |
| Mus_musculus_IGKV12-89*01 | LNWYQRKQGKSPQLLIY |
| Mus_musculus_IGKV12-98*01 | LAWYQQKPGKSPQLLIY |
| Mus_musculus_IGKV13-84*01 | LAWYQQKPGNAPRLLIS |
| Mus_musculus_IGKV14-100*01 | IGWLQQKPGKSFKGLIY |
| Mus_musculus_IGKV14-111*01 | LSWFQQKPGKSPKTLIY |
| Mus_musculus_IGKV14-126*01 | LSWYQQKPWKSPKTLIY |
| Mus_musculus_IGKV14-130*01 | LNWYQQKPGKPPSFLIY |
| Mus_musculus_IGKV14-87-2*01 | FAWVQQKPRKAPRMLIH |
| Mus_musculus_IGKV16-104*01 | LAWYQEKPGKTNKLLIY |
| Mus_musculus_IGKV17-121*01 | MNWYQQKPGEPPKLLIS |
| Mus_musculus_IGKV18-36*01 | LAWYQQKAEQVPRLLIH |
| Mus_musculus_IGKV19-93*01 | IAWYQHKPGKGPRLLIH |
| Mus_musculus_IGKV2-109*01 | LYWYLQKPGQSPQLLIY |
| Mus_musculus_IGKV2-112*01 | LNWFLQRPGQSPQLLIY |
| Mus_musculus_IGKV2-116*01 | LYWYLQRPGQSPQLLIY |
| Mus_musculus_IGKV2-137*01 | LYWFLQRPGQSPQLLIY |
| Mus_musculus_IGKV2-a*01 | LYWFLQKPGQSPQLLIY |
| Mus_musculus_IGKV20-101-2*01 | IAWYQQKVGKAPILLIR |
| Mus_musculus_IGKV3-1*01 | MQWYQQKPGQPPKLLIY |
| Mus_musculus_IGKV3-10*01 | MHWYQQKPGQPPKLLIY |
| Mus_musculus_IGKV3-2*01 | MNWFQQKPGQPPKLLIY |
| Mus_musculus_IGKV3-3*01 | MHWFQQKPGQPPKLLIY |
| Mus_musculus_IGKV3-4*01 | MNWYQQKPGQPPKLLIY |
| Mus_musculus_IGKV3-5*01 | MHWYQQKPGQPPKLLIY |
| Mus_musculus_IGKV3-7*01 | MHWYQQKPGQPPKLLIK |
| Mus_musculus_IGKV4-50*01 | MYWYQQKSDASPKLWIY |
| Mus_musculus_IGKV4-51*01 | LHWYQQKSGTSPKLWIY |
| Mus_musculus_IGKV4-53*01 | LHWYQQKSETSPKPWIY |
| Mus_musculus_IGKV4-55*01 | MYWYQQKPGSSPRLLIY |
| Mus_musculus_IGKV4-57*01 | MHWFQQKPGTSPKLWIY |
| Mus_musculus_IGKV4-57-1*01 | LHWYQQKSGASPKLWIY |
| Mus_musculus_IGKV4-58*01 | LHWYQQKSGASPKPLIH |
| Mus_musculus_IGKV4-59*01 | MHWYQQKSGTSPKRWIY |
| Mus_musculus_IGKV4-61*01 | MYWYQQKPGSSPKPWIY |
| Mus_musculus_IGKV4-63*01 | MHWYQQKSSTSPKLWIY |
| Mus_musculus_IGKV4-68*01 | MYWYQQKPRSSPKPWIY |
| Mus_musculus_IGKV4-69*01 | MHWYQQKPGSSPKPWIY |
| Mus_musculus_IGKV4-70*01 | MHWYQQKPGTSPKRWIY |
| Mus_musculus_IGKV4-71*01 | MHWYQQKPGSSPRLWIY |
| Mus_musculus_IGKV4-74*01 | LHWYQQKPGSSPKLWIY |
| Mus_musculus_IGKV4-78*01 | LYWYQQKPGSSPKLWIY |
| Mus_musculus_IGKV4-79*01 | LYWYQQKPGSSPKLWIY |
| Mus_musculus_IGKV4-80*01 | MHWYQQKSGTSPKLLIY |
| Mus_musculus_IGKV4-81*01 | LHWYQQKSGTSTKFWIY |
| Mus_musculus_IGKV4-86*01 | MHWYQQKSGTSPKPWIY |
| Mus_musculus_IGKV4-90*01 | MNWYQQKPGSSPKIWIY |
| Mus_musculus_IGKV4-91*01 | LHWYQQKPGFSPKLLIY |
| Mus_musculus_IGKV4-92*01 | LHWYQQKPGSSPKLLIY |
| Mus_musculus_IGKV5-37*01 | LHWYQQKSHRSPRLLIK |
| Mus_musculus_IGKV5-39*01 | LHWYQQKSHESPRLLIK |
| Mus_musculus_IGKV5-48*01 | IHWYQQRTNGSPRLLIK |
| Mus_musculus_IGKV6-13*01 | VAWYQQKPGQSPKLLIY |
| Mus_musculus_IGKV6-20*01 | VSWYQQKPEQSPKLLIY |
| Mus_musculus_IGKV7-33*01 | LAWYQKKPEQSPKLLIY |
| Mus_musculus_IGKV8-16*01 | LVWHQWKPGQTPTPLIT |
| Mus_musculus_IGKV8-19*01 | LTWYQQKPGQPPKLLIY |
| Mus_musculus_IGKV8-21*01 | LAWYQQKPGQSPKLLIY |
| Mus_musculus_IGKV8-24*01 | LAWYQQKPGQSPKLLVY |
| Mus_musculus_IGKV8-28*01 | LAWYQQKPGQPPKLLIY |
| Mus_musculus_IGKV8-34*01 | LAWHQQKPGRSPKMLII |
| Mus_musculus_IGKV9-120*01 | LNWLQQEPDGTIKRLIY |
| Mus_musculus_IGKV9-123*01 | LDWYQQKPGGTIKLLIY |
| Mus_musculus_IGKV9-124*01 | LSWLQQKPDGTIKRLIY |
| RABBIT | |
| Oryctolagus_cuniculus_IGKV1S1*01 | LSWFQQKPGQPPKLLIY |
| Oryctolagus_cuniculus_IGKV1S2*01 | LSWYQQKPGQPPKLLIY |
| Oryctolagus_cuniculus_IGKV1S3*01 | LNWYQQKLGQPPKLLIY |
| Oryctolagus_cuniculus_IGKV1S5*01 | LSWFQQKPGQPPKLLIY |
| Oryctolagus_cuniculus_IGKV1S6*01 | LSWYQQKPGQPPKLLIY |
| SHEEP | |
| Ovis_aries_IGKV1-4*01 | LNWYQQKPGQAPKLLIY |
| Ovis_aries_IGKV2-14*01 | LYWFQHKPGQSPQLLIY |
| Ovis_aries_IGKV2-8*01 | LNWIQHKPGQSPQGLIY |
| Ovis_aries_IGKV6-5*01 | LHWYQQKSNQAPKFLIK |
| Ovis_aries_IGKV8-3*01 | LSWHKQKPGQAPMILIY |
| RAT | |
| Rattus_norvegicus_IGKV10S11*01 | LNWYQKNPDGTVQLLIY |
| Rattus_norvegicus_IGKV10S12*01 | LNWYQQKPDGTVKRLIY |
| Rattus_norvegicus_IGKV10S5*01 | LNWYQQKPDGTIKPLIY |
| Rattus_norvegicus_IGKV10S6*01 | MAWYQQKPGEVPQLLIY |
| Rattus_norvegicus_IGKV10S9*01 | LNWYQQKPDGTIKPLIY |
| Rattus_norvegicus_IGKV12S1*01 | LLWYQQKPGKSPQLLIY |
| Rattus_norvegicus_IGKV12S11*01 | LAWYQQKPGKSPQLLIY |
| Rattus_norvegicus_IGKV12S14*01 | LAWYQQKPGKSPQLLIY |
| Rattus_norvegicus_IGKV12S16*01 | LSWYQQKPGKSPQLLIY |
| Rattus_norvegicus_IGKV12S17*01 | LAWYQQKPGKSPQLLIY |
| Rattus_norvegicus_IGKV12S20*01 | LAWYQQKPGKSPQLLIY |
| Rattus_norvegicus_IGKV12S22*01 | LSWYQQKLGKSPQLLIH |
| Rattus_norvegicus_IGKV12S24*01 | LSWYQQKPGKSPQLLIH |
| Rattus_norvegicus_IGKV12S25*01 | LAWYQQKPGKSPQLLIY |
| Rattus_norvegicus_IGKV12S26*01 | LAWYQQKPGKSPQLLIY |
| Rattus_norvegicus_IGKV12S29*01 | LAWYQQKPGKSPQLLIY |
| Rattus_norvegicus_IGKV12S30*01 | LAWYQQKPGKSPQLLIY |
| Rattus_norvegicus_IGKV12S31*01 | LAWYQQKPGKSPQLLIY |
| Rattus_norvegicus_IGKV12S32*01 | LAWYQQKPGKSPQLLIY |
| Rattus_norvegicus_IGKV12S34*01 | LAWYQQKPGKSPQLLIY |
| Rattus_norvegicus_IGKV12S36*01 | LAWYQQKPGKSPQLLIY |
| Rattus_norvegicus_IGKV12S38*01 | LAWYQQKSGKSPQLLIY |
| Rattus_norvegicus_IGKV12S39*01 | LAWYQQKPGKSPQLLIY |
| Rattus_norvegicus_IGKV12S7*01 | LLWYQQKPGKSPQLLIY |
| Rattus_norvegicus_IGKV12S8*01 | LAWYQQKSGKSPQLLIY |
| Rattus_norvegicus_IGKV12S9*01 | LLWYQQKPGKSPQLLIY |
| Rattus_norvegicus_IGKV14S1*01 | LIWFQQKPGKSPRPMIY |
| Rattus_norvegicus_IGKV14S13*01 | LRWFQQKPGKSPRLMIY |
| Rattus_norvegicus_IGKV14S14*01 | LTWFQQKPGKSPRRMIY |
| Rattus_norvegicus_IGKV14S15*01 | LSWFQQKPGKSPRRMIY |
| Rattus_norvegicus_IGKV14S16*01 | LSWYQQKPGKSPKLMIS |
| Rattus_norvegicus_IGKV14S18*01 | LRWFQQKPGKSPRLMIY |
| Rattus_norvegicus_IGKV14S19*01 | LSWYQQKPEKSPKLMIY |
| Rattus_norvegicus_IGKV14S2*01 | LRWFQQKPGKSPRRMIY |
| Rattus_norvegicus_IGKV14S22*01 | LAWYQQKRGKAPKLLIY |
| Rattus_norvegicus_IGKV14S8*01 | LSWYQQKPGKSPKLMIY |
| Rattus_norvegicus_IGKV14S9*01 | VNWFQQKPGKSPRHMIY |
| Rattus_norvegicus_IGKV15S4*01 | LAWYQQKSGNAPELLIY |
| Rattus_norvegicus_IGKV16S1*01 | LAWYQQKPGKANKLLIY |
| Rattus_norvegicus_IGKV17S1*01 | MNWYQQKSGEAPKLLIS |
| Rattus_norvegicus_IGKV18S1*01 | LAWYQQKTGQVPRLLIH |
| Rattus_norvegicus_IGKV19S1*01 | IAWYQQKPGKAPGLLIG |
| Rattus_norvegicus_IGKV19S2*01 | IAWYQQKPGKAPRQLIH |
| Rattus_norvegicus_IGKV1S1*01 | LNWILQRPGQSPKRLIY |
| Rattus_norvegicus_IGKV1S12*01 | LNWLLQNPGQSPKRLIY |
| Rattus_norvegicus_IGKV1S14*01 | LHWLLQSPGRSPKRLIY |
| Rattus_norvegicus_IGKV1S18*01 | LNWLLQRPGQSPKRLIY |
| Rattus_norvegicus_IGKV1S19*01 | LSWYLQKPGQDPQPLIY |
| Rattus_norvegicus_IGKV1S21*01 | LSWYLQKPGQSPQLLIY |
| Rattus_norvegicus_IGKV1S22*01 | LHWYLQKPGQSPQLLIY |
| Rattus_norvegicus_IGKV1S23*01 | LKWYLQKPGQSPQLLIY |
| Rattus_norvegicus_IGKV1S25*01 | LHWYLQKPGQSPQLLIY |
| Rattus_norvegicus_IGKV1S26*01 | LEWYLQKPGQSPQLLIY |
| Rattus_norvegicus_IGKV1S27*01 | LNWYLQKPGQSPQLLIY |
| Rattus_norvegicus_IGKV1S28*01 | LHWFLQKPGQSPQLLIY |
| Rattus_norvegicus_IGKV1S29*01 | LEWYLQKPGQSPQLLIY |
| Rattus_norvegicus_IGKV1S30*01 | LYWYLAKPGQSPQLLIY |
| Rattus_norvegicus_IGKV1S31*01 | LEWYLQKPGQSPQLLIY |
| Rattus_norvegicus_IGKV1S34*01 | LAWYLQKSGQSPQILIY |
| Rattus_norvegicus_IGKV1S42*01 | LSWYLQKPGQSPQLLIY |
| Rattus_norvegicus_IGKV1S5*01 | LNWFLQRPGQSPKCLIY |
| Rattus_norvegicus_IGKV1S7*01 | LNWVFQSPGQSPKRLIY |
| Rattus_norvegicus_IGKV1S8*01 | LSWILQRPGQSPKRLIY |
| Rattus_norvegicus_IGKV20S1*01 | LLWFQQKTGKPPKILIY |
| Rattus_norvegicus_IGKV21S2*01 | LGWYQQKQGQSPKFLMY |
| Rattus_norvegicus_IGKV21S3*01 | LGWYQQKQGQSPKFLMY |
| Rattus_norvegicus_IGKV22S1*01 | LNWYQQKLGEAPKLLIY |
| Rattus_norvegicus_IGKV22S2*01 | LAWYQQKLGEAPKLLIY |
| Rattus_norvegicus_IGKV22S4*01 | LNWYQQKLGEAPKLLIY |
| Rattus_norvegicus_IGKV22S7*01 | LNWYQQKLGEAPKLLIY |
| Rattus_norvegicus_IGKV22S9*01 | LAWYQQKLGEAPKLLIY |
| Rattus_norvegicus_IGKV2S11*01 | LYWYLQRPGKSPQLLIY |
| Rattus_norvegicus_IGKV2S16*01 | LNWFLQRPGQSPQLLTY |
| Rattus_norvegicus_IGKV2S17*01 | VYWYLQKPGKSPQLLIY |
| Rattus_norvegicus_IGKV2S25*01 | LNWLLQRPGQPPQLLIY |
| Rattus_norvegicus_IGKV2S26*01 | LYWFLQRPGQSPQRLIY |
| Rattus_norvegicus_IGKV2S27*01 | LYWYLQRPGQSPQLLIY |
| Rattus_norvegicus_IGKV2S3*01 | LSWYLQRPEKSPQLLIY |
| Rattus_norvegicus_IGKV2S6*01 | LNWYLQRPGQSPQFLIY |
| Rattus_norvegicus_IGKV2S9*01 | VNWYLQKPGKSPQFLIY |
| Rattus_norvegicus_IGKV3S1*01 | MHWYQQKPGQQPKLLIY |
| Rattus_norvegicus_IGKV4S10*01 | MNWHQQKSGTSPKLWIY |
| Rattus_norvegicus_IGKV4S11*01 | MHWYQQKTGTSHKLLIY |
| Rattus_norvegicus_IGKV4S12*01 | MHWFQQKSGTSPKPWIY |
| Rattus_norvegicus_IGKV4S13*01 | MYWYQQKSSASPKLLIY |
| Rattus_norvegicus_IGKV4S14*01 | MYWYQQKSGASPKLWVH |
| Rattus_norvegicus_IGKV4S15*01 | MYWYQQKSGASPKLCIY |
| Rattus_norvegicus_IGKV4S16*01 | MYWYQQMSNASPKLWIY |
| Rattus_norvegicus_IGKV4S18*01 | MNWYQQKSGASPKLWIY |
| Rattus_norvegicus_IGKV4S19*01 | MHWYQQKSGTSPKLLIY |
| Rattus_norvegicus_IGKV4S2*01 | MHWYQQKSGASPKLWIY |
| Rattus_norvegicus_IGKV4S20*01 | MHWYQQKSGASPKLWIY |
| Rattus_norvegicus_IGKV4S21*01 | LHWYQQKPGAFPKLVIY |
| Rattus_norvegicus_IGKV4S3*01 | LHWYQQKAGSYPKLLIY |
| Rattus_norvegicus_IGKV4S4*01 | MYWYQQKSGASPKLLIY |
| Rattus_norvegicus_IGKV4S5*01 | MHWYQQRSGASPKLWIY |
| Rattus_norvegicus_IGKV4S6*01 | MYWYQQKSGASSKLWIY |
| Rattus_norvegicus_IGKV4S7*01 | MYWYQQKSGASPKLWIY |
| Rattus_norvegicus_IGKV4S9*01 | MHWYQQKPGASPKPWIY |
| Rattus_norvegicus_IGKV5S10*01 | IHWYQQKSNESPRLLIK |
| Rattus_norvegicus_IGKV5S12*01 | LHWYQQKPNESPRLLIK |
| Rattus_norvegicus_IGKV5S2*01 | IHWYQQKSNESPRLLIK |
| Rattus_norvegicus_IGKV5S5*01 | LHWYQQKPNESPRVLIK |
| Rattus_norvegicus_IGKV5S6*01 | LHWYQQKTNESPRLLIK |
| Rattus_norvegicus_IGKV6S10*01 | VDWYQQKTGQSPKLLIY |
| Rattus_norvegicus_IGKV6S11*01 | VDWYQQKTGQSPKLLIY |
| Rattus_norvegicus_IGKV6S4*01 | VGWYQQKTGQSPKRLIY |
| Rattus_norvegicus_IGKV6S5*01 | IAWYQQKPGQSPKLLIY |
| Rattus_norvegicus_IGKV6S7*01 | VAWYQQETGQSPKRLIN |
| Rattus_norvegicus_IGKV6S8*01 | VDWYQQTPGQSPKLLIY |
| Rattus_norvegicus_IGKV6S9*01 | VAWFQQKPGQSPKRLIY |
| Rattus_norvegicus_IGKV7S1*01 | LAWYQKKSGQSPKLLIF |
| Rattus_norvegicus_IGKV8S10*01 | LAWYQQKPGQFPKLLIY |
| Rattus_norvegicus_IGKV8S4*01 | LAWYQQKPGQSPKLLIS |
| Rattus_norvegicus_IGKV8S5*01 | LAWYQQKPGQSPKLLIY |
| Rattus_norvegicus_IGKV8S6*01 | LAWYQQKPGQSPKLLIY |
| Rattus_norvegicus_IGKV8S7*01 | LGWYQQKPGQTPKPLIY |
| Rattus_norvegicus_IGKV8S9*01 | LAWYQQKPGQSPKLLIY |
| Rattus_norvegicus_IGKV9S1*01 | LSWYQQRPGQTTKALIY |
| Rattus_norvegicus_IGKV9S2*01 | LSWYQQRPGQTTKALIY |
| PIG | |
| Sus_scrofa_IGKV1-11*01 | LAWYQQQAGKAPKLLIY |
| Sus_scrofa_IGKV1-14*01 | LAWYQQQPGKTPKLLFY |
| Sus_scrofa_IGKV1-7*01 | LAWYQQQPGKPPKLLLC |
| Sus_scrofa_IGKV1-9*01 | LAWYQQQAGKPPKLLIY |
| Sus_scrofa_IGKV1D-11*01 | LAWYQQQAGKAPKLLIY |
| Sus_scrofa_IGKV2-10*01 | LHWYLQKPGQSPQLLIY |
| Sus_scrofa_IGKV2-12*01 | LNWYQQKPGQSPKLLIY |
| Sus_scrofa_IGKV2-13*01 | LSWYQQKPGQSPQLLIY |
| Sus_scrofa_IGKV2-6*01 | LSWYQQKPGQSPRLLIY |
| Sus_scrofa_IGKV2-8*01 | LSWYQQKPGQSPQLLIY |
| Sus_scrofa_IGKV2D-12*01 | LNWYQQKPGQSPKLLIY |
| CYNOMOLOGOUS MONKEY | |
| Macaca_mulatta_IGKV1-101*01 | LAWYQQKPGNSPKLLIY |
| Macaca_mulatta_IGKV1-16*01 | LAWYQQKPGKAPKPLIY |
| Macaca_mulatta_IGKV1-18*01 | LAWYQQKPGKAPKLLIY |
| Macaca_mulatta_IGKV1-21*01 | LAWYQQKPGKAPKLLIY |
| Macaca_mulatta_IGKV1-27*01 | LAWYQQKPGKAPKLLLY |
| Macaca_mulatta_IGKV1-28*01 | LNWFQQKPGKAPKLLIY |
| Macaca_mulatta_IGKV1-32*01 | LNWYQQKPGKAPKLLIY |
| Macaca_mulatta_IGKV1-36*01 | LSWYQQKPGKAPKLLIY |
| Macaca_mulatta_IGKV1-38*01 | LAWYQQKPGKAPKLLIY |
| Macaca_mulatta_IGKV1-41*01 | LNWYQQEPGKAPKLLIY |
| Macaca_mulatta_IGKV1-43*01 | LNWYQQKPGKAPKRLIY |
| Macaca_mulatta_IGKV1-44*01 | LAWYQQKPGKVPKLLIY |
| Macaca_mulatta_IGKV1-46*01 | LAWYQQKPGKAPKLLIY |
| Macaca_mulatta_IGKV1-59*01 | LAWYQQKPGKVPKLLIY |
| Macaca_mulatta_IGKV1-6*01 | LAWYQQKPGKAPKLLIS |
| Macaca_mulatta_IGKV1-66*01 | LSWYQQKPGKAPKPLIY |
| Macaca_mulatta_IGKV1-69*01 | LAWYQQKPGKAPKLLIY |
| Macaca_mulatta_IGKV1-74*01 | LNWYQQKPGKAPKLLIY |
| Macaca_mulatta_IGKV1-80*01 | LAWYQQKPGKAPTLLLY |
| Macaca_mulatta_IGKV1-84*01 | LNWYPQKTWKAPKFLTY |
| Macaca_mulatta_IGKV1-94*01 | LSWYQQKPGKAPTLLIY |
| Macaca_mulatta_IGKV1S11*01 | LAWYQQKPGKAPKLLIY |
| Macaca_mulatta_IGKV1S12*01 | LAWYQQKPGKVPKLLIY |
| Macaca_mulatta_IGKV1S13*01 | LAWYQQKPGKAPKLLIY |
| Macaca_mulatta_IGKV1S14*01 | LAWYQQKPGKAPKPLIY |
| Macaca_mulatta_IGKV1S15*01 | LAWYQQKPGKAPKPLIY |
| Macaca_mulatta_IGKV1S16*01 | LAWYQQKPGKAPKPLIY |
| Macaca_mulatta_IGKV1S17*01 | LAWYQQKPGKAPKLLIY |
| Macaca_mulatta_IGKV1S19*01 | LNWYQQKPGKAPKLLIY |
| Macaca_mulatta_IGKV1S21*01 | LNWYQQKPGKAPKRLIY |
| Macaca_mulatta_IGKV1S25*01 | LAWYQQKPGKVPKLLIY |
| Macaca_mulatta_IGKV1S3*01 | LAWYQQKPGKAPKPLIY |
| Macaca_mulatta_IGKV1S4*01 | LAWYQQKPGKAPKLLLY |
| Macaca_mulatta_IGKV1S5*01 | LAWYQQKPGKAPKRLIY |
| Macaca_mulatta_IGKV1S6*01 | LAWYQQKPGKAPKLLIY |
| Macaca_mulatta_IGKV1S8*01 | LAWYQQKPGKVPKLLIY |
| Macaca_mulatta_IGKV1S9*01 | LNWFQQKPGKAPKLLIY |
| Macaca_mulatta_IGKV2-104*01 | LEWYLQKPGQSPQLLIY |
| Macaca_mulatta_IGKV2-13-1*01 | LDWYLQKPGQSPWLLIY |
| Macaca_mulatta_IGKV2-58*01 | LSWFQQKPGQSPRRLIY |
| Macaca_mulatta_IGKV2-60*01 | LNWYLQKPGQSPQLLIY |
| Macaca_mulatta_IGKV2-61*01 | LDWYLQKPGQSPQLLIY |
| Macaca_mulatta_IGKV2-64*01 | LSWYQQKPGQPPRLLIY |
| Macaca_mulatta_IGKV2-65*01 | LSWYQQKPGQPPRRLIY |
| Macaca_mulatta_IGKV2-68*01 | LDWYLQKPGQSPRFLIY |
| Macaca_mulatta_IGKV2-70*01 | LYWYLAKPGQPPRLLIY |
| Macaca_mulatta_IGKV2-72*01 | LHWYLQKPGQSPQLLIY |
| Macaca_mulatta_IGKV2-73*01 | LYWYLQKPGQPPRLLIY |
| Macaca_mulatta_IGKV2-76*01 | LDWYLQKPGQPPQPLIY |
| Macaca_mulatta_IGKV2-78*01 | LHWYLQKPGQSPQLLIY |
| Macaca_mulatta_IGKV2-82*01 | LDWYLQKPGQSPQLLMY |
| Macaca_mulatta_IGKV2-86*01 | LDWYLQKPGQSPQPLIY |
| Macaca_mulatta_GKV2-90*01 | LDWYLQKPGQSPQLLIY |
| Macaca_mulatta_GKV2-91*01 | LYWYLQKPGQSPQLLMY |
| Macaca_mulatta_IGKV2-99*01 | LDWCLQKPGQSPQLLIY |
| Macaca_mulatta_IGKV2S15*01 | LYWYLQKPGQSPQLLIY |
| Macaca_mulatta_IGKV2S2*01 | LDWYLQKPGQSPRLLIY |
| Macaca_mulatta_IGKV2S20*01 | LEWYLQKPGQSPQPLIY |
| Macaca_mulatta_IGKV2S3*01 | LHWYLQKPGQSPRLLIY |
| Macaca_mulatta_IGKV2S8*01 | LNWLQQKPGQPPRRLIY |
| Macaca_mulatta_IGKV2S9*01 | LNWLQQKPGQPPRRLIY |
| Macaca_mulatta_IGKV3-10*01 | LAWYQQKPGQAPRLLIH |
| Macaca_mulatta_IGKV3-17*01 | LAWYQQKPGQAPRLLIY |
| Macaca_mulatta_IGKV3-24*01 | LAWYQQKPGQAPRLLIY |
| Macaca_mulatta_IGKV3-31*01 | LAWYQQKPGQAPRLLIY |
| Macaca_mulatta_GKV3-35*01 | LAWYQQKPGQAPRLLIY |
| Macaca_mulatta_IGKV3-40*01 | LAWYQQKPGQAPKLLVR |
| Macaca_mulatta_IGKV3-42*01 | LAWYQQKPGQAPRLLIY |
| Macaca_mulatta_IGKV3-53*01 | LAWYQQKPGQAPRLLIY |
| Macaca_mulatta_IGKV3S11*01 | LAWYQQKPGQAPRLLIY |
| Macaca_mulatta_IGKV3S5*01 | LAWYKQKPGQAPRLLIY |
| Macaca_mulatta_IGKV3S9*01 | VAWYQQKPGQAPRLLIY |
| Macaca_mulatta_IGKV4-1*01 | LAWYQQKPGQAPKLLIY |
| Macaca_mulatta_IGKV5-11*01 | MNWYQLEPGEAPKLIIK |
| Macaca_mulatta_IGKV5-5*01 | MNWYQQEPGEAPKLIIK |
| Macaca_mulatta_IGKV6-47*01 | LHWYQQKPDQAPKLFIQ |
| Macaca_mulatta_IGKV6-55*01 | LHWYQQKPDQSPKLLIK |

TABLE 6
FR2 Sequences in IGLV (light chain)
From Representative Species

| | |
|---|---|
| BOVINE | |
| Bos_taurus_IGLV1-12*01 | VSWYQQIPGSAPRTLIY |
| Bos_taurus_IGLV1-21*01 | VGWYQQVPGSGLRTIIY |
| Bos_taurus_IGLV1-26*01 | VSWFQDIPGSAPRTLIY |
| Bos_taurus_IGLV1-31*01 | VSWFQQIPGSAPRTLIY |
| Bos_taurus_IGLV1-43*01 | VSWFQEIPGSAPRTLIY |
| Bos_taurus_IGLV1-47*01 | VSWYQLIPGSAPRTLIY |
| Bos_taurus_IGLV1-55*01 | VGWFQQIPGSAPRTLIY |
| Bos_taurus_IGLV1-63*01 | VSWYQQLPGKAPRLLTY |
| Bos_taurus_IGLV1-64*01 | VGWYQQIPGSAPKTLIY |
| Bos_taurus_IGLV1-73*01 | VSWYQLIPGLAPKTLIY |
| Bos_taurus_IGLV2-6*01 | VGWYQQLPGSAPKTLIY |
| Bos_taurus_IGLV3-2*01 | AHWYQQKPGQAPVLVIY |
| Bos_taurus_IGLV3-3*01 | TQWYQQKPGQAPKLVIY |
| Bos_taurus_IGLV3-4*01 | TQWYQQKPGQGPVRVIY |
| Bos_taurus_IGLV3-5*01 | AHWYQQKPSQAPVLVIY |
| Bos_taurus_IGLV5-72*01 | IYWYQQKAGNPLRYLLR |
| Bos_taurus_IGLV8-38*01 | PSWYQQTPGQAPRNVIY |
| GOAT | |
| Capra_hircus_IGLV1-17*01 | VSWYQQVPGSAPKLLIY |
| Capra_hircus_IGLV1-21*01 | VGWYQQLPGSGLKTVIY |
| Capra_hircus_IGLV1-23*01 | VGWCQQLPGMAPKTLIY |
| Capra_hircus_IGLV1-30*01 | VAWYQQLPGSAPKLLIY |
| Capra_hircus_IGLV1-34*01 | VGWYQQVPGSGLRTLIY |
| Capra_hircus_IGLV1-39*01 | VSWYQQLPGSAPKLLIY |
| Capra_hircus_IGLV1-44*01 | VAWFQQLPGSGLRIVIY |
| Capra_hircus_IGLV1-47*01 | VGWYQQVPGSAPKLLIY |
| Capra_hircus_IGLV1-53*01 | VGWYQQLPGSGLRTIIY |
| Capra_hircus_IGLV1-57*01 | GSWYQQVPGSAPKLLIY |
| Capra_hircus_IGLV1-59*01 | VGWYQQIPGSAPRTLIY |
| Capra_hircus_IGLV1-69*01 | VGWYQLIPGSGLRTIIY |
| Capra_hircus_IGLV1-72*01 | VSWYQQLQGSAPKTLIY |
| Capra_hircus_IGLV2-10*01 | VGWYQQLPGSAPKTLIY |
| Capra_hircus_IGLV2-11*01 | IGWYQQLPGSAPKTLIY |
| Capra_hircus_IGLV3-2*01 | ANWYQLKPGQAPVTVIY |
| Capra_hircus_IGLV3-3*01 | TQWHQQKPGQAPELVIY |
| Capra_hircus_IGLV3-4*01 | TAWYQQKPGQAPVKVIC |
| Capra_hircus_IGLV3-7*01 | AHWYQQKPGQAPVLVIY |
| Capra_hircus_IGLV3-8*01 | VQWHQQKPGQAPVTVIY |
| Capra_hircus_IGLV5-71*01 | IYWYQQKAGSPPRYLLR |
| Capra_hircus_IGLV8-51*01 | PSWYQQTPGQVPRNVIY |
| CAT | |
| Felis_catus_IGLV1-32*01 | VSWYQQLPGTTPKTIIY |
| Felis_catus_IGLV1-36*01 | VNWHQQFPGMAPKTIIY |
| Felis_catus_IGLV1-37*01 | GNWYQQLPGKAPKLLIY |
| Felis_catus_IGLV1-40*01 | VSWYQQVPGMGPKTVIY |
| Felis_catus_IGLV1-42*01 | VSWYQQVPGTAPRLLIY |
| Felis_catus_IGLV1-51*01 | VNWYQQLPGKAPKLLSM |
| Felis_catus_IGLV1-56*01 | VSWYQQLSGTAPKLLIY |
| Felis_catus_IGLV1-61*01 | VNWYQQLPGTTPKTIIL |
| Felis_catus_IGLV1-63*01 | VNWYQQHPGKAPKLLIY |
| Felis_catus_IGLV1-70*01 | VNWYQQFPGKAPKLLIS |
| Felis_catus_IGLV12-26*01 | ASWVQQKPYQRFQGLIG |
| Felis_catus_IGLV2-24*01 | VSWYQQLEGTSPKLLIH |
| Felis_catus_IGLV3-11*01 | TNWHQQKPGQAPIQIIY |
| Felis_catus_IGLV3-13*01 | AYWYQQKSGQAPVLIIY |
| Felis_catus_IGLV3-17*01 | INWYQQKSGQAPVLVIY |
| Felis_catus_IGLV3-2*01 | AYWYQQKPGQAPMLVIY |
| Felis_catus_IGLV3-23*01 | AYWYQQKPGRAPMMIIY |
| Felis_catus_IGLV3-5*01 | VSWYQQKPNQVPLMIIY |
| Felis_catus_IGLV3-6*01 | AYWYQQKSGQAPMLIIY |
| Felis_catus_IGLV3-7*01 | VHWYQQKSGQTPVLIIY |
| Felis_catus_IGLV4-15*01 | VWWYQQRPEKAPRYLMK |
| Felis_catus_IGLV5-30*01 | IFWYQQKPGSLPRYLLT |
| Felis_catus_IGLV5-34*01 | IYWYQQKPGSPPRYLLY |
| Felis_catus_IGLV5-46*01 | IYWYQQKPGSPPRYFLY |
| Felis_catus_IGLV5-48*01 | INWFQQKPGSPPRYLLY |
| Felis_catus_IGLV5-55*01 | ISWFQQKPGSPPRYLLY |
| Felis_catus_IGLV5-68*01 | IYWYQQNPGSPPRYLLY |
| Felis_catus_IGLV5-79*01 | INWYQQNPGNPPRYLLY |
| HUMAN | |
| Homo_sapiens_IGLV1-36*01 | VNWYQQLPGKAPKLLIY |
| Homo_sapiens_IGLV1-40*01 | VHWYQQLPGTAPKLLIY |
| Homo_sapiens_IGLV1-44*01 | VNWYQQLPGTAPKLLIY |
| Homo_sapiens_IGLV1-47*01 | VYWYQQLPGTAPKLLIY |
| Homo_sapiens_IGLV1-51*01 | VSWYQQLPGTAPKLLIY |
| Homo_sapiens_IGLV10-54*01 | AAWLQQHQGHPPKLLSY |
| Homo_sapiens_IGLV2-18*01 | VSWYQQPPGTAPKLMIY |
| Homo_sapiens_IGLV2-8*01 | VSWYQQHPGKAPKLMIY |
| Homo_sapiens_IGLV3-1*01 | ACWYQQKPGQSPVLVIY |
| Homo_sapiens_IGLV3-10*01 | AYWYQQKSGQAPVLVIY |
| Homo_sapiens_IGLV3-16*01 | AYWYQQKPGQFPVLVIY |
| Homo_sapiens_IGLV3-19*01 | ASWYQQKPGQAPVLVIY |
| Homo_sapiens_IGLV3-21*01 | VHWYQQKPGQAPVLVIY |
| Homo_sapiens_IGLV3-22*01 | ADWYQQKPGQAPELVIY |
| Homo_sapiens_IGLV3-25*01 | AYWYQQKPGQAPVLVIY |
| Homo_sapiens_IGLV3-27*01 | ARWFQQKPGQAPVLVIY |
| Homo_sapiens_IGLV3-9*01 | VHWYQQKPGQAPVLVIY |
| Homo_sapiens_IGLV4-3*01 | IEWYQQRPGRSPQYIMK |
| Homo_sapiens_IGLV4-60*01 | IAWHQQQPGKAPRYLMK |
| Homo_sapiens_IGLV4-69*01 | IAWHQQQPEKGPRYLMK |
| Homo_sapiens_IGLV5-37*01 | IYWYQQKPGSPPRYLLY |
| Homo_sapiens_IGLV5-39*01 | IYWYQQKPGSLPRYLLR |
| Homo_sapiens_IGLV5-45*01 | IYWYQQKPGSPPQYLLR |
| Homo_sapiens_IGLV5-52*01 | IRWYQQKPGNPPRYLLY |
| Homo_sapiens_IGLV6-57*01 | VQWYQQRPGSSPTTVIY |
| Homo_sapiens_IGLV7-43*01 | PNWFQQKPGQAPRALIY |
| Homo_sapiens_IGLV7-46*01 | PYWFQQKPGQAPRTLIY |
| Homo_sapiens_IGLV8-61*01 | PSWYQQTPGQAPRTLIY |
| Homo_sapiens_IGLV9-49*01 | VDWYQQRPGKGPRFVMR |
| MOUSE | |
| Mus_musculus_IGLV1*01 | ANWVQEKPDHLFTGLIG |
| Mus_musculus_IGLV2*01 | ANWVQEKPDHLFTGLIG |
| Mus_musculus_IGLV3*01 | IEWYQQQPLKPPKYVME |
| RABBIT | |
| Oryctolagus_cuniculus_ | VYWYRQLPGMSPTLLIY |
| IGLV2S1*01 | |
| Oryctolagus_cuniculus_ | ISWYQQLPGSVPKLLMF |
| IGLV2S2*01 | |
| Oryctolagus_cuniculus_ | VHWYQQKPGLAPGLLIY |
| IGLV3S2*01 | |
| Oryctolagus_cuniculus_ | VHWYQKRPGQAPVLLIY |
| IGLV3S6*01 | |
| Oryctolagus_cuniculus_ | ASWYQQKPGQAPVLLIY |
| IGLV3S7*01 | |
| Oryctolagus_cuniculus_ | VSWYQQKPGLAPVLLIY |
| IGLV3S9*01 | |
| Oryctolagus_cuniculus_ | IEWYQQQQGEAPRYLMQ |
| IGLV4S3*01 | |
| Oryctolagus_cuniculus_ | IDWYQQQQGEAPRYLMQ |
| IGLV4S4*01 | |
| Oryctolagus_cuniculus_ | VLWLQQVPGRPPRYLLT |
| IGLV5S1*01 | |
| Oryctolagus_cuniculus_ | LVWLQQVPGRPPRYLLG |
| IGLV5S10*01 | |
| Oryctolagus_cuniculus_ | LVWLQQVPGRPPRYLLG |
| IGLV5S2*01 | |
| Oryctolagus_cuniculus_ | LVWLQQVPGRPPRYLLT |
| IGLV5S3*01 | |
| Oryctolagus_cuniculus_ | VLWLQQVPGRPPRYLLA |
| IGLV5S5*01 | |
| Oryctolagus_cuniculus_ | VLWLQQVPGRAPRYLLS |
| IGLV5S6*01 | |
| Oryctolagus_cuniculus_ | IGWYQQVPGRPPRYLLT |
| IGLV5S9*01 | |
| Oryctolagus_cuniculus_ | VYWYQQHQGHAPSQLIY |
| IGLV6S1*01 | |
| Oryctolagus_cuniculus_ | VHWYQQHPGKAPTTVIY |
| IGLV6S3*01 | |
| Oryctolagus_cuniculus_ | VHWYQQHPGKAPTTVIY |
| IGLV6S5*01 | |
| SHEEP | |
| Ovis_aries_IGLV1-100*01 | VGWCQQLPGSAPRTLIY |
| Ovis_aries_IGLV1-103*01 | VGWYQQVPGSGLRTLIY |
| Ovis_aries_IGLV1-105*01 | VSWFQQVPGSAPKLLIY |
| Ovis_aries_IGLV1-107*01 | VGWCQQLPGTAPKTPIY |
| Ovis_aries_IGLV1-110*01 | VGWYQQLPGSGLRTIIY |
| Ovis_aries_IGLV1-117*01 | VGWYQQLPGSGLKTIIY |
| Ovis_aries_IGLV1-120*01 | VSWYQQLPGSAPRTLIY |
| Ovis_aries_IGLV1-25*01 | VSWYQQLPGSAPKTLIY |
| Ovis_aries_IGLV1-29*01 | VGWFQQVPGSGLRTVIY |
| Ovis_aries_IGLV1-33*01 | VGWFQQVPGSGLRTVIY |
| Ovis_aries_IGLV2-10*01 | VGWYQQLPGSAPKTLIY |
| Ovis_aries_IGLV2-11*01 | LGWYQQLPGSAPKTLIY |
| Ovis_aries_IGLV2-12*01 | VGWYQQLPGSAPKTLIY |
| Ovis_aries_IGLV3-2*01 | VNWYQLKPGQAPVTVIY |
| Ovis_aries_IGLV3-3*01 | TQWHQQKPGQAPELVIY |
| Ovis_aries_IGLV3-4*01 | TAWYQQKPGQAPVKVIC |
| Ovis_aries_IGLV3-7*01 | AHWYQQKPGQAPVLVIY |
| Ovis_aries_IGLV3-8*01 | VQWHQQKPGQAPVTVIY |
| Ovis_aries_IGLV5-113*01 | IYWYQQKAGSPPRYLLR |
| Ovis_aries_IGLV5-119*01 | IYWYQQKAGSPPRYLLR |
| Ovis_aries_IGLV5-66*01 | IYWYQQKAGSPPRYLLR |
| Ovis_aries_IGLV5-84*01 | IYWYQQKAGSPPRYLLR |
| Ovis_aries_IGLV8-46*01 | PSWYLQTPGQALRNVIY |
| RAT | |
| Rattus_norvegicus_IGLV1S1*01 | ANWIQEKADHLFTGIVG |
| Rattus_norvegicus_IGLV2S1*01 | VSWYQQHLGRPPINVIY |
| Rattus_norvegicus_IGLV3S1*01 | VHWYQQYEGRSPTTMIY |
| Rattus_norvegicus_IGLV3S2*01 | VNWYQQHEGRSPTTMIY |
| Rattus_norvegicus_IGLV3S3*01 | VYWYQQHEGRSPTTMIY |
| Rattus_norvegicus_IGLV3S4*01 | IHWYQQHEGRSPTTMIY |
| Rattus_norvegicus_IGLV3S5*01 | VHWYQQHEGRSPTTMIY |
| Rattus_norvegicus_IGLV4S1*01 | AQWYQQKPDKTIVSVIY |
| PIG | |
| Sus_scrofa_IGLV2-6*01 | VSWYQQHPGLAPKFLIY |
| Sus_scrofa_IGLV3-2*01 | IQWYQQKPGQAPVLFIY |
| Sus_scrofa_IGLV3-3*01 | TQWYQQKPGQAPLLLIY |
| Sus_scrofa_IGLV3-4*01 | AHWYQQKPGQSPMLVIY |
| Sus_scrofa_IGLV3-5*01 | AHWYQQKPGQSPELVIY |
| Sus_scrofa_IGLV5-14*01 | ISWYQRKPGRPPWYLLR |
| Sus_scrofa_IGLV8-10*01 | PSWFQQTPGQPPRLLIY |
| Sus_scrofa_IGLV8-13*01 | PSWFQQTPGQPPRQLIY |
| Sus_scrofa_IGLV8-18*01 | PGWFQQTPGQPPRTVIY |
| Sus_scrofa_IGLV8-19*01 | PGWFQQTPGQPPRTVIY |

TABLE 7
FR2 Sequences From Variable Heavy (VH)
and Variable Light (VL) Domains From
Approved Whole mAbs

| Therapeutic | VH_FR2 | VL_FR2 |
|---|---|---|
| >Adalimumab | MHWVRQAPGKGLEWVSA | LAWYQQKPGKAPKLLIY |
| >Aducanumab | MHWVRQAPGKGLEWVAV | LNWYQQKPGKAPKLLIY |
| >Alemtuzumab | MNWVRQPPGRGLEWIGF | LNWYQQKPGKAPKLLIY |
| >Alirocumab | MNWVRQAPGKGLDWVST | LGWYQQKPGQPPNLLIY |
| >Anifrolumab | IAWVRQMPGKGLESMGI | FAWYQQKPGQAPRLLIY |
| >Ansuvimab | MHWVRQTIDKRLEWVSA | VAWYQQRPGKVPKLLIS |
| >Atezolizumab | IHWVRQAPGKGLEWVAW | VAWYQQKPGKAPKLLIY |
| >Avelumab | MMWVRQAPGKGLEWVSS | VSWYQQHPGKAPKLMIY |
| >Bamlanivimab | ISWVRQAPGQGLEWMGR | LSWYQQKPGKAPKLLIY |
| >Basiliximab | MHWIKQRPGQGLEWIGA | MQWYQQKPGTSPKRWIY |
| >Belimumab | INWVRQAPGQGLEWMGG | ASWYQQKPGQAPVLVIY |
| >Benralizumab | IHWVRQRPGQGLAWMGY | LNWYQQKPGKAPKLLIY |
| >Bevacizumab | MNWVRQAPGKGLEWVGW | LNWYQQKPGKAPKVLIY |
| >Bezlotoxumab | IGWVRQMPGKGLEWMGI | LAWYQQKPGQAPRLLIY |
| >Bimekizumab | MAWVRQAPGKGLEWVAT | MHWYQQKPGKAPKLLIY |
| >Brodalumab | ISWVRQAPGQGLEWMGW | LAWFQQKPGQAPRPLIY |
| >Burosumab | MHWVRQAPGQGLEWMGI | LVWYQQKPGKAPKLLIY |
| >Camrelizumab | MSWVRQAPGKGLEWVAT | LTWYQQKPGKAPKLLIY |
| >Canakinumab | MNWVRQAPGKGLEWVAI | LHWYQQKPDQSPKLLIK |
| >Cemiplimab | MTWVRQAPGKGLEWVSG | LNWYQQKPGKAPNLLIY |
| >Cetuximab | VHWVRQSPGKGLEWLGV | IHWYQQRTNGSPRLLIK |
| >Crizanlizumab | INWVRQAPGKGLEWMGW | MNWYQQKPGKAPKLLIY |
| >Daclizumab | MHWVRQAPGQGLEWIGY | MHWYQQKPGKAPKLLIY |
| >Daratumumab | MSWVRQAPGKGLEWVSA | LAWYQQKPGQAPRLLIY |
| >Denosumab | MSWVRQAPGKGLEWVSG | LAWYQQKPGQAPRLLIY |
| >Dinutuximab | MNWVRQNIGKSLEWIGA | LHWYLQKPGQSPKLLIH |
| >Dostarlimab | MSWVRQAPGKGLEWVST | VAWYQQKPGKAPKLLIY |
| >Dupilumab | MTWVRQAPGKGLEWVSS | LDWYLQKSGQSPQLLIY |
| >Durvalumab | MSWVRQAPGKGLEWVAN | LAWYQQKPGQAPRLLIY |
| >Eculizumab | IQWVRQAPGQGLEWMGE | LNWYQQKPGKAPKLLIY |
| >Efalizumab | MNWVRQAPGKGLEWVGM | LAWYQQKPGKAPKLLIY |
| >Elotuzumab | MSWVRQAPGKGLEWIGE | VAWYQQKPGKVPKLLIY |
| >Emapalumab | MSWVRQAPGKGLEWVSA | VQWYQQRPGSSPTTVIY |
| >Eptinezumab | MNWVRQAPGKGLEWVGV | LAWYQQKPGKVPKQLIY |
| >Erenumab | MHWVRQAPGKGLEWVAV | VSWYQQLPGTAPKLLIY |
| >Etesevimab | MSWVRQAPGKGLEWVSV | LNWYQQKPGKAPKLLIY |
| >Evinacumab | MNWVRQGPGKGLEWVSA | LAWYQQKPGKAPKLLIY |
| >Evolocumab | ISWVRQAPGQGLEWMGW | VSWYQQHPGKAPKLMIY |
| >Fremanezumab | ISWVRQAPGKGLEWVAE | VSWYQQKPGQAPRLLIY |
| >Galcanezumab | MQWVRQAPGQGLEWMGA | LNWYQQKPGKAPKLLIY |
| >Golimumab | MHWVRQAPGNGLEWVAF | LAWYQQKPGQAPRLLIY |
| >Guselkumab | IGWVRQMPGKGLEWMGI | VHWYQQLPGTAPKLLIY |
| >Ibalizumab | IHWVRQKPGQGLDWIGY | LAWYQQKPGQSPKLLIY |
| >Ibritumomab | MHWVKQTPRQGLEWIGA | MHWYQQKPGSSPKPWIY |
| >Inebilizumab | MNWVRQAPGKGLEWVGR | MNWFQQKPDQSPKLLIH |
| >Infliximab | MNWVRQSPEKGLEWVAE | IHWYQQRTNGSPRLLIK |
| >Ipilimumab | MHWVRQAPGKGLEWVTF | LAWYQQKPGQAPRLLIY |
| >Isatuximab | MQWVKQRPGQGLEWIGT | VAWYQQKPGQSPRRLIY |
| >Itolizumab | MSWVRQAPGKRLEWVAT | LTWYQQKPGKAPKTLIY |
| >Ixekizumab | IHWVRQAPGQGLEWMGV | LHWYLQKPGQSPQLLIY |
| >Lanadelumab | MMWVRQAPGKGLEWVSG | LAWYQQKPGKAPKLLIY |
| >Levilimab | MSWVRQAPGKGLEWVSG | LNWYQQKPGQAPQLLIY |
| >Margetuximab | IHWVKQRPEQGLEWIGR | VAWYQQKPGHSPKLLIY |
| >Masavibart | MYWVRQAPGKGLEWVAV | VSWYQQHPGKAPKLMIY |
| >Mepolizumab | VHWVRQPPGKGLEWLGV | LAWYQQKPGQPPKLLIY |
| >Mogamulizumab | MSWVRQAPGKGLEWVAT | LEWYLQKPGQSPQLLIY |
| >Muromonab | MHWVKQRPGQGLEWIGY | MNWYQQKSGTSPKRWIY |
| >Natalizumab | IHWVRQAPGQRLEWMGR | MAWYQQTPGKAPRLLIH |
| >Naxitamab | VHWVRQPPGKGLEWLGV | VTWYQQKPGQAPRLLIY |
| >Necitumumab | WSWIRQPPGKGLEWIGY | LAWYQQKPGQAPRLLIY |
| >Nepuvibart | MSWIRQAPGKGLEWVSY | LNWYQQKPGKAPKLLIY |
| >Netakimab | MGWLRQAPGKGTEFVAA | LAWYQQKPGQAPRLLIY |
| >Nimotuzumab | IYWVRQAPGQGLEWIGG | LDWYQQTPGKAPKLLIY |
| >Nivolumab | MHWVRQAPGKGLEWVAV | LAWYQQKPGQAPRLLIY |
| >Obiltoxaximab | MNWVRQAPGQGLEWIGR | LNWYQQKPGKAVKLLIY |
| >Obinutuzumab | INWVRQAPGQGLEWMGR | LYWYLQKPGQSPQLLIY |
| >Ocrelizumab | MHWVRQAPGKGLEWVGA | MHWYQQKPGKAPKPLIY |
| >Odesivimab | MHWVRQATGKGLEWVSA | LAWYQQKPGQPPKLLIY |
| >Ofatumumab | MHWVRQAPGKGLEWVST | LAWYQQKPGQAPRLLIY |
| >Olaratumab | WGWLRQSPGKGLEWIGS | LAWYQQKPGQAPRLLIY |
| >Omalizumab | WNWIRQAPGKGLEWVAS | MNWYQQKPGKAPKLLIY |
| >Palivizumab | VGWIRQPPGKALEWLAD | MHWYQQKPGKAPKLLIY |
| >Panitumumab | WTWIRQSPGKGLEWIGH | LNWYQQKPGKAPKLLIY |
| >Pembrolizumab | MYWVRQAPGQGLEWMGG | LHWYQQKPGQAPRLLIY |
| >Penpulimab | MSWVRQAPGKGLDWVAT | LSWFQQKPGKSPKTLIY |
| >Pertuzumab | MDWVRQAPGKGLEWVAD | VAWYQQKPGKAPKLLIY |
| >Racotumomab | INWVRQRPEQGLEWIGW | LNWYQQKPDGTVKLLIY |
| >Ramucirumab | MNWVRQAPGKGLEWVSS | LGWYQQKPGKAPKLLIY |
| >Ravulizumab | IQWVRQAPGQGLEWMGE | LNWYQQKPGKAPKLLIY |
| >Regdanvimab | VGWIRQPPGKALEWLAL | VSWYQQLPGTAPKLLIY |
| >Reslizumab | VNWIRQAPGKGLEWVGL | LAWYQQKPGKAPKLLIY |
| >Risankizumab | IHWMRQAPGQGLEWIGY | VAWYQQKPGKVPKLLIY |
| >Rituximab | MHWVKQTPGRGLEWIGA | IHWFQQKPGSSPKPWIY |
| >Romosozumab | MHWVRQAPGQGLEWMGE | LNWYQQKPGKAPKLLIY |
| >Sarilumab | MHWVRQAPGKGLEWVSG | LAWYQQKPGKAPKLLIY |
| >Satralizumab | WSWVRQPPGEGLEWIGF | LNWYQQKPGKAPELLIY |
| >Secukinumab | MNWVRQAPGKGLEWVAA | LAWYQQKPGQAPRLLIY |
| >Siltuximab | MSWFRQSPEKRLEWVAE | MYWYQQKPGSSPRLLIY |
| >Sintilimab | ISWVRQAPGQGLEWMGL | LAWYQQKPGKAPKLLIS |
| >Sotrovimab | ISWVRQAPGQGLEWMGW | LAWYQQKPGQAPRLLIY |
| >Tafasitamab | MHWVRQAPGKGLEWIGY | LYWFQQKPGQSPQLLIY |
| >Teprotumumab | MHWVRQAPGKGLEWVAI | LAWYQQKPGQAPRLLIY |
| >Tildrakizumab | MTWVRQAPGQGLEWMGQ | LAWYQQKPGKAPKLLIY |
| >Tislelizumab | VHWIRQPPGKGLEWIGV | VAWYQQKPGQPPKLLIN |
| >Tocilizumab | WSWVRQPPGRGLEWIGY | LNWYQQKPGKAPKLLIY |
| >Toripalimab | MHWVRQAPIHGLEWIGV | LEWYLQKPGQSPQLLIY |
| >Tralokinumab | LSWVRQAPGQGLEWMGW | VHWYQQKPGQAPVLVIY |
| >Trastuzumab | IHWVRQAPGKGLEWVAR | VAWYQQKPGKAPKLLIY |
| >Ustekinumab | LGWVRQMPGKGLDWIGI | LAWYQQKPEKAPKSLIY |
| >Vedolizumab | MHWVRQAPGQRLEWIGE | LSWYLQKPGQSPQLLIY |
| >Zimberelimab | WVWIRQPPGKGLEWIGS | VSWYQQHPGKAPELMIY |

TABLE 8
FR2 Sequences from Variable
Heavy (VH) and Variable Light (VL) Domains
From Approved mAb Conjugates

| Therapeutic | Type | VH_FR2 | VL_FR2 |
|---|---|---|---|
| >Belantamab | mAb-ADC | MHWVRQAPGQGLEWMGA | LNWYQQKPGKAPKLLIY |
| >Brentuximab | mAb-ADC | ITWVKQKPGQGLEWIGW | MNWYQQKPGQPPKVLIY |
| >Disitamab | mAb-ADC | IHWVQQAPGKGLEWMGR | VAWYQQKPGKAPKLLIY |
| >Enfortumab | mAb-ADC | MNWVRQAPGKGLEWVSY | LAWYQQKPGKAPKFLIY |
| >Gemtuzumab | mAb-ADC | IHWVRQAPGQSLEWIGY | LTWFQQKPGKAPKLLMY |
| >Inotuzumab | mAb-ADC | IHWVRQAPGQGLEWIGG | LSWYLHKPGKAPQLLIY |
| >Loncastuximab | mAb-ADC | MHWVKQAPGQGLEWIGE | MHWYQQKPGTSPRRWIY |
| >Polatuzumab | mAb-ADC | IEWVRQAPGKGLEWIGE | LNWYQQKPGKAPKLLIY |
| >Sacituzumab | mAb-ADC | MNWVKQAPGQGLKWMGW | VAWYQQKPGKAPKLLIY |
| >Bintrafusp | mAb fusion | MMWVRQAPGKGLEWVSS | VSWYQQHPGKAPKLMIY |
| >Pabinafusp | mAb fusion | LGWVRQMPGKGLEWMGD | LHWYLQKPGQSPQLLIY |
| >Derlotuximab | radiolabel | VRWIRQPPGKGLEWLGV | LHWYQQKSGASPKLWIY |

TABLE 9
FR2 Sequences From Variable
Heavy (VH) and Variable Light (VL) Domains From
Approved Alternative mAb Formats

| Therapeutic | Format | FR2_VH | FR2_VL |
|---|---|---|---|
| Abciximab | Fab | MHWVKQRPG | LHWFQQTSH |
| | | QGLEWIGA | ESPRLLIK |
| Certolizumab | Fab | MNWVRQAPG | VAWYQQKPG |
| | | KGLEWMGW | KAPKALIY |
| Idarucizumab | Fab | VDWIRQPPG | LYWFLQRPG |
| | | KGLEWIGV | QSPRRLIY |
| Ranibizumab | Fab | MNWVRQAPG | LNWQQKPGK |
| | | KGLEWVGW | APKVLIY |
| Moxetumomab | Fv | MSWVRQTPE | LNWQQKPDG |
| | Fusion | KCLEWVAY | TVKLLIY |
| Brolucizumab | scFv | MTWVRQAPG | LAWYQQKPG |
| | | KGLEWVGF | KAPKLLIY |

TABLE 10
FR2 Sequences From Variable Heavy (VH)
and Variable Light (VL) Domains
From Approved Bispecific Antibody Formats

| Therapeutic | Type | | |
|---|---|---|---|
| | | FR2-VH1 | FR2-VL1 |
| Amivantamab | Bispec- | MHWVRQAPG | LNWYQQKPG |
| | mAb | KGLEWVAV | KAPKLLIY |
| Emicizumab | Bispec- | MDWVRQAPG | LAWYQQKPG |
| | mAb | QGLEWMGD | QAPELLIY |
| Blinatumomab | BITE | MNWVKQRPG | LNWYQQIPG |
| | | QGLEWIGQ | QPPKLLI |
| | | FR2-VH2 | FR2-VL2 |
| Amivantamab | Bispec- | ISWVRQAPG | LAWFQHKPG |
| | mAb | HGLEWMGW | KAPKLLIY |
| Emicizumab | Bispec- | IQWVRQAPG | LAWYQQKPG |
| | mAb | KGLEWVSS | QAPELLLIY |
| Blinatumomab | BITE | MHWVKQRPG | MNWYQQKSG |
| | | QGLEWIGY | TSPKRWIY |

TABLE 11
Sequences of Exemplary Antigen-binding Proteins
SEQ ID NO: 1
Amino acid sequence of a Gly-Ser Linker
GSSG
SEQ ID NO : 2
Amino acid sequence of a Gly-Ser Linker
GSSGSSGS
SEQ ID NO: 3
Amino acid sequence of UL -CDR3 from NC-Cow1 (PMID:
28726771)
TNKKECPEDYTYNPRCPQQYGWSDCDCMGDRFGGYCRQDGCSNYIHRST
SEQ ID NO : 4
Amino acid sequence of the VACV (Vaccinia Virus) A 56R protein
AFFMTSTTNDTDKVDYEEYSTELIVNTDSESTIDIILSGSTHSPETSSKKPDYIDNSNCS
SVFEIATPEPITDNVEDHTDTVTYTSDSINTVSASSGESTTDETPEPITDKEDHTVTDTV
SYTTVSTSSGIVTTKSTTDDADLYDTYNDNDTVPPTTVGGSTTSISNYKTKDFVEIFGI
TALIILSAVAIFCITYYIYNKRSRKYKTENKV
SEQ ID NO: 5
Amino acid sequence of VHH -72.18 | SAR S-COV-2 spike VHH -
His8 tag-HA tag
METDTLLLWVLLLWVPGSTGDQVQLQESGGGLVQAGGSLRLSCAASGRTFSEYAM
GWFRQAPGKEREFVATISWSGGSTYYTDSVKGRFTISRDNAKNTVYLQMNSLKPDD
TAVYYCAAAGLGTVVSEWDYDYDYWGQGTQVTVSSGSSGSGSGSEHHHHHHHHS
GY PY DVPDY A
Note: The underlined indicates VHH-72.
SEQ ID NO: 6
Amino acid sequence of ODIN-1 | VHH-72.18 with UL -CDR 3 from
NC-Cow1 bovine IgG in FR2 loop
METDTLLLWVLLLWVPGSTGDQVQLQESGGGLVQAGGSLRLSCAASGRTFSEYAM
GWFRQTNKKECPEDYTYNPRCPQQYGWSDCDCMGDRFGGYCRQDGCSNYIHR
STREFVATISWSGGSTYYTDSVKGRFTISRDNAKNTVYLQMNSLKPDDTAVYYCAA
AGLGTVVSEWDYDYDYWGOGTOVTVSSGSSGSGSGSEHHHHHHHHSGYPYDVPD
YA
Note: The underlined indicates VHH-72; the bolded font indicates UL -CDR 3 from NC-
Cow1 (PMID: 28726771).
SEQ ID NO : 7
Amino acid sequence of ODIN-2 | VHH-72.18 with a 4aa Gly-Ser
linker in in FR 2 loop
METDTLLLWVLLLWVPGSTGDQVQLQESGGGLVQAGGSLRLSCAASGRTFSEYAM
GWFRQGSSGEREFVATISWSGGSTYYTDSVKGRFTISRDNAKNTVYLQMNSLKPDD
TAVYYCAAAGLGTVVSEWDYDYDYWGQGTOVTVSSGSSGSGSGSEHHHHHHHHS
GYPYDCPDYA
Note: The underlined indicates VHH-72; the bolded font indicates the GSSG linker insertion.
SEQ ID NO: 8
Amino acid sequence of ODIN-3 | VHH-72.18 with an 8aa Gly-Ser
linker in in FR2 loop
METDTLLLWVLLLWVPGSTGDQVQLQESGGGLVQAGGSLRLSCAASGRTFSEYAM
GWFRQGSSGSSGSEREFVATISWSGGSTYYTDSVKGRFTISRDNAKNTVYLQMNSLK
PDDTAVYYCAAAGLGTVVSEWDYDYDYWGOGTOVTVSSGSSGSGSGSEHHHHHH
HHSGYPYDVPDYA
Note: The underlined indicates VHH-72; the bolded font indicates the GSSGSSGS linker
insertion.
SEQ ID NO: 9
Amino acid sequence of the VHH-72-A 56R fusion | VHH-72 fused
to vaccinia virus (VACV) A56R for rVACV display
METDTLLLWVLLLWVPGSTGDQVQLQESGGGLVQAGGSLRLSCAASGRTFSEYAM
GWFRQAPGKEREFVATISWSGGSTYYTDSVK GRFTISRDNAKNTVYLQMNSLKPDD
TAVYYCAAAGLGTVVSEWDYDYDYWGQGTQVTVSSGSSGSGSGSEHHHHHHHHS
GYPYDVPDYAGSSSGGSSGEGS

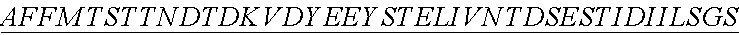

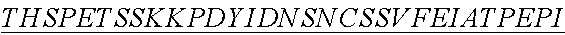

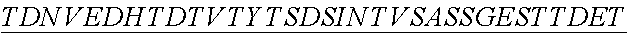

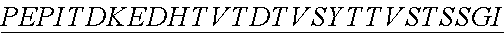

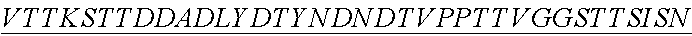

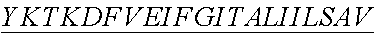

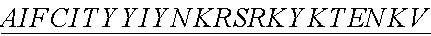

Note: Single underline indicates VHH-72; and wavy underline indicates VACV A 56R.
SEQ ID NO: 10
Amino acid sequence of the ODIN-1-A56R fusion | ODIN-1 fused
to vaccinia virus (VACV) A56R for rVACV display
METDTLLLWVLLLWVPGSTGDQVQLQESGGGLVQAGGSLRLSCAASGRTFSEYAM
GWFRQTNKKECPEDYTYNPRCPQQYGWSDCDCMGDRFGGYCRQDGCSNYIHR
STREFVATISWSGGSTYYTDSVKGRFTISRDNAKNTVYLQMNSLKPDDTAVYYCAA
AGLGTVVSEWDYDYDYWGQGTQVTVSSGSSGSGSGSEHHHHHHHHSGYPYDVPD
YAGSSSGGSSGEGS

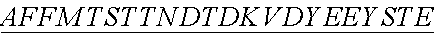

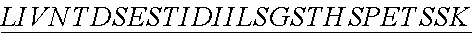

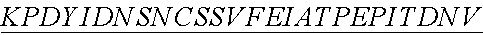

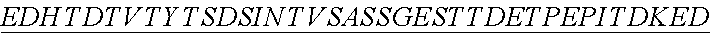

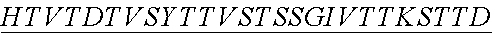

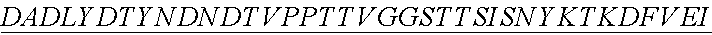

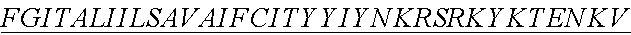

Note: Single underline indicates VHH-72; the bolded font indicates UL -CDR 3 from NC-
Cow1 (PM ID: 28726771); and wavy underline indicates VACV A 56R.
SEQ ID NO: 11
Amino acid sequence of the ODIN-2-A56R fusion | ODIN-2 fused
METDTLLLWVLLLWVPGSTGDQVQLQESGGGLVQAGGSLRLSCAASGRTFSEYAM
GWFRQGSSGEREFVATISWSGGSTYYTDSVK GRFTISRDNAKNTVY LQMNSLKPDD
TAVYYCAAAGLGTVVSEWDYDYDYWGQGTQVTVSSGSSGSGSGSEHHHHHHHHS

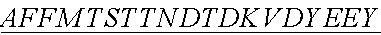

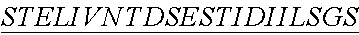

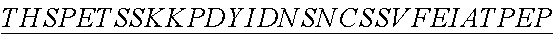

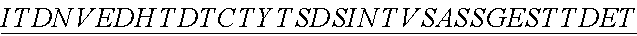

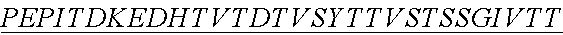

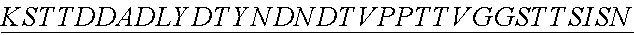

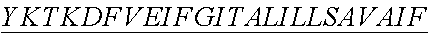

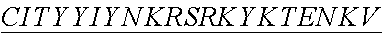

Note: Single underline indicates VHH-72; the bolded font indicates the GSSG linker
insertion; and wavy underline indicates VACV A56R.
SEQ ID NO: 12
Amino acid sequence of the ODIN-3-A56R fusion | ODIN-3 fused
to vaccinia virus (VACV) A56R for rVACV display
METDTLLLWVLLLWVPGSTGDQVQLQESGGGLVQAGGSLRLSCAASGRTFSEYAM
GWFRQGSSGSSGSEREFVATISWSGGSTYYTDSVKGRFTISRDNAKNTVYLQMNSLK
PDDTAVYYCAAAGLGTVVSEWDYDYDYWGQGTQVTVSS
HHSGYPYDVPDYAGSSSGGSSGEGS

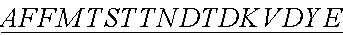

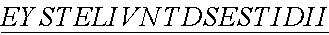

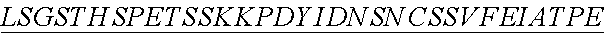

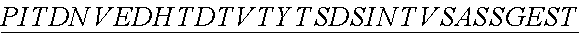

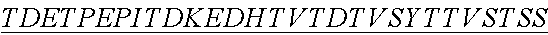

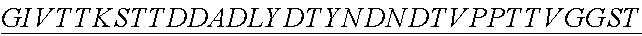

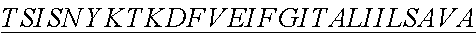

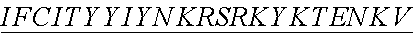

Note: Single underline indicates VHH-72; the bolded font indicates the GSSGSSGS linker
insertion; and wavy underline indicates VACV A56R.
SEQ ID NO: 13
Amino acid sequence of ODIN-4 | VHH-72.18 with UL -CDR3 from
NC-Cow1 bovine IgG in FR 2 loop version 2
METDTLLLWVLLLWVPGSTGDQVQLQESGGGLVQAGGSLRLSCAASGRTFSEYAM
GWFRTNKKECPEDYTYNPRCPQQYGWSDCDCMGDRFGGYCRQDGCSNYIHRS
TREFVATISWSGGSTYYTDSVKGRFTISRDNAKNTVYLQMNSLKPDDTAVYYCAAA
GLGTVVSEWDYDYDYWGQGTQVTVSSGSSGSGSGSEHHHHHHHHSGY PYDVPDYA
Note: The underlined indicates VHH-72; the bolded font indicates UL-CDR 3 from NC-
Cow1 (PM ID: 28726771).
SEQ ID NO: 14
Amino acid sequence of VHH 72.58 | SAR S-COV-2 spike VHH -
Double strep tag
METDTLLLWVLLLWVPGSTGDQVQLQESGGGLVQAGGSLRLSCAASGRTFSEYAM
GWFRQAPGKEREFVATISWSGGSTYYTDSVKGRFTISRDNAKNTVYLQMNSLKPDD
TAVYYCAAAGLGTVVSEWDYDYDYWGQGTQVTVSSGSSGSGSGSESAWSHPQFEK
GGGSGGGSGGSSAWSHPQFEK
Note: The underlined indicates VHH-72.
SEQ ID NO : 15
Amino acid sequence of ODIN-5 | VHH-72.58 with UL -CDR 3 from
NC -Cow1 bovine IgG in FR 2 loop
METDTLLLWVLLLWVPGSTGDQVQLQESGGGLVQAGGSLRLSCAASGRTFSEYAM
GWFRQTNKKECPEDYTYNPRCPQQYGWSDCDCMGDRFGGYCRQDGCSNYIHR
STREFVATISWSGGSTYYTDSVKGRFTISRDNAKNTVYLQMNSLKPDDTAVYYCAA
AGLGTVVSEWDYDYDYWGOGTQVTVSSGSSGSGSGSESAWSHPQFEKGGGSGGGS
GGSSAWSHPQFEK
Note: The underlined indicates VHH-72; the bolded font indicates UL -CDR 3 from NC-
Cow1 (PMID: 28726771).
SEQ ID NO: 16
Amino acid sequence of ODIN-5 Fc V1 | ODIN-5 VHH and UL -
CDR 3 sequence with C-terminal Fc fusion version 1
METDTLLLWVLLLWVPGSTGEVQLQESGGGLVQAGGSLRLSCAASGRTFSEYAMG
WFRQTNKKECPEDYTYNPRCPQQYGWSDCDCMGDRFGGYCRQDGCSNYIHRS
TREFVATISWSGGSTYYTDSVKGRFTISRDNAKNTVYLQMNSLKPDDTAVYYCAAA
GLGTVVSEWDYDYDYWGQGTQVTVSS

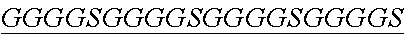

DKTHTCPPC
PAPELLGGPSVFLFPPKPKDTLMISRTPEVTCVVVDVSHEDPEVKFNWYVDGVEVHN
AKTKPREEQYNSTYRVVSVLTVLHODWLNGKEYKCKVSNKALPAPIEKTISKAKGQ
PREPQVYTLPPSRDELTKNOVSLTCLVKGFYPSDIAVEWESNGOPENNYKTTPPVLDS
DGSFFLYSKLTVDKSRWQQGNVFSCSVMHEALHNHYTOKSLSLSPG
Note: Single underline indicates VHH-72; the bolded font indicates UL-CDR 3 from NC-
Cow1 (PM ID: 28726771); the wavy underline indicates a glycine-serine linker; double
underline indicates the amino acid sequence from human IgG Fc.
SEQ ID NO: 17
Amino acid sequence of ODIN-5 Fc V2 | ODIN-5 VHH and UL -
CDR 3 sequence with C-terminal Fc fusion version 2
METDTLLLWVLLLWVPGSTGEVQLQESGGGLVQAGGSLRLSCAASGRTFSEYAMG
WFRQTNKKECPEDYTYNPRCPQQYGWSDCDCMGDRFGGYCRQDGCSNYIHRS
TREFVATISWSGGSTYYTDSVKGRFTISRDNAKNTVYLQMNSLKPDDTAVYYCAAA
GLGTVVSEWDYDYDYWGQGTQVTVSS EPKSSDKTHTCPPCPAPELLGGPSVFLFPPK
PKDTLMISRTPEVTCVVVDVSHEDPEVKENWYVDGVEVHNAKTKPREEQYNSTYRV
VSVLTVLHODWLNGKEYKCKVSNKALPAPIEKTISKAKGQPREPQVYTLPPSRDELT
KNQVSLTCLVKGFY PSDIAVEWESNGQPENNYKTTPPVLDSDGSFFLYSKLTVDKSR
WQQGNVFSCSVM HEALHNHYTOKSLSLSPG
Note: Single underline indicates VHH-72; the bolded font indicates UL-CDR 3 from NC-
Cow1 (PMID: 28726771); double underline indicates the amino acid sequence from human
lgG Fc
SEQ ID NO: 18
Amino acid sequence of UL -CDR 3 from EGFR bovine lgG 60H 05
(PMID: 34759924)
NKLKSCPDGYTSGVECRFRGYTCANDGCWRVCSFTTCSGWMPASDTYR
SEQ ID NO: 19
Amino acid sequence of VHH 55.84 | MERS-COV spike VHH-
Double Strep tag
METDTLLLWVLLLWVPGSTGQVQLQESGGGSVQAGGSLRLSCVASGSIFSINAMDW
YRQAPGKQRELVAGITSGGSTNYGDFVKGRFTISRDNAKNTVYLOMDSLKPEDTAV
YYCAAEVGGWGPPRPDYWGHGTQVTVSSGSSGSGSGSESAWSHPQFEKGGGSGGG
SGGSSAWSHPQFEK
Note: The underlined indicates VHH55 (PM ID: 34759924).
SEQ ID NO: 20
Amino acid sequence of ODIN-6 | VHH 55.84 with UL -CDR 3 from
NC-Cow1 bovine IgG in FR 2 loop
METDTLLLWVLLLWVPGSTGQVQLQESGGGSVQAGGSLRLSCVASGSIFSINAMDW
YRQTNKKECPEDYTYNPRCPQQYGWSDCDCMGDRFGGYCRQDGCSNYIHRST
RELVAGITSGGSTNY GDFVKGRFTISRDNAKNTVYLOMDSLKPEDTAVYYCAAEVG
GWGPPRPDYWGHGTQVTVSSGSSGSGSGSESAWSHPQFEKGGGSGGGSGGSSAWS
HPQFEK
Note: The underlined indicates VHH55, the bolded font indicates UL-CDR3 from NC-Cow1.
SEQ ID NO : 21
Amino acid sequence of ODIN-7 | VHH 55.84 with UL -CDR3 from
60H 05 bovine IgG in FR 2 loop
METDTLLLWVLLLWVPGSTGQVQLQESGGGSVQAGGSLRLSCVASGSIFSINAMDW
YRQTNKLKSCPDGYTSGVECRFRGYTCANDGCWRVCSFTTCSGWM PASDTYRR
ELVAGITSGGSTNYGDFVKGRFTISRDNAKNTVYLOMDSLKPEDTAVYYCAAEVGG
WGPPRPDYWGHGTQVTVSSGSSGSGSGSESAWSHPQFEKGGGSGGGSGGSSAWSHP
QFEK
Note: The underlined indicates VHH55; the bolded font indicates UL -CDR3 from 60H 05.
SEQ ID NO: 22
Amino acid sequence of ODIN-8 | VHH-72.58 with UL -CDR 3 from
60H 05 bovine IgG in FR 2 loop
METDTLLLWVLLLWVPGSTGDQVQLQESGGGLVQAGGSLRLSCAASGRTFSEYAM
GWFRQNKLKSCPDGYTSGVECRFRGYTCANDGCWRVCSFTTCSGWMPASDTY
RREFVATISWSGGSTYYTDSVKGRFTISRDNAKNTVY LQMNSLKPDDTAVYY CAAA
GLGTVVSEWDYDYDYWGQGTQVTVSSGSSGSGSGSESAWSHPQFEKGGGSGGGSG
GSSAWSHPQFEK
Note: The underlined indicates VHH-72; the bolded font indicates UL -CDR 3 from 60H 05.
SEQ ID NO: 23
Amino acid sequence of VHHD5.77 | LASV glycoprotein VHH-
Double Strep tag
METDTLLLWVLLLWVPGSTGAWQLVESGGGSVQPGGSLTLTCQASKSTESTSGMR
WERQAQGKGVEFVADISSDSTRKWYSDSVKGRFTISRSNWWRTVTLQMNDLKPED
TARYYCKDLESHHLRGQGTQVTVSSSGSSGSGSGSESAWSHPQFEKGGGSGGGSGG
SSAWSHPQFEK
Note: The underlined indicates VHHD5.77.
SEQ ID NO: 24
Amino acid sequence of ODIN-9 | VHHD5.77 with UL -CDR 3 from
NC -Cow1 bovine IgG in FR 2 loop
METDTLLLWVLLLWVPGSTGAWQLVESGGGSVQPGGSLTLTCQASKSTESTSGMR
WERQTNKKECPEDYTYNPRCPQQYGWSDCDCMGDRFGGYCRQDGCSNYIHRS
TVEFVADISSDSTRKWYSDSVKGRFTISRSNWWRTVTLQMNDLKPEDTARYYCKDL
ESHHLRGQGTQVTVSSSGSSGSGSGSESAWSHPQFEKGGGSGGGSGGSSAWSHPQFE
K
Note: The underlined indicates VHHD 5.77; the bolded font indicates UL-CDR 3 from NC-
Cow1.
SEQ ID NO: 25
Amino acid sequence of NC-Cow1 LC | NC -Cow1 variable light
sequence fused to human lambda constant domain
MGDNDIHFAFLSTGAHSSYELTQPSSVSGSLGORVSVTCSGSSSNVGNGYVSWYQLI
PGSAPRTIIYGDTSRASGVPERFSGSRSGNTATLTISSLQAEDEADFFCASPDDSSSNAV
FGSGTTLTVLRTGQPKAAPSVTLFPPSSEELQANKATLVCLISDFYPGAVTVAWK
ADSSPVKAGVETTTPSKQSNNKYAASSYLSLTPEQWKSHRSYSCQVTHEGSTVE
KTVAPTECS
Note: The underlined indicates NC-Cow 1 variable light region; the bolded font indicates
human lambda constant domain.
SEQ ID NO : 26
Amino acid sequence of NC-Cow1 HC INC -Cow1 variable heavy
sequence fused to human IgG 1 constant domain
MGDNDIHFAFLSTGAHSQVQLRESGPSLMKPSQTLSLTCTVSGSSLNDKSVGWVRQA
PGKALQWLGSVDTSGNTDYNPGLKSRLSITKDNSKSRISLTVTGMTTEDSATYYCITA
HQKTNKKECPEDYTYNPRCPQQYGWSDCDCMGDRFGGYCRQDGCSNYIHRSTYEW
YVSAWGQGLLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSV
MHEALHNHYTQKSLSLSPGK
Note: The underlined indicates NC-Cow 1 variable heavy region; the bolded font indicates
human IgG constant domain.
SEQ ID NO : 27
Amino acid sequence of ADI-15878 L C | K appa light chain of
antibody ADI-15878
MGDNDIHFA FLSTGAHSDIQMTOSPSTLSASVGDRVTITCRASQSISSWLAWYQQKP
GEAPKLLISDASSLESGVPSRFSGSGSGTEFTLTISSLOPDDFATYYCOQYYSSPTFGG
GTKVEIKRTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPVTKSFN
RGEC
Note: The underlined indicates A DI-15878 variable light region; the bolded font indicates
human kappa light chain constant domain.
SEQ ID NO: 28
Amino acid sequence of ADI-15878 L C | Heavy chain of antibody
ADI-15878
MGDNDIHFAFLSTGAHSEVQLVESGGGLVOPGGSLRVSCAASGFTFSSYAMSWVRQ
APGKGLEWVSAISGLGGSTYYADSVKGRFTISRDNSKNTLYLQMNSLRAEDTAVYY
CAKDHRVWAAGYHFDYWGQGTLVTVSSASTKGPSVFPLAPSSKSTSGGTAALGC
LVKDYFPEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKS
RWQQGNVFSCSVMHEALHNHYTQKSLSLSPGK
Note: The underlined indicates A DI-15878 variable heavy region; the bolded font indicates
human IgG constant domain.
SEQ ID NO: 29
Amino acid sequence of 15878 scFv | Single chain variable domain
(VL + VH) of ADI-15878
METDTLLLWVLLLWVPGSTGDIQMTQSPSTLSASVGDRVTITCRASQSISSWLAWYQ
QKPGEAPKLLISDASSLESGVPSRFSGSGSGTEFTLTISSLOPDDFATYYCOQYYSSPTF
GGGTKVEIKGGGGSGGGGSGGGGSEVQLVESGGGLVQPGGSLRVSCAASGFTFSS
YAMSWVRQAPGKGLEWVSAISGLGGSTYYADSVKGRFTISRDNSKNTLYLQMN
SLRAEDTAVYYCAKDHRVWAAGYHFDYWGQGTLVTVSSGSSGSGSGSESAWSH
PQFEKGGGSGGGSGGSSAWSHPQFEK
Note: The underlined indicates A DI-15878 variable light region; the bolded font indicates
ADI-15878 variable heavy region.
SEQ ID NO: 30
Amino acid sequence of 15878 scFv Fc V1 | Single chain variable
domain (VL + VH) of ADI-15878 fused to human IgG 1 constant domain
METDTLLLWVLLLWVPGSTGDIQMTQSPSTLSASVGDRVTITCRASQSISSWLAWYQ
QKPGEAPKLLISDASSLESGVPSRFSGSGSGTEFTLTISSLOPDDFATYYCOQYYSSPTF
GGGTKVEIKGGGGSGGGGSGGGGSEVQLVESGGGLVQPGGSLRVSCAASGFTFSS
YAMSWVRQAPGKGLEWVSAISGLGGSTYYADSVKGRFTISRDNSKNTLYLQMN
SLRAEDTAVYYCAKDHRVWAAGYHFDYWGQGTLVTVSSGGGGSGGGGSGGGG
SGGGGSDKTHTCPPCPAPELLGGPSVFLFPPKPKDTLMISRTPEVTCVVVDVSHEDPE
VKFNWYVDGVEVHNAKTKPREEQYNSTY RVVSVLTVLHODWLNGKEYKCKVSNK
ALPAPIEKTISKAKGQPREPQVYTLPPSRDELTKNQVSLTCLVKGFY PSDIAVEWESN
GQPENNY KTTPPVLDSDGSFFLY SKLTVDK SRWQQGNVFSCSVMHEALHNHYTOKS
LSLSPG
Note: The underlined indicates A DI-15878 variable light region; the bolded font indicates
ADI-15878 variable heavy region; double underline indicates the amino acid sequence from
human IgG Fc.
SEQ ID NO: 31
Amino acid sequence of ODIN-10 | 15878 scF v with UL -CDR3
from NC -Cow1 bovine IgG in Vh FR 2 loop
METDTLLLWVLLLWVPGSTGDIQMTQSPSTLSASVGDRVTITCRASQSISSWLAWYQ
QKPGEAPKLLISDASSLESGVPSRFSGSGSGTEFTLTISSLQPDDFATYYCOQYYSSPTF
GGGTKVEIKGGGGSGGGGSGGGGSEVQLVESGGGLVQPGGSLRVSCAASGFTFSS
YAMSWVRQTNKKECPEDYTYNPRCPQQYGWSDCDCMGDRFGGYCRQDGCSNYIH
RSTLEWVSAISGLGGSTYYADSVKGRFTISRDNSKNTLYLQMNSLRAEDTAVYY
CAKDHRVWAAGYHFDYWGQGTLVTVSSGSSGSGSGSESAWSHPQFEKGGGSGG
GSGGSSAWSHPQFEK
Note: The underlined indicates A DI-15878 variable light region; the bolded font indicates
ADI-15878 variable heavy region; double underline indicates the amino acid sequence from
human IgG Fc.
SEQ ID NO: 32
Amino acid sequence of ODIN-11 | 15878 scF v with UL-CDR3
from NC -Cow1 bovine IgG in Vk FR 2 loop
METDTLLLWVLLLWVPGSTGDIQMTOSPSTLSASVGDRVTITCRASQSISSWLAWYQ
QTNKKECPEDYTYNPRCPQQYGWSDCDCMGDRFGGYCRQDGCSNYIHRST
VKLLIS
DASSLESGVPSRFSGSGSGTEFTLTISSLOPDDFATYYCOQYYSSPTFGGGTKVEIKGG
GGSGGGGSGGGGSEVOLVESGGGLVQPGGSLRVSCAASGFTFSSYAMSWVRQAP
GKGLEWVSAISGLGGSTYYADSVKGRFTISRDNSKNTLYLQMNSLRAEDTAVYY
CAKDHRVWAAGYHFDYWGQGTLVTVSSGSSGSGSGSESAWSHPQFEKGGGSGG
GSGGSSAWSHPQFEK
Note: The underlined indicates A DI-15878 variable light region; the bolded font indicates
ADI-15878 variable heavy region; double underline indicates UL-CDR3 from NC-Cow1.
SEQ ID NO : 33
Amino acid sequence of ODIN-10 Fc V1 | ODIN-10 fused to human
IgG 1 constant domain
METDTLLLWVLLLWVPGSTGDIQMTQSPSTLSASVGDRVTITCRASQSISSWLAWYQ
QKPGEAPKLLISDASSLESGVPSRFSGSGSGTEFTLTISSLOPDDFATYYCOQYYSSPTF
GGGTKVEIKGGGGSGGGGSGGGGSEVQLVESGGGLVQPGGSLRVSCAASGFTFSS
YAMSWVRQTNKKECPEDYTYNPRCPQQYGWSDCDCMGDRFGGYCRQDGCSNYIH
RSTLEWVSAISGLGGSTYYADSVKGRFTISRDNSKNTLYLQMNSLRAEDTAVYY
CAKDHRVWAAGYHFDYWGQGTLVTVSSGGGGGGGGSGGGGSGGGGS

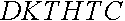

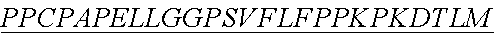

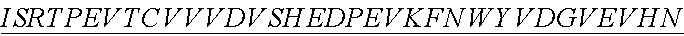

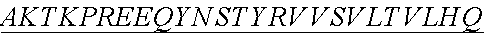

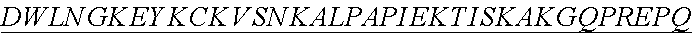

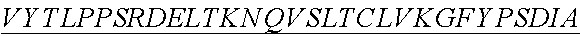

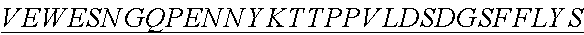

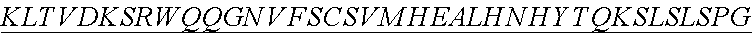

Note: The underlined indicates A DI-15878 variable light region; the bolded font indicates
ADI-15878 variable heavy region; double underline indicates UL-CDR3 from NC-Cow1; the
wavy underline indicates amino acid sequence from human IgG Fc.
SEQ ID NO: 34
Amino acid sequence of ODIN-11 Fc V1 | ODIN-11 fused to human
IgG 1 constant domain
METDTLLLWVLLLWVPGSTGDIQMTOSPSTLSASVGDRVTITCRASQSISSWLAWYQ
QTNKKECPEDYTYNPRCPQQYGWSDCDCMGDRFGGYCRQDGCSNYIHRSTVKLLIS
DASSLESGVPSRFSGSGSGTEFTLTISSLOPDDFATYYCOQYYSSPTFGGGTKVEIKGG
GGSGGGGSGGGGSEVQLVESGGGLVQPGGSLRVSCAASGFTFSSYAMSWVRQAP
GKGLEWVSAISGLGGSTYYADSVKGRFTISRDNSKNTLYLQMNSLRAEDTAVYY
CAKDHRVWAAGYHFDYWGQGTLVTVSSGGGGSGGGGSGGGGSGGGGS

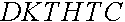

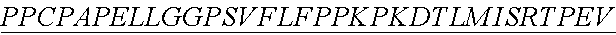

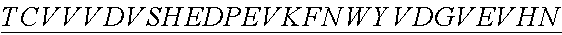

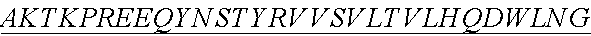

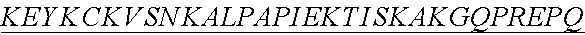

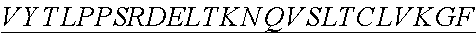

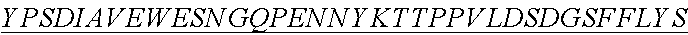

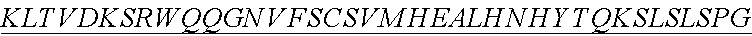

Note: The underlined indicates A DI-15878 variable light region; the bolded font indicates
ADI-15878 variable heavy region; double underline indicates UL-CDR3 from NC-Cow1; the
wavy underline indicates amino acid sequence from human IgG Fc.
SEQ ID NO: 35
Amino acid sequence of VHH -72/Gly | V HH 72.58 with a
glycosylation site in the F R 2 loop
METDTLLLWVLLLWVPGSTGDQVQLQESGGGLVQAGGSLRLSCAASGRTFSEYAM
GWFRQGSSGNSTGSSGEREFVATISWSGGSTYYTDSVKGRFTISRDNAKNTVYLQM
NSLKPDDTAVYY CAAAGLGTVVSEWDYDYDYWGQGTQVTVSSGSSGSGSGSESAW
SHPQFEKGGGSGGGSGGSSAWSHPQFEK
Note: The underlined indicates VHH 72; the bolded font indicates a glycosylation site (NST)
flanked by two GSSG linkers.

1-80. (canceled)

81. An antigen-binding protein construct, comprising:
(a) an antigen-binding protein scaffold with at least one variable heavy (VH), variable light (VL) or VHH domain, said domain having an F2 region;
(b) a heterologous polypeptide inserted into said F2 region of said protein scaffold domain to produce a bifunctional antigen-binding protein construct.

82. The construct of claim 81, further comprising:
a peptide linker of 2 to 50 amino acids coupling said F2 region of said antigen-binding protein scaffold and said heterologous polypeptide.

83. The construct of claim 82, wherein the linker comprises an amino acid sequence of the group of GSSG and GSSGSSG.

84. The construct of claim 81, said F2 region further comprising:
at least one a glycosylation site.

85. The construct of claim 84, wherein the glycosylation site comprises an amino acid sequence of NXT or NXS, wherein X is any amino acid except proline.

86. The construct of claim 81, further comprising a detectable marker.

87. The construct of claim 86, wherein the detectable marker is a marker selected from the group consisting of a peptide tag, a histidine tag, a hemagglutinin tag, a flag tag, a myc tag, a strep tag, and an A56R protein tag.

88. The construct of claim 87, wherein the hemagglutinin tag has an amino acid sequence YPYDVPDYA, the flag tag has an amino acid sequence DYKDDDDK, the myc tag has an amino acid sequence EQKLISEEDL and the strep tag has an amino acid sequence of WSHPQFEK.

89. The construct of claim 81, wherein said heterologous polypeptide is inserted immediately C-terminal to amino acid 43, 44, 45, 46, 47, 48, or 49 of the FR2 region (IMGT numbering).

90. The construct of claim 89, wherein the heterologous polypeptide is inserted immediately C-terminal to amino acid 43 or 44 of the FR2 region (IMGT numbering).

91. The construct of claim 81, wherein the heterologous polypeptide comprises an oligomerization domain.

92. The construct of claim 81, wherein the oligomerization domain is selected from the group consisting of a GCN4 leucine zipper, a phage T4 fibritin foldon domain, a Comp48, and an oligomeric beta sheet.

93. The construct of claim 81, wherein the heterologous polypeptide comprises a polypeptide that extends serum half-life selected from the group of an albumin-binding protein, an anti-albumin antibody or a fragment thereof, albumin, an immunoglobulin, an Fc domain, a fragment of an Fc domain, and an FcRnBP.

94. The construct of claim 81, wherein the heterologous polypeptide comprises a protein selected from the group of a cytokine and a chemokine.

95. The construct of claim 81, wherein the heterologous polypeptide comprises PD-1, PD-L1, CTLA-4, B7, or CD3.

96. The construct of claim 81, wherein the heterologous polypeptide comprises at least one natural or unnatural amino acid.

97. The construct of claim 96, wherein the at least one natural or unnatural amino acid, is conjugated to a polyethylene glycol (PEG), a chemotherapeutic agent, and/or a cytotoxic agent.

98. The construct of claim 96, wherein the at least one unnatural amino acid is selected from the group consisting of an azide, alkynes, an aldehyde, an aminooxy, a functionalized arene, or a trans-cyclooctene (e.g., for bio-orthogonal labeling); fluorosulfate-L-tyrosine (FSY); L-Azidohomoalanine hydrochloride; L-Azidonorleucine hydrochloride; p-acetylphenylalanine (pAcPhe); para-acetylphenylalanine (pAF); para-azidophenylalanine (pAZ); N6-((2-azidoethoxy)carbonyl)-I-lysine; a cysteine and selenocysteine derivative; a leucine derivative; a phenylalanine derivative; a lysine derivative; a tryptophan derivative; and/or
a tyrosine derivative.

99. The construct of claim 81, wherein the heterologous polypeptide comprises a second antigen-binding protein or protein subdomain.

100. The construct of claim 99, wherein the antigen-binding protein comprises an ultralong CDR3 (UL-CDR3), scFV, Fab′, F(ab′)2, ds-scFv, scFab′, diabody, scFV-CH3 (minibody), a VHH domain, a VH domain, a VL domain, or a VNAR domain.
